(19) United States
(12) Patent Application Publication
(10) Pub. No.: US 2016/0194694 A1
(43) Pub. Date: Jul. 7, 2016

(54) MULTIPLEX TRANSCRIPTOME ANALYSIS

(71) Applicant: LIFE TECHNOLOGIES CORPORATION, Carlsbad, CA (US)

(72) Inventors: Kelli BRAMLETT, Austin, TX (US); John LEAMON, Stonington, CT (US); Mark ANDERSEN, Carlsbad, CA (US); Michael THORNTON, San Francisco, CA (US)

(21) Appl. No.: 15/068,466

(22) Filed: Mar. 11, 2016

Related U.S. Application Data
(63) Continuation-in-part of application No. 14/826,385, filed on Aug. 14, 2015.
Application No. 14/826,385 is a continuation-in-part of application No. 13/458,739, filed on Apr. 27, 2012.
Application No. 13/458,739 is a continuation-in-part of application No. 14/795,826, filed on Jul. 9, 2015.
(60) Provisional application No. 62/037,575, filed on Aug. 14, 2014.
(60) Provisional application No. 62/046,845, filed on Sep. 5, 2014.
(60) Provisional application No. 61/479,952, filed on Apr. 28, 2011.
(60) Provisional application No. 61/531,583, filed on Sep. 6, 2011.
(60) Provisional application No. 61/531,574, filed on Sep. 6, 2011.
(60) Provisional application No. 61/538,079, filed on Sep. 22, 2011.
(60) Provisional application No. 61/564,763, filed on Nov. 29, 2011.
(60) Provisional application No. 61/578,192, filed on Dec. 20, 2011.
(60) Provisional application No. 61/594,160, filed on Feb. 2, 2012.
(60) Provisional application No. 61/598,881, filed on Feb. 14, 2012.
(60) Provisional application No. 61/598,892, filed on Feb. 14, 2012.

(51) Int. Cl. C12Q 1/68

(52) U.S. Cl. CPC C12Q 1/6855; C12Q 1/6874; C12Q 1/686

(57) ABSTRACT

In some embodiments, the disclosure relates generally to methods, compositions, systems, apparatuses and kits comprising a multiplex nucleic acid amplification reaction that employs a plurality (e.g., hundreds, thousands, tens-of-thousands or hundreds-of-thousands) of different target-specific primer pairs that enable substantially simultaneous amplification of a plurality of different target sequences-of-interest in a single reaction mixture. In some embodiments, the multiplex nucleic acid amplification reaction generates a plurality of amplicons having sequences derived from a sample containing RNA or DNA, including whole transcriptome or genomic samples. In some embodiments, the sequences and abundances of at least some of the plurality of amplicons are characterized, optionally simultaneously or through a single assay, by suitable detection methods, including sequencing or other procedures known in the art.

This application claims the benefit of priority under 35 U.S.C. §119 to U.S. Provisional Application Nos. 62/037,575, filed Aug. 14, 2014, and 62/046,845, filed Sep. 5, 2014; and this application is a continuation-in-part of U.S. Non-provisional application Ser. No. 13/458,739, filed Apr. 27, 2012, which claims priority to U.S. Provisional Application Nos. 61/479,952, filed Apr. 28, 2011, and 61/531,583, filed Sep. 6, 2011, and 61/531,574, filed Sep. 6, 2011, and 61/538,079, filed Sep. 22, 2011, and 61/564,763, filed Nov. 29, 2011, and 61/578,192, filed Dec. 20, 2011, and 61/594,160 filed Feb. 2, 2012, and 61/598,881 filed Feb. 14, 2012, and 61/598,892 filed Feb. 14, 2012; and this application is a continuation-in-part of U.S. Non-provisional application Ser. No. 14/795,826, filed Jul. 9, 2015; the disclosures of all of the which aforementioned applications are incorporated by reference in their entireties.

All literature and similar materials cited in this application, including but not limited to, patents, patent applications, articles, books, treatises, and internet web pages are expressly incorporated by reference in their entirety for any purpose.

In some embodiments, the disclosure relates generally to methods, compositions, systems, apparatuses and kits comprising a multiplex nucleic acid amplification reaction that employs a plurality (e.g., hundreds, thousands, tens-of-thousands or hundreds-of-thousands) of different target-specific primer pairs that enable substantially simultaneous amplification of a plurality of different target sequences-of-interest in a single reaction mixture. In some embodiments, the multiplex nucleic acid amplification reaction generates a plurality of amplicons having sequences derived from a sample containing RNA or DNA, including whole transcriptome or genomic samples. In some embodiments, the sequences and abundances of at least some of the plurality of amplicons are characterized, optionally simultaneously or through a single assay, by suitable detection methods, including sequencing or other procedures known in the art.

### BRIEF DESCRIPTION OF THE DRAWINGS

FIG. 1 shows a tally of the number of different amplicon sequences generated by multiplex amplification of transcripts contained in a Universal Human Reference or Human Brain Reference sample.

### SUMMARY

In some embodiments, the disclosure relates generally to compositions, methods, systems, apparatuses and kits, comprising a plurality of target polynucleotides and a plurality of target-specific primers.

Optionally, the target-specific primers are complementary or identical to at least a portion of one or more target polynucleotides of the plurality of target polynucleotides.

Optionally, at least one of the plurality of target-specific primers is a tailed primer having a portion that hybridizes to a target polynucleotide and a portion that does not hybridize to the target polynucleotide. Optionally, at least one of the plurality of target-specific primers is not a tailed primer.

Optionally, at least one of the primers in the plurality of target-specific primers contains at least one cleavable group.

Optionally, each of the plurality of target-specific primers contains at least one cleavable group.

In some embodiments, the disclosure relates generally to compositions, methods, systems, apparatuses and kits further comprising a cleaving agent capable of cleaving the at least one cleavable group of the plurality of target specific primers.

In some embodiments, the disclosure relates generally to compositions, methods, systems, apparatuses and kits further comprising at least one polymerase.

In some embodiments, the disclosure relates generally to compositions, methods, systems, apparatuses and kits further comprising a plurality of nucleotides.

In some embodiments, the disclosure relates generally to compositions (and related methods of making and/or using, systems, apparatuses and kits) comprising (i) a plurality of target-specific primers each containing at least one cleavable group, (ii) a polymerase, (iii) a cleaving agent capable of cleaving the at least one cleavable group of the plurality of target-specific primers, and (iv) a plurality of target polynucleotides, wherein the target-specific primers are complementary or identical to at least a portion of one or more of the target polynucleotides of the plurality. Optionally, the compositions, systems, apparatuses and kits further include a plurality of nucleotides.

In some embodiments, the disclosure relates generally to compositions, methods, systems, apparatuses and kits, comprising a single reaction mixture which contains (i) a plurality of target-specific primers, (ii) a polymerase, and (iii) a plurality of target polynucleotides, wherein the target-specific primers are complementary or identical to at least a portion of one or more of the target polynucleotides of the plurality. Optionally, each target-specific primer in the single reaction mixture contains at least one cleavable group. Optionally, the single reaction mixture further comprises a cleaving agent capable of cleaving the at least one cleavable group of the plurality of target-specific primers. Optionally, the single reaction mixture further comprises a plurality of nucleotides. Optionally, the single reaction mixture contains at least 1000, 2500, 5000, 7500, 10,000, 12,000, 15,000, 17,500, 20,000, 25,000, 50,000, 100,000, 200,000 or 500,000 different target-specific primers.

Optionally, the plurality of target polynucleotides comprises RNA, DNA or cDNA.

Optionally, the plurality of target polynucleotides includes genomic DNA.

Optionally, the plurality of target polynucleotides includes total RNA, polyA RNA or non-polyA RNA.

Optionally, the plurality of target polynucleotides comprises single-stranded or double-stranded nucleic acids.

Optionally, at least a portion of each of the target-specific primers can hybridize to at least a portion of one or more target polynucleotides of the plurality of target polynucleotides.

In some embodiments, the plurality of target-specific primers includes at least 1000, 2500, 5000, 7500, 10,000, 12,000, 15,000, 17,500, 20,000, 25,000, 50,000, 100,000, 200,000 or 500,000 different target-specific primers.

Optionally, the plurality of nucleotides includes a detectable label.

In some embodiments, the disclosure relates generally to compositions (and related methods of making and/or using, systems, apparatuses and kits) comprising a single reaction mixture which contains: (i) a plurality of target-specific primer pairs each containing at least one cleavable group, (ii) a plurality of target cDNA polynucleotides, wherein the target-specific primer pairs are complementary or identical to at least a portion of one or more of the target cDNA polynucleotides of the plurality, (iii) a polymerase, and (iv) a plurality of nucleotides. In some embodiments, the single reaction mixture further comprises: (v) a cleaving agent capable of cleaving the at least one cleavable group of the plurality of target-specific primer pairs. Optionally, the plurality of target-specific primer pairs can hybridize to about 100-100,000 different target cDNA sequences. Optionally, within the plurality of target-specific primer pairs, each pair of target-specific primer pairs is configured to hybridize to one target polynucleotide.

In some embodiments, the disclosure relates generally to compositions (and related methods of making and/or using, systems, apparatuses and kits) comprising any two or more, in any combination of: a plurality of target-specific primers each containing at least one cleavable group; a polymerase; a cleaving agent capable of cleaving the at least one cleavable group of the plurality of target-specific primers; a plurality of target polynucleotides wherein the target-specific primers are complementary or identical to at least a portion of one or more of the target polynucleotides of the plurality; and/or a plurality of nucleotides. Optionally, the plurality of target-specific primers includes at least 1000, 2500, 5000, 7500, 10,000, 12,000, 15,000, 17,500, 20,000, 25,000, 50,000, 100,000, 200,000 or 500,000 different target-specific primers. Optionally, at least a portion of each of the target-specific primers can hybridize to at least a portion of the one or more target polynucleotides of the plurality of target polynucleotides. Optionally, the plurality of target polynucleotides comprises single-stranded or double-stranded nucleic acids. Optionally, the plurality of target polynucleotides includes RNA, DNA, cDNA, a mixture of RNA and DNA, or genomic DNA. Optionally, the plurality of target polynucleotides includes naturally-occurring, recombinant or synthetically prepared forms. Optionally, the plurality of target polynucleotides includes amplification products (e.g., amplicons) or fragmentation products (e.g., fragments). Optionally, the plurality of target polynucleotides is derived from RNA, DNA, cDNA, a mixture of RNA and DNA, or genomic DNA. Optionally, the plurality of target polynucleotides includes total RNA, polyA RNA or non-polyA RNA. Optionally, the plurality of nucleotides includes a detectable label, or the plurality of nucleotides are unlabeled, or a mixture of labeled and unlabeled nucleotides.

In some embodiments, the disclosure relates generally to compositions, methods, systems, apparatuses and kits further comprising a ligase.

Optionally, the ligase comprises a DNA or RNA ligase.

In some embodiments, the disclosure relates generally to compositions, methods, systems, apparatuses and kits further comprising one or more adaptors.

Optionally, the one or more adaptors are not complementary or identical to the 5′ end of the plurality of target-specific primers.

Optionally, the one or more adapters do not include a nucleic acid sequence that is complementary or identical to the terminal 10 nucleotides at the 5′ end of the plurality of target-specific primers.

Optionally, the one or more adapters comprise a universal priming sequence, a tag, or a unique identifier sequence (e.g., barcode sequence).

Optionally, the universal priming sequence comprises an amplification priming sequence or a sequencing priming sequence.

Optionally, at least one of the one or more adaptors is phosphorylated at the 5′ end.

Optionally, a plurality of the one or more adaptors is single-stranded or double-stranded.

In some embodiments, the plurality of target-specific primers includes at least 1000, 2500, 5000, 7500, 10,000, 12,000, 15,000, 17,500, 20,000, 25,000, 50,000, 100,000, 200,000 or 500,000 different target-specific primer pairs.

In some embodiments, the plurality of target polynucleotides is derived from RNA.

Optionally, the RNA is derived from a single cell or from a population of cells.

Optionally, the RNA is derived from a cancer cell, oocyte, embryo, stem cell, or cell exposed to a companion diagnostic compound.

In some embodiments, the plurality of target polynucleotides includes a plurality of cDNAs that are transcribed from a transcriptome.

In some embodiments, the transcriptome comprises a population of RNA that is produced (e.g., transcribed) in one or more cells.

Optionally, the transcriptome comprises a population of RNA that is produced by transcription of one or more genes in a single cell or in a plurality of cells.

Optionally, the transcriptome comprises a population of RNA having a mixture of different sequences.

Optionally, the transcriptome comprises different RNA sequences present in different amounts (e.g., abundance).

Optionally, the population of RNA contained in a transcriptome represents one or more genes expressed in a single cell or in a plurality of cells.

In some embodiments, the plurality of target polynucleotides contain sequences that are derived from one or more RNA sequences isolated from a single cell or from a plurality of cells.

Optionally, the plurality of target polynucleotides contain sequences that are derived from one or more expressed genes a single cell or from a plurality of cells.

Optionally, one or more target polynucleotides of the plurality contain sequences that represent one or more genes that are expressed in a single cell or in a plurality of cells.

Optionally, the plurality of target polynucleotides includes a plurality of cDNAs that collectively represent RNA expression in a single cell or in a plurality of cells.

Optionally, the plurality of target polynucleotides includes a plurality of cDNAs that represent mRNA expression in the transcriptome.

Optionally, the plurality of target polynucleotides includes different sequences that are derived from a transcriptome, where the transcriptome represents one or more genes expressed in a single cell or in a plurality of cells.

In some embodiments, the plurality of target-specific primers are complementary or identical to at least some portion of an RNA transcribed in vitro or in vivo from one or more of the genes selected from the group consisting of ABL1; AKT1; ALK; APC; ATM; BRAF; CDH1; CDKN2A; CSF1R; CTNNB1; EGFR; ERBB2; ERBB4; FBXW7; FGFR1; FGFR2; FGFR3; FLT3; GNAS; HNF1A; HRAS; IDH1; JAK2; JAK3; KDR; KIT; KRAS; MET; MLH1; MPL; NOTCH1; NPM1; NRAS; PDGFRA; PIK3CA; PTEN; PTPN11; RB1; RET; SMAD4; SMARCB1; SMO; SRC; STK11; TP53; and VHL.

In some embodiments, the plurality of target-specific primers includes a plurality of target-specific primer pairs, at least one primer pair including a forward primer and a reverse primer and being configured to amplify at least some portion of a single target polynucleotide of the plurality of target polynucleotides. Optionally, the plurality of target-specific primer pairs includes at least two different primer pairs configured to amplify a polynucleotide sequence from different respective target polynucleotides. Optionally, each primer pair in the plurality of target-specific primer pairs is configured to amplify a polynucleotide sequence from a different target polynucleotide than any other primer pair.

In some embodiments, the plurality of target-specific primers includes two or more pairs of target-specific primers configured to amplify any given target polynucleotide. Optionally, any given target polynucleotide can hybridize with two or more different pairs of target-specific primers, and be subjected to a primer extension reaction to yield two or more different amplification products. Optionally, each pair in the two or more pairs of target-specific primers comprises a forward and a reverse primer.

In some embodiments, the plurality of target polynucleotides includes a first target polynucleotide, and the plurality of target-specific primers includes only a single pair of target-specific primers configured to amplify the first target polynucleotide. Optionally, the single pair of target-specific primers comprises two different target-specific primers that are either complementary or identical to at least some portion of the first target polynucleotide.

In some embodiments, any target polynucleotide of the plurality of target polynucleotides can hybridize to only a single pair of target-specific primers.

In some embodiments, any target polynucleotide of the plurality of target polynucleotides contains a sequence that is complementary or identical to only a single pair of target-specific primers.

In some embodiments, each of the different pairs of target-specific primers, in the plurality of target-specific primers, is complementary or identical to a different target polynucleotide.

In some embodiments, the disclosure relates generally to compositions, methods, systems, apparatuses and kits further comprising a plurality of amplicons.

In some embodiments, the amplicons are formed by hybridizing one or more of the plurality of target-specific primers to one or more of the plurality of target polynucleotides, and extending at least one of the one or more hybridized target-specific primers in a template dependent manner.

In some embodiments, the amplicons include a polynucleotide formed by amplification of at least a portion of a target polynucleotide using only a single pair of the target-specific primers of the plurality of target-specific primers.

In some embodiments, the disclosure relates generally to compositions, methods, systems, apparatuses and kits, comprising (i) a plurality of target polynucleotides, (ii) 1000 target-specific primers, each primer including a cleavable group, (iii) at least one polymerase, (iv) a cleaving reagent capable of cleaving the cleavable group of the target-specific primers, and (v) a plurality of nucleotides.

In some embodiments, the plurality of target polynucleotides is derived from a cell population.

In some embodiments, the plurality of target polynucleotides is formed by reverse transcription of RNA extracted from a cell population.

In some embodiments, the plurality of target polynucleotides includes a plurality of cDNAs formed by reverse transcription of total mRNA extracted from a cell population.

Optionally, the total mRNA includes at least one RNA transcript having a mutant sequence and the plurality of target polynucleotides includes at least one cDNA derived from the mutant sequence.

Optionally, the RNA transcript having the mutant sequence is associated with a disease or cancer.

Optionally, the RNA transcript having the mutant sequence includes an abnormal splice junction sequence.

Optionally, the abnormal splice junction sequence includes an abnormal exon-exon splice junction sequence, an abnormal exon-intron splice junction sequence, an abnormal intron splice junction sequence, an abnormal intra-exon splice junction sequence, or an abnormal intra-intron splice junction sequence

Optionally, the RNA transcript having the mutant sequence includes an abnormal splice transcript sequence.

Optionally, the RNA transcript having the abnormal splice junction sequence is associated with a disease or cancer.

In some embodiments, only a single pair of primers of the 1000 target-specific primers hybridizes to any given target polynucleotide of the composition.

In some embodiments, the plurality of target polynucleotides is obtained by reverse transcribing RNA.

Optionally, the plurality of target polynucleotides is obtained by reverse transcribing a plurality of RNA transcripts from a sample.

In some embodiments, the plurality of target polynucleotides includes DNA.

Optionally, the plurality of target polynucleotides includes genomic DNA.

Optionally, the plurality of nucleotides includes a detectable label.

In yet another embodiment, the composition (as well as related methods, systems, apparatuses and kits) includes a plurality of target-specific primers each containing at least one cleavable group, a cleaving reagent capable of cleaving the at least one cleavable group of a plurality of the target-specific primers, a polymerase, and a plurality target polynucleotides where the target-specific primers are complementary to at least a portion of one or more target polynucleotides of the plurality of target polynucleotides. In another embodiment, the composition (as well as related methods, systems, apparatuses and kits) includes a plurality of target-specific primers each containing at least one cleavable group, a cleaving reagent capable of cleaving the at least one cleavable group of a plurality of the target-specific primers, a polymerase, and a plurality target polynucleotides where the target-specific primers are identical to at least a portion of one or more target polynucleotides of the plurality of target polynucleotides. In some embodiments, the composition further includes a plurality of nucleotides. In some embodiments, the composition includes 1000, 2000, 5000, 10000, 20000, 25000, 25000, 50000, 100000, 200000 or 500000 different target-specific primers. In yet another embodiment, the composition includes at least 1000, 2500, 5000, 7500, 10000, 12000, 15000, 17500, 20000, 25000, 50000, 100000, 200000 or 500000 target-specific primer pairs. In some embodiments, the composition includes at least some of the plurality of target-specific primers that are complementary or identical to at least some portion of an RNA transcribed in vitro or in vivo from one or more of the genes selected from the group consisting of ABL1; AKT1; ALK; APC; ATM; BRAF; CDH1; CDKN2A; CSF1R; CTNNB1; EGFR; ERBB2; ERBB4; FBXW7; FGFR1; FGFR2; FGFR3; FLT3; GNAS; HNF1A; HRAS; IDH1; JAK2; JAK3; KDR; KIT; KRAS; MET; MLH1; MPL; NOTCH1; NPM1; NRAS; PDGFRA; PIK3CA; PTEN; PTPN11; RB1; RET; SMAD4; SMARCB1; SMO; SRC; STK11; TP53; and VHL. In some embodiments, at least some of the target-specific primers are complementary to at least some portion of an RNA transcribed in vitro or in vivo from one or more active genes of the RNA transcriptome. In some embodiments, the composition includes a plurality of target-specific primers where only a single pair of target-specific primers is complementary to any target polynucleotide of the plurality of target polynucleotides. In another embodiment, the composition includes a plurality of amplicons formed by hybridizing one or more of the plurality of target-specific primers to one or more of the plurality of target polynucleotides and extending at least one of the one or more hybridized target-specific primers in a template dependent manner. In yet another embodiment, the plurality of amplicons formed by hybridizing one or more of the plurality of target-specific primers to one or more of the plurality of target polynucleotides and extending at least one of the one or more hybridized target-specific primers in a template dependent manner is formed via amplification of at least a portion of a target polynucleotide using only a single pair of target-specific primers from the plurality of target-specific primers.

In yet another embodiment, the composition (as well as related methods, systems, apparatuses and kits) includes 1000 target-specific primers each including a cleavable group, a cleaving reagent capable of cleaving the cleavable group, a polymerase, a plurality target polynucleotides, and a plurality of nucleotides. In some embodiments, only a single pair of target-specific primers of the 1000 target-specific primers hybridizes to any given target polynucleotide of the composition. In some embodiments, the plurality of nucleotides includes a detectable label or nucleotide analog. In some embodiments, the plurality of target polynucleotides can be obtained by reverse transcribing a plurality of RNA transcripts from a sample. In some embodiments, the plurality of target polynucleotides can include genomic DNA or cDNA. In some embodiments, the cDNA represents mRNA expression in a RNA transcriptome. In some embodiments, the plurality of target polynucleotides includes RNA. In some embodiments, the plurality of target polynucleotides can include RNA derived from a single cell or from a population of cells. In some embodiments, the amount of DNA or cDNA required can be 200 pg to 1 microgram. In some embodiments, the amount of DNA or cDNA required for amplification of one or more of the target polynucleotides can be 200 pg to 100 ng, 500 pg to 50 ng, 1 ng to 25 ng, or 1 ng to 10 ng. In one embodiment, the amount of DNA or cDNA required for amplification of one or more of the plurality of target polynucleotides by one or more methods disclosed herein is 1 ng to 25 ng.

In some embodiments, the number of target polynucleotides amplified by one or more of the methods using the compositions (as well as related kits, apparatuses and systems) disclosed herein can be hundreds, thousands, or hundreds of thousands of target polynucleotides in a single reaction mixture. In some embodiments, the number of different target polynucleotides amplified in a single multiplex amplification reaction can be at least 1000, 2000, 5000, 10000, 20000, 25000, 50000, 100000, 12500, 15000, 200000, 300000, 400000, or 500000, or greater.

In some embodiments, the disclosure relates generally to methods (as well as related compositions, systems, apparatuses and kits) for synthesizing a plurality of polynucleotides in a sample, comprising synthesizing a plurality of amplicons, wherein the synthesizing includes forming a reaction mixture by contacting a plurality of target polynucleotides with a plurality of target-specific primer pairs. Optionally, at least one of the plurality of target-specific primers is a tailed primer. Optionally, at least one of the plurality of target-specific primers is a non-tailed primer. The methods can further include extending at least one primer of a target-specific primer pair to form one or more synthesized polynucleotides. The methods can include extending both primers of a target specific primer pair, either simultaneously or sequentially, optionally using isothermal or non-isothermal conditions. The extending can include extending in a template-dependent or template-directed manner. In some embodiments, the extending includes forming one or a plurality of amplicons. At least one amplicon is optionally formed via amplification of a single target polynucleotide using at least one pair of target-specific primers. In some embodiments, the methods include forming one or a plurality of amplicons, each amplicon being formed by amplifying a single target polynucleotide using only a single target-specific primer pair. Optionally, the methods further include detecting at least some of the amplicons, for example using optical or non-optical detection. In some embodiments, the methods further include obtaining sequence information from one or a plurality of the amplicons. In some embodiments, when a single primer pair is used to generate a single sequence for each target polynucleotide, a sequence read assembly is not performed.

In some embodiments the plurality of target polynucleotides contains a mixture of different target sequences.

In some embodiments the plurality of target polynucleotides contains at least a first and a second target polynucleotide.

In some embodiments the sequence of the different target polynucleotides in the plurality can be directly extracted or otherwise derived from a sample containing RNA or DNA, or a mixture of both. Optionally, the sample comprises a whole transcriptome, or a portion of a whole transcriptome. Optionally, the sample comprises a genome or a portion of a genome. Optionally, the sample contains RNA, DNA, cDNA, or recombinant DNA or RNA derived from one or more cells. Optionally, the sample contains total RNA derived from one or more cells. In some embodiments, the different target polynucleotides include a plurality of RNA molecules forming a transcriptome, or a plurality of cDNA molecules formed via reverse transcription of a transcriptome, or a plurality of DNA molecules formed via amplification and/or fragmentation of a transcriptome or a genome.

In some embodiments, the methods further include hybridizing different target-specific primer pairs from the plurality of target-specific primer pairs to different target polynucleotides. Optionally, the disclosed methods include hybridizing each pair of two or more different target-specific primer pairs to different target polynucleotides.

In some embodiments, the methods further include hybridizing at least a portion of each primer of a target-specific primer pair to a portion of a corresponding target polynucleotide, or its complement.

In some embodiments, the plurality of target-specific primers includes a plurality of target-specific primer pairs, at least one primer pair including a forward primer and a reverse primer and being configured to amplify at least some portion of a single target polynucleotide of the plurality of target polynucleotides. Optionally, the plurality of target-specific primer pairs includes at least two different primer pairs, each primer pair configured to amplify a polynucleotide sequence from a target polynucleotide, where no two primer pairs are configured to amplify a polynucleotide sequence from the same target polynucleotide. Optionally, each primer pair from the plurality of different primer pairs is configured to amplify polynucleotide sequences from different target polynucleotides. Optionally, each primer pair in the plurality of target-specific primer pairs is configured to amplify a polynucleotide sequence from a different target polynucleotide than any other primer pair.

In some embodiments, the plurality of target polynucleotides includes a first target polynucleotide, and the plurality of target-specific primers includes only a single pair of target-specific primers configured to amplify the first target polynucleotide. Optionally, the single pair of target-specific primers comprises a forward target-specific primer and a reverse target-specific primer that are either substantially complementary or substantially identical to at least some portion of the first target polynucleotide. Optionally, the first target-specific primer and the reverse target-specific primer hybridize to the first target polynucleotide or its complement under high-stringency hybridization conditions.

In some embodiments, the methods further include hybridizing at least a portion of both target-specific primers of a first target-specific primer pair, each independently and separately, to a portion of a first target polynucleotide or its complement. Optionally, the disclosed methods include hybridizing at least a portion of both target-specific primers of a first target-specific primer pair to a portion of a first target polynucleotide, or its complement. Optionally, the hybridizing includes high stringency hybridization conditions.

In some embodiments, the methods further include hybridizing at least a portion of both target-specific primers of a second target-specific primer pair, each independently and separately, to a portion of a second target polynucleotide, or its complement. Optionally, the disclosed methods include hybridizing at least a portion of both target-specific primers of a second target-specific primer pair to a portion of a second target polynucleotide, or its complement.

In some embodiments, the methods further include hybridizing different pairs of target-specific primers from the plurality of target-specific primer pairs, each independently and separately, to different target polynucleotides, or their complements, to form a plurality of different primer/polynucleotide complexes. In some embodiments, the disclosed methods optionally include forming a plurality of different primer/polynucleotide complexes. In some embodiments, the forming includes hybridizing different pairs of target-specific primers from the plurality of target-specific primer pairs to different target polynucleotides or their complements. In some embodiments, the forming includes extending one or more target-specific primers within different primer/polynucleotide complexes, optionally in a template-dependent manner, for example by using a target polynucleotide of the complex as a template.

In some embodiments, the methods further include hybridizing a plurality of target-specific primer pairs having extendible 3′ ends in a primer extension reaction. In some embodiments, the disclosed methods including extending at least some of the target-specific primer pairs, optionally in a template-dependent manner, for example by using a target polynucleotide of the complex as a template.

In some embodiments, the methods further include hybridizing a plurality of target polynucleotides with the plurality of target-specific primer pairs in a single reaction mixture.

In some embodiments, the methods further include contacting a plurality of target polynucleotides with the plurality of target-specific primer pairs in a single reaction mixture.

In some embodiments, the methods further include contacting, in a single reaction mixture, a first target polynucleotide with a first target-specific primer pair, and contacting a second target polynucleotide with a second target-specific primer pair.

In some embodiments, at least one of the target-specific primer pairs has minimal cross-hybridization with any other pair of primers in the single reaction mixture.

Optionally, the single reaction mixture contains 1000, 2500, 5000, 7500, 10,000, 12,000, 15,000, 17,500, 20,000, 25,000, 50,000, 100,000, 200,000, 500,000, or more than 500,000 different target-specific primer pairs. Optionally, the single reaction mixture contains at least 1000, 2500, 5000, 7500, 10,000, 12,000, 15,000, 17,500, 20,000, 25,000, 50,000, 100,000, 200,000 or 500,000, or more than 500000 different target-specific primer pairs.

In some embodiments, the contacting step is conducted under nucleic acid hybridization conditions such that different target-specific primer pairs hybridize to their cognate target sequences. In some embodiments, the contacting includes contacting the target-specific primer pairs with target polynucleotides and hybridizing at least one member of each pair with a target polynucleotide or its complement.

In some embodiments, the contacting is performed under standard nucleic acid hybridization conditions. In some embodiments, the contacting is performed using stringent hybridization conditions.

In some embodiments, only a single pair of target-specific primers hybridize to any given target polynucleotide. The disclosed methods optionally include hybridizing only a single pair of target-specific primer pairs to a given target polynucleotide.

In some embodiments, the method further includes extending the plurality of primer/polynucleotide complexes in a primer extension reaction.

In some embodiments, the method further includes extending the target-specific primer pairs in primer extension reaction.

In some embodiments, the method further includes extending the target-specific primer pairs in a template-dependent manner.

In some embodiments, the method further includes extending the target-specific primer pairs to form a plurality of amplicons.

In some embodiments, the method further includes conducting a primer extension reaction to form a plurality of amplicons containing sequences derived from the plurality of target polynucleotides. In some embodiments, the methods further include forming a plurality of amplicons containing sequences derived from the plurality of target polynucleotides by extending the target-specific primer pairs in a primer extension reaction.

In some embodiments, the methods further include extending the first target-specific primer pair in a template-dependent manner to form a first amplicon, and extending the second target-specific primer pair in a template-dependent manner to form a second amplicon.

In some embodiments, the each amplicon contains sequences derived from a target polynucleotide.

In some embodiments, at least two of the plurality of amplicons have sequences that are less than 50% complementary to each other

In some embodiments, the first amplicon contains sequences derived from the first target polynucleotide.

In some embodiments, the second amplicon contains sequences derived from the second target polynucleotide.

In some embodiments, the methods further include detecting the plurality of amplicons.

In some embodiments, the detecting includes sequencing at least a portion of the amplicons.

In some embodiments, since a single primer pair is used to generate a single sequence for each target polynucleotide, a sequence read assembly is not performed.

In some embodiments, the disclosed methods include quantifying or otherwise estimating the number of amplicons containing a sequence derived from a given target gene of interest (e.g., a first target gene).

Optionally, the quantifying includes counting or otherwise estimating the number of amplicons containing a target polynucleotide sequence of interest to obtain a number. For example, the quantifying can include counting the number of amplicons containing a first polynucleotide sequence to obtain a first number. In some embodiments, the quantifying includes identifying a first number of amplicons as containing a first polynucleotide sequence. The first number can be the number of amplicons identified as containing the first polynucleotide sequence.

In some embodiments, the disclosed methods further include using the first number to estimate the level of representation of the first target gene, or the first nucleic acid sequence, within the plurality of target polynucleotides.

Optionally, the quantifying includes counting the number of amplicons containing a sequence that maps to the first target gene.

In some embodiments, the first polynucleotide sequence is included in the first target gene.

In some embodiments, the disclosed methods include estimating the number of polynucleotides containing of the first nucleic acid sequence within the plurality of target polynucleotides using the first number.

In some embodiments, the quantifying can include counting the number of amplicons containing a second polynucleotide sequence to obtain a second number. In some embodiments, the quantifying includes identifying a second number of amplicons as containing a second polynucleotide sequence. The second number can be the number of amplicons identified as containing the second polynucleotide sequence.

In some embodiments, the disclosed methods further include using the second number to estimate the level of representation of the second target gene, or the second nucleic acid sequence, within the plurality of target polynucleotides.

Optionally, the quantifying includes counting the number of amplicons containing a sequence that maps to the second target gene.

In some embodiments, the second polynucleotide sequence is included in the second target gene.

In some embodiments, the disclosed methods include estimating the number of polynucleotides containing of the second nucleic acid sequence within the plurality of target polynucleotides using the second number.

In some embodiments, the disclosed methods include determining the amount of the first target polynucleotide and/or the amount of the second target polynucleotide present in the reaction mixture. Optionally, the determining can include using the first number and the second number. In some embodiments, the methods can include inferring or otherwise determining the amount of first polynucleotide sequence and/or the amount of the second polynucleotide sequence in a biological sample.

In some embodiments, the sample includes RNA, DNA or cDNA derived from one or more cells.

In some embodiments, the reaction mixture can include at least some portion of the sample. In some embodiments, the plurality of target polynucleotides in the reaction mixture is extracted directly from the sample, or is derived via manipulation of polynucleotides extracted from the sample.

In some embodiments, the plurality of target-specific primer pairs includes 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target-specific primer pairs.

In some embodiments, the single reaction mixture contains about 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target-specific primer pairs.

In some embodiments, the primer extension reaction can form a plurality of amplicons containing sequences derived from 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target polynucleotides.

In some embodiments, the plurality of polynucleotides can be detected by quantifying the number of amplicons containing sequence derived from each of the 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target polynucleotides.

In some embodiments, the sample includes nucleic acids (e.g., RNA, DNA or cDNA) derived from one or more cells and the method further includes quantifying the amounts for each of 2-100, or about 100-500, or about 500-1,000, or about 1,000-5,000, or about 5,000-10,000, or about 10,000-15,000, or about 15,000-20,000, or about 20,000-25,000, or about 25,000-50,000, or about 50,000-100,000, or about 100,000-200,000, or about 200,000-500,000, or more different nucleic acids present in the sample.

In some embodiments, the sample includes cDNA derived from RNA (e.g., total cellular RNA) and the method further includes quantifying the amounts for each of the 2-100, or about 100-500, or about 500-1,000, or about 1,000-5,000, or about 5,000-10,000, or about 10,000-15,000, or about 15,000-20,000, or about 20,000-25,000, or about 25,000-50,000, or about 50,000-100,000, or about 100,000-200,000, or about 200,000-500,000, or more different transcripts present in the sample.

In some embodiments, methods for detecting a plurality of polynucleotides in a sample further comprise hybridizing the plurality of amplicons with a labeled or un-labeled nucleic acid probe, with a microarray or with a nucleic acid having a reference sequence.

In some embodiments, methods for detecting a plurality of polynucleotides in a sample further comprise re-amplifying the plurality of amplicons.

In some embodiments, the method further comprises calculating a ratio of the first number and the second number. Optionally, the first number represents the number of amplicons derived from the first target gene, and the second number of amplicons represents the number of amplicons derived from the second target gene. Optionally, the first number represents the number of amplicons containing a first polynucleotide sequence of interest, and the second number represents the number of amplicons containing a second polynucleotide sequence of interest.

In some embodiments, the disclosed methods further include sequencing at least some or substantially all of the plurality of adaptor-ligated amplified target sequences.

Optionally, the sequencing comprises a massively parallel sequencing procedure or a gel electrophoresis procedure.

In some embodiments, methods for detecting a plurality of polynucleotides in a sample further comprise comparing the number of plurality of adaptor-ligated amplified target sequences containing the sequence derived from the first polynucleotide with the number of plurality of adaptor-ligated amplified target sequences containing the sequence derived from the second polynucleotide.

In some embodiments, methods for detecting a plurality of polynucleotides in a sample further comprise determining the abundance in the reaction mixture of the plurality of adaptor-ligated amplified target sequences containing the sequence derived from the first polynucleotide relative to the number of plurality of adaptor-ligated amplified target sequences containing the sequence derived from the second polynucleotide within the same reaction mixture or within a different reaction mixture.

In some embodiments, methods for detecting a plurality of target polynucleotides in a sample further comprise calculating a ratio of the number of plurality of adaptor-ligated amplified target sequences containing the sequence derived from the first target polynucleotide and the number of plurality of adaptor-ligated amplified target sequences containing the sequence derived from the second target polynucleotide.

In some embodiments, the extending step (e.g., primer extending) includes forming a plurality of amplicons, wherein each amplicon contains a primer-derived sequence on at least one end, and each amplicon contains a sequence derived from a target polynucleotide

In some embodiments, the each amplicon contains sequences derived from at least one target specific primer.

In some embodiments, the first and the second amplicons contain sequences derived from at least one target specific primer.

In some embodiments, at least one of the primers from plurality of target-specific primer pairs includes a cleavable group.

In some embodiments, the plurality of amplicons includes a primer-derived sequence on at least one end, and the primer-derived sequence includes at least one cleavable group.

Optionally, the at least one cleavable group comprises uracil, uridine, inosine, or 7,8-dihydro-8-oxoguanine (8-oxoG) nucleobases.

Optionally, the at least one cleavable group is cleavable with an enzyme, chemical compound, heat or light.

Optionally, the at least one cleavable group is cleavable with uracil DNA glycosylase (UDG, also referred to as UNG), formamidopyrimidine DNA glycosylase (Fpg), or a FuPa reagent.

In some embodiments, the methods for detecting a plurality of polynucleotides in a sample further comprise cleaving the cleavable groups on the primer-derived sequences on the ends of the plurality of amplicons thereby producing a plurality of cleaved amplified target sequences.

In some embodiments, the methods for detecting a plurality of polynucleotides in a sample further comprise producing a plurality of adaptor-ligated amplified target sequences by ligating one or more adaptors to one or both ends of the plurality of cleaved amplified target sequences.

In some embodiments, at least one of the one or more adaptors includes a unique identifier sequence.

In some embodiments, at least one of the one or more adaptors includes a sequencing primer binding site, an amplification primer binding site or a universal sequence.

In some embodiments, methods for detecting a plurality of polynucleotides in a sample further comprise hybridizing the plurality of adaptor-ligated amplified target sequences with a labeled or un-labeled nucleic acid probe, with a microarray or with a nucleic acid having a reference sequence.

In some embodiments, methods for detecting a plurality of polynucleotides in a sample further comprise re-amplifying the plurality of adaptor-ligated amplified target sequences.

In some embodiments, the method further comprises calculating a ratio of the number of a first adaptor-ligated amplified target sequence containing a first polynucleotide sequence derived from the first target polynucleotide, and the number of a second adaptor-ligated amplified target sequence containing a second polynucleotide sequence derived from the second target polynucleotide.

In some embodiments, methods for detecting a plurality of polynucleotides in a sample further comprise sequencing the plurality of adaptor-ligated amplified target sequences.

Optionally, the sequencing comprises a massively parallel sequencing procedure or a gel electrophoresis procedure.

In some embodiments, methods for detecting a plurality of polynucleotides in a sample further comprise comparing the number of plurality of adaptor-ligated amplified target sequences containing a first polynucleotide sequence derived from the first target polynucleotide with the number of plurality of adaptor-ligated amplified target sequences containing a second polynucleotide sequence derived from the second target polynucleotide.

In some embodiments, methods for detecting a plurality of polynucleotides in a sample further comprise determining the relative abundance of adaptor-ligated amplified target sequences containing the first polynucleotide sequence derived from the first target polynucleotide relative to the number of adaptor-ligated amplified target sequences containing the second polynucleotide sequence derived from the second target polynucleotide.

In some embodiments, methods for detecting a plurality of polynucleotides in a sample further comprise calculating a ratio of the number of adaptor-ligated amplified target sequences containing the first polynucleotide sequence derived from the first polynucleotide and the number of adaptor-ligated amplified target sequences containing the second polynucleotide sequence derived from the second target polynucleotide.

### DETAILED DESCRIPTION

In some embodiments, the disclosure relates generally to methods, compositions, systems, apparatuses and kits for characterizing a population of polynucleotides containing polynucleotide sequences of interest. The polynucleotide population can, for example, include or be derived from a genome, whole transcriptome, or derived from a portion of a genome or whole transcriptome. In some embodiments, the disclosed methods (and related compositions, kits and systems and apparatuses) include generating a plurality of amplicons having sequences derived from RNA. In some embodiments, the plurality of amplicons can be characterized. In some embodiments, the plurality of amplicons can be generated by amplifying a plurality of target polynucleotides in a single reaction mixture. The plurality of polynucleotides can be extracted or otherwise derived from a biological sample including cells, tissue, stool, blood, lymph, plasma, serum or other bodily fluid. In some embodiments, the plurality of polynucleotides includes a transcriptome. In some embodiments, the plurality of polynucleotides is derived from a mixed sample (e.g., includes DNA from different individuals, tissue types or from a mixture of tumor and normal cells). In some embodiments, the plurality of polynucleotides includes a mixture of maternal and fetal DNA and/or RNA. In some embodiments, the plurality of polynucleotides include circulating DNA, e.g., circulating cell-free DNA (ccf-DNA), or circulating RNA (e.g., circulating cell free RNA) present in blood or plasma. In some embodiments, the ccf-DNA (or RNA) includes a mixture of maternal and fetal DNA (or RNA). In some embodiments, the ccf-DNA (or RNA) includes a mixture of DNA (or RNA) derived from tumor and non-tumor cells from a single individual. In some embodiments, the single reaction mixture contains a plurality of target-specific primer pairs. In some embodiments, each of the primer pairs in the plurality of different target-specific primer pairs hybridizes to a different target polynucleotide. In some embodiments, the plurality of amplicons can be characterized using any procedure including: hybridizing or sequencing the plurality of amplicons; detecting the presence of one or more sequences of interest; or determining the abundance of one or more sequences of interest in the reaction mixture. Optionally, the methods can include estimating the abundance of the one or more sequences of interest in the biological sample from which the plurality of polynucleotides were extracted or otherwise derived. In some embodiments, the methods can include comparing the abundance of a first polynucleotide sequence of interest to the abundance of a second polynucleotide sequence of interest, where the first and second polynucleotides are in the same reaction mixture or in different reaction mixtures. In some embodiments, the methods can include comparing the abundance of a first polynucleotide sequence of interest to the abundance of a second polynucleotide sequence of interest, where the first and second polynucleotides are in the same biological sample or in different biological samples. In some embodiments, the methods can include comparing the relative abundance of polynucleotides derived from different chromosomes. In some embodiments, the methods can include analyzing sequence data to determine the presence of a copy number variation, e.g., within a target sequence of interest. In some embodiments, the methods can include analyzing sequence data to determine the presence of one or more chromosomal aneuploidies.

In some embodiments, the disclosure relates generally to methods, compositions, systems, apparatuses and kits for detecting a plurality of polynucleotides, comprising: (a) contacting, within a single reaction mixture, a plurality of target polynucleotides with a plurality of target-specific primer pairs under nucleic acid hybridization conditions such that different target-specific primer pair hybridizes to different target polynucleotides and only at most a single pair of target-specific primers is hybridized to any given target polynucleotide; (b) extending the target-specific primer pairs in a template-dependent fashion and forming a plurality of extension products, the extension products containing a sequence derived from a target polynucleotide; and (c) detecting the plurality of extension products. In some embodiments, the plurality of extension products includes a sequence derived from a target polynucleotide and a sequence derived from at least one target-specific primer of a primer pair. In some embodiments, the plurality of extension products comprises a plurality of amplicons. In some embodiments, at least some of the plurality of target polynucleotides are extracted or otherwise derived from a biological sample containing at least one cell or bodily fluid. In some embodiments, at least one, some or all of the plurality of the target polynucleotides are separately hybridized to only a single pair of target-specific primers. In some embodiments, at least 10%, 20%, 30%, 40%, 50%, 60%, 70%, 80%, 90%, 95% or more than 95% of the target polynucleotides are each independently and separately hybridized to a single target-specific primer pair within the reaction mixture. Optionally, the reaction mixture includes at least 10, 50, 100, 250, 500, 1000, 5000, 10,000, 15,000, 25,000, 50,000, 100,000, or 500,000 different target polynucleotides. In some embodiments, the reaction mixture includes at least 10, 50, 100, 250, 500, 1000, 5000, 10,000, 15,000, 25,000, 50,000, 100,000, or 500,000 different target-specific primer pairs. Optionally, at least one of the plurality of target-specific primers is a tailed primer. Optionally, at least one of the plurality of target-specific primers is a non-tailed primer (see U.S. published Application No. 2105/0344938, which is incorporated by reference in its entirety). Optionally, the reaction mixture includes a single pair of target-specific primers configured to hybridize with each different target polynucleotide of the plurality of target polynucleotides. Optionally, the plurality of target polynucleotides is derived from a sample. In some embodiments, the detecting includes sequencing at least a portion of the extension products (e.g., amplicons). In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides, comprising: (a) contacting, within a single reaction mixture, a plurality of target polynucleotides with a plurality of target-specific primer pairs under nucleic acid hybridization conditions such that each different target-specific primer pair hybridizes to different target polynucleotides and only at most a single pair of target-specific primers is hybridized to any given target polynucleotide; (b) extending the target-specific primer pairs in a template-dependent fashion and forming a plurality of amplicons, each amplicon containing a sequence derived from a target polynucleotide; and (c) detecting the amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, at least one, some or all of the plurality of the target polynucleotides are separately hybridized to only a single pair of target-specific primers. In some embodiments, at least 10%, 20%, 30%, 40%, 50%, 60%, 70%, 80%, 90%, 95% or more than 95% of the target polynucleotides are each independently and separately hybridized to a single target-specific primer pair within the reaction mixture. Optionally, the reaction mixture includes at least 10, 50, 100, 250, 500, 1000, 5000, 10,000, 15,000, 25,000, 50,000, 100,000, or 500,000 different target polynucleotides. In some embodiments, the reaction mixture includes at least 10, 50, 100, 250, 500, 1000, 5000, 10,000, 15,000, 25,000, 50,000, 100,000, or 500,000 different target-specific primer pairs. Optionally, the reaction mixture includes a single pair of target-specific primers configured to hybridize with each different target polynucleotide of the plurality of target polynucleotides. Optionally, the plurality of target polynucleotides is derived from a sample. In some embodiments, the detecting includes sequencing at least a portion of the amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) contacting, within a single reaction mixture, a plurality of target polynucleotides derived from a sample with a plurality of target-specific primer pairs under nucleic acid hybridization conditions such that at least some of the target-specific primer pairs hybridize to at least some of the target polynucleotides and at least one of the target polynucleotides is hybridized to no more than one primer pair; (b) extending the target-specific primer pairs in a template-dependent fashion and forming a plurality of amplicons, each amplicon containing a sequence derived from a target polynucleotide; and (c) detecting the plurality of amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, at least one, some or all of the plurality of the target polynucleotides are separately hybridized to only a single pair of target-specific primers. In some embodiments, at least 10%, 20%, 30%, 40%, 50%, 60%, 70%, 80%, 90%, 95% or more than 95% of the target polynucleotides are each independently and separately hybridized to a single target-specific primer pair within the reaction mixture. Optionally, the reaction mixture includes at least 10, 50, 100, 250, 500, 1000, 5000, 10,000, 15,000, 25,000, 50,000, 100,000, or 500,000 different target polynucleotides. In some embodiments, the reaction mixture includes at least 10, 50, 100, 250, 500, 1000, 5000, 10,000, 15,000, 25,000, 50,000, 100,000, or 500,000 different target-specific primer pairs. Optionally, the reaction mixture includes a single pair of target-specific primers configured to hybridize with each different target polynucleotide of the plurality of target polynucleotides. In some embodiments, the detecting includes sequencing at least a portion of the amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) contacting, within a single reaction mixture, a plurality of target polynucleotides derived from a sample with a plurality of target-specific primer pairs under nucleic acid hybridization conditions such that different target-specific primer pairs hybridize to different target polynucleotides and only a single pair of target-specific primers hybridize to any given target polynucleotide; (b) extending the target-specific primer pairs in a template-dependent fashion and forming a plurality of amplicons, each amplicon containing a sequence derived from a target polynucleotide; and (c) detecting the plurality of amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, at least one, some or all of the plurality of the target polynucleotides are separately hybridized to only a single pair of target-specific primers. In some embodiments, at least 10%, 20%, 30%, 40%, 50%, 60%, 70%, 80%, 90%, 95% or more than 95% of the target polynucleotides are each independently and separately hybridized to a single target-specific primer pair within the reaction mixture. Optionally, the reaction mixture includes at least 10, 50, 100, 250, 500, 1000, 5000, 10,000, 15,000, 25,000, 50,000, 100,000, or 500,000 different target polynucleotides. In some embodiments, the reaction mixture includes at least 10, 50, 100, 250, 500, 1000, 5000, 10,000, 15,000, 25,000, 50,000, 100,000, or 500,000 different target-specific primer pairs. Optionally, the reaction mixture includes a single pair of target-specific primers configured to hybridize with each different target polynucleotide of the plurality of target polynucleotides. In some embodiments, the detecting includes sequencing at least a portion of the amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides, comprising: (a) contacting, within a single reaction mixture, a plurality of target polynucleotides with a plurality of target-specific primer pairs under nucleic acid hybridization conditions such that different target-specific primer pairs hybridize to different target polynucleotides and at least some of the target polynucleotides are hybridized to no more than one pair of target-specific primers; (b) extending the target-specific primer pairs in a template-dependent fashion and forming a plurality of amplicons, each amplicon containing a sequence derived from a target polynucleotide; and (c) detecting the amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, at least one, some or all of the plurality of the target polynucleotides are separately hybridized to only a single pair of target-specific primers. In some embodiments, at least 10%, 20%, 30%, 40%, 50%, 60%, 70%, 80%, 90%, 95% or more than 95% of the target polynucleotides are each independently and separately hybridized to a single target-specific primer pair within the reaction mixture. Optionally, the reaction mixture includes at least 10, 50, 100, 250, 500, 1000, 5000, 10,000, 15,000, 25,000, 50,000, 100,000, or 500,000 different target polynucleotides. In some embodiments, the reaction mixture includes at least 10, 50, 100, 250, 500, 1000, 5000, 10,000, 15,000, 25,000, 50,000, 100,000, or 500,000 different target-specific primer pairs. Optionally, the reaction mixture includes a single pair of target-specific primers configured to hybridize with each different target polynucleotide of the plurality of target polynucleotides. In some embodiments, the detecting includes sequencing at least a portion of the amplicons. In some embodiments, when a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) contacting, within a single reaction mixture, (i) a plurality of target-specific primer pairs, with (ii) a plurality of target polynucleotides derived from a sample, where the contacting is performed under nucleic acid hybridization conditions such that different target-specific primer pairs hybridize to different target polynucleotides, where the plurality of target polynucleotides contains at least a first and a second target polynucleotide, and a first target-specific primer pair hybridizes to the first target polynucleotide and a second target-specific primer pair hybridizes to the second target polynucleotide; (b) extending the target-specific primer pairs in a template-dependent fashion and forming a plurality of amplicons, where the extending includes extending the first target-specific primer pair in a template-dependent fashion and forming a plurality of first amplicons, where the first amplicons contain a sequence derived from the first target polynucleotide, and where the extending includes extending the second target-specific primer pair in a template-dependent fashion and forming a plurality of second amplicons, where the second amplicons contain a sequence derived from the second target polynucleotide; and (c) detecting at least the first and the second amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, at least one of the target polynucleotides hybridizes to only a single pair of target-specific primers. In some embodiments, the first target polynucleotide hybridizes only to the first target-specific primer pair and not to any other target-specific primers in the reaction mixture. In some embodiments, the second target polynucleotide hybridizes only to the second target-specific primer pair and not to any other target-specific primers in the reaction mixture. In some embodiments, the detecting includes sequencing at least a portion of the amplicons. In some embodiments, when a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) contacting, within a single reaction mixture, (i) a plurality of different target-specific primer pairs, with (ii) a plurality of target polynucleotides, where the contacting is performed under nucleic acid hybridization conditions such that the plurality of different target-specific primer pairs hybridizes to different target polynucleotides, wherein the single reaction mixture includes 10, 50, 100, 250, 500, 1000, 5000, 10,000, 15,000, 25,000, 50,000, 100,000, or 500,000 different target specific primer pairs; (b) extending the target-specific primer pairs in a template-dependent fashion and forming a plurality of amplicons, where each amplicon includes a sequence derived from a target polynucleotide and at least one of the target-specific primer pairs; and (c) detecting the plurality of amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, at least one of the target polynucleotides hybridizes to only a single pair of target-specific primers. In some embodiments, at least some of the target polynucleotides hybridize to a single corresponding target-specific primer pair and not to any other target-specific primers in the reaction mixture.

In some embodiments, the detecting includes sequencing at least a portion of the amplicons. In some embodiments, when a single primer pair is used to generate a single sequence for each target polynucleotide, a sequence read assembly is not performed.

In some embodiments, the plurality of target polynucleotides in the reaction mixture includes a population of genomic DNA or RNA extracted directly from a biological sample. The biological sample can include cells, tissue, stool, lymph, blood, plasma, serum, cerebrospinal fluid, cell or tissue exudate or other bodily fluid.

In some embodiments, the plurality of target polynucleotides in the reaction mixture includes a population of polynucleotides derived from such total genomic DNA or total RNA. For example, the plurality of target polynucleotides can include specific sequences derived via reverse transcription and/or selective or non-selective amplification of such total genomic DNA or RNA.

In some embodiments, the plurality of target polynucleotides in the reaction mixture can be the products of additional manipulations such as restriction digestion, fragmentation, end polishing and/or adapter ligation, or any combination of the foregoing.

In some embodiments, the plurality of target polynucleotides in the reaction mixture includes a population of DNA fragments substantially representing an entire genome or any portion thereof.

In some embodiments, the plurality of target polynucleotides in the reaction mixture includes a population of cDNA fragments derived from RNA transcripts and substantially representing an entire transcriptome or any portion thereof.

Optionally, the plurality of target-specific primers includes at least one target-specific primer pair for each different DNA or cDNA fragment present (or expected to be present) in the reaction mixture.

Optionally, the plurality of target-specific primers includes only a single target-specific primer pair for each different DNA or cDNA fragment present (or expected to be present) in the reaction mixture.

In some embodiments, includes 10, 50, 100, 250, 500, 1000, 5000, 10,000, 15,000, 25,000, 50,000, 100,000, 500,000, or 1,000,000 different DNA or cDNA fragments and about the same number of different target specific primer pairs.

In some embodiments, the disclosure relates generally to methods, compositions, systems, apparatuses and kits for detecting a plurality of polynucleotides in a sample, comprising: (a) contacting, within a single reaction mixture, (i) a plurality of target-specific primer pairs containing 20,000 different primer pairs, with (ii) a plurality of target polynucleotides having sequences derived from RNA from one or more cells, where the contacting is performed under nucleic acid hybridization conditions such that the 20,000 different target-specific primer pairs hybridizes to different target polynucleotides, and only a single pair of target-specific primers hybridizes to any given target polynucleotide; (b) extending the target-specific primer pairs in a template-dependent fashion and forming a plurality of amplicons, where each amplicon includes a sequence derived from a target polynucleotide and at least one of the target-specific primer pairs; and (c) detecting the plurality of amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, at least some of the target polynucleotides hybridize to a single corresponding target-specific primer pair and not to any other target-specific primers in the reaction mixture. In some embodiments, the detecting includes sequencing at least a portion of the amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) contacting, within a single reaction mixture, (i) a plurality of different target-specific primer pairs having a cleavable group, with (ii) a plurality of target polynucleotides derived from RNA from one or more cells, where the contacting is performed under nucleic acid hybridization conditions such that the plurality of different target-specific primer pairs hybridizes to different target polynucleotides, and only a single pair of target-specific primers hybridizes to any given target polynucleotide; (b) extending the target-specific primer pairs in a template-dependent fashion and forming a plurality of amplicons, where each amplicon includes a sequence derived from a target polynucleotide and a primer-derived sequence having the cleavable group; (c) cleaving the cleavable group in the primer-derived sequence to produce a cleaved amplified target sequence; (d) ligating at least one adaptor to an end of at least one cleaved amplified target sequence to produce a adaptor-ligated amplified target sequence; and (e) detecting the adaptor-ligated amplified target sequence. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, the detecting includes sequencing at least some of the adaptor-ligated amplified target sequences. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) generating a plurality of target polynucleotides having sequences derived from a plurality of RNA in a sample, by reverse-transcribing the plurality of RNA with a plurality of primers to produce the plurality of target polynucleotides; (b) contacting, within a single reaction mixture, a plurality of target polynucleotides derived from a sample with a plurality of target-specific primer pairs under nucleic acid hybridization conditions such that different target-specific primer pairs hybridize to different target polynucleotides and only a single pair of target-specific primers hybridize to any given target polynucleotide; (c) extending the target-specific primer pairs in a template-dependent fashion and forming a plurality of amplicons, each amplicon containing a sequence derived from a target polynucleotide; and (d) detecting the plurality of amplicons. Optionally, the plurality of RNA includes RNA sequences present in a biological sample. The plurality of RNA sequences includes different RNA sequences. Optionally, the plurality of RNA represents total RNA, or a portion of total RNA, from a biological sample. Optionally, the plurality of RNA sequences includes a transcriptome derived from a biological sample. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, the detecting includes sequencing at least a portion of the amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) generating a plurality of target polynucleotides having sequences derived from a plurality of RNA in a sample, by reverse-transcribing the plurality of RNA with a plurality of primers to produce the plurality of target polynucleotides; (b) contacting, within a single reaction mixture, (i) a plurality of target-specific primer pairs, with (ii) a plurality of target polynucleotides derived from a sample, where the contacting is performed under nucleic acid hybridization conditions such that different target-specific primer pairs hybridize to different target polynucleotides, where the plurality of target polynucleotides contains at least a first and a second target polynucleotide, and a first target-specific primer pair hybridizes to the first target polynucleotide and a second target-specific primer pair hybridizes to the second target polynucleotide; (c) extending the target-specific primer pairs in a template-dependent fashion and forming a plurality of amplicons, where the extending includes extending the first target-specific primer pair in a template-dependent fashion and forming a plurality of first amplicons, where the first amplicons contain a sequence derived from the first target polynucleotide, and where the extending includes extending the second target-specific primer pair in a template-dependent fashion and forming a plurality of second amplicons, where the second amplicons contain a sequence derived from the second target polynucleotide; and (d) detecting at least the first and the second amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, the detecting includes sequencing at least some of the amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) generating a plurality of target polynucleotides having sequences derived from a plurality of RNA in a sample, by reverse-transcribing the plurality of RNA with a plurality of primers to produce the plurality of target polynucleotides; (b) contacting, within a single reaction mixture, (i) a plurality of different target-specific primer pairs, with (ii) a plurality of target polynucleotides derived from RNA from one or more cells, where the contacting is performed under nucleic acid hybridization conditions such that the plurality of different target-specific primer pairs hybridizes to different target polynucleotides, and only a single pair of target-specific primers hybridizes to any given target polynucleotide, wherein the single reaction mixture includes 100-100,000 target specific primer pairs; (c) extending the target-specific primer pairs in a template-dependent fashion and forming a plurality of amplicons, where each amplicon includes a sequence derived from a target polynucleotide and at least one of the target-specific primer pairs; and (d) detecting at least a sub-population of the amplicons. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, the detecting includes sequencing at least some of the amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) generating a plurality of target polynucleotides having sequences derived from a plurality of RNA in a sample, by reverse-transcribing the plurality of RNA with a plurality of primers to produce the plurality of target polynucleotides; (b) contacting, within a single reaction mixture, (i) a plurality of target-specific primer pairs containing at least 20,000 different primer pairs, with (ii) the plurality of target polynucleotides, where the contacting is performed under nucleic acid hybridization conditions such that the at least 20,000 different target-specific primer pairs hybridizes to different target polynucleotides, and only a single pair of target-specific primers hybridizes to any given target polynucleotide; (c) extending the target-specific primer pairs in a template-dependent fashion and forming a plurality of amplicons, where the plurality of amplicons include a sequence derived from a target polynucleotide and a sequence derived from at least one of the target-specific primer pairs; and (d) detecting the plurality of amplicons. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, a single pair of target-specific primers hybridizes to any one of at least 20,000 different target polynucleotide sequences. In some embodiments, the reverse transcribing is conducted with a plurality of random sequence primers. In some embodiments, at least some of the plurality of target polynucleotides are extracted or otherwise derived from a biological sample containing at least one cell or bodily fluid. In some embodiments, the detecting includes sequencing at least some of the plurality of amplicons. In some embodiments, since a single primer pair is used to generate a single sequence for each target polynucleotide, a sequence read assembly is not performed.

In some embodiments, the disclosure relates generally to methods, compositions, systems, apparatuses and kits for detecting a plurality of polynucleotides in a sample, comprising: (a) generating a plurality of target polynucleotides having sequences derived from a plurality of RNA in a sample, by reverse-transcribing the plurality of RNA with a plurality of primers to produce the plurality of target polynucleotides; (b) contacting, within a single reaction mixture, (i) a plurality of different target-specific primer pairs having a cleavable group, with (ii) the plurality of target polynucleotides derived from the RNA, where the contacting is performed under nucleic acid hybridization conditions such that the plurality of different target-specific primer pairs hybridizes to different target polynucleotides, and only a single pair of target-specific primers hybridizes to any given target polynucleotide; (c) extending the target-specific primer pairs in a template-dependent fashion and forming a plurality of amplicons, where each amplicon includes a sequence derived from a target polynucleotide and a primer-derived sequence having the cleavable group; and (d) cleaving the cleavable group in the primer-derived sequence to produce a cleaved amplified target sequence; (e) ligating at least one adaptor to an end of at least one cleaved amplified target sequence to produce a adaptor-ligated amplified target sequence; and (f) detecting the adaptor-ligated amplified target sequence. In some embodiments, at least some of the plurality of target polynucleotides are extracted or otherwise derived from a biological sample containing at least one cell or bodily fluid. In some embodiments, at least one of the plurality of target-specific primers is a tailed primer. In some embodiments, at least one of the plurality of target-specific primers is a non-tailed primer (see U.S. published Application No. 2105/0344938, which is incorporated by reference in its entirety). In some embodiments, the detecting includes sequencing at least some of the adaptor-ligated amplified target sequences. In some embodiments, since a single primer pair is used to generate a single sequence for each target polynucleotide, a sequence read assembly is not performed.

In some embodiments, the sample includes RNA, DNA or cDNA derived from one or more cells.

In some embodiments, at least some of the plurality of target polynucleotides are extracted or otherwise derived from a biological sample containing at least one cell or bodily fluid.

In some embodiments, one primer of at least one target-specific primer pair hybridizes to a sequence that is complementary to the sequence of any give target polynucleotide.

In some embodiments, detecting a plurality of polynucleotides in a sample further comprises: determining an amount of amplicons containing a sequence derived from a first target polynucleotide. Optionally, the determining includes counting a number of amplicons derived from the first target polynucleotide. Optionally, at least a portion of the amplicons is analyzed to count the number of amplicons derived from the first target polynucleotide. In some embodiments, the first target polynucleotide is present within, or derived from, a first chromosome.

In some embodiments, detecting a plurality of polynucleotides in a sample further comprises: determining an amount of amplicons containing a sequence derived from a second target polynucleotide. Optionally, the determining includes counting a number of amplicons derived from the second target polynucleotide. Optionally, at least a portion of the amplicons is analyzed to count the number of amplicons derived from the second target polynucleotide. In some embodiments, the second target polynucleotide is present within, or derived from, a second chromosome.

In some embodiments, detecting a plurality of polynucleotides in a sample further comprises: quantifying the number of amplicons containing a sequence derived from a first target polynucleotide. In some embodiments, the quantifying includes counting the number of amplicons containing a polynucleotide sequence of interest (e.g., a first polynucleotide sequence) that is derived from the first target polynucleotide.

In some embodiments, detecting a plurality of polynucleotides in a sample further comprises: quantifying the number of amplicons containing sequence derived from a second target polynucleotide. In some embodiments, the quantifying includes counting the number of amplicons containing a polynucleotide sequence of interest (e.g., a second polynucleotide sequence) that is derived from the second target polynucleotide. In some embodiments, the second target polynucleotide is present within, or derived from, a second chromosome. Optionally, the first and second chromosomes are different.

In some embodiments, detecting a plurality of polynucleotides in a sample further comprises: quantifying the amount of the first target polynucleotide and the amount of the second target polynucleotide present in the sample.

In some embodiments, the sample includes RNA or cDNA extracted or otherwise derived from the biological sample.

In some embodiments, the single reaction mixture includes 10, 50, 100, 250, 500, 1000, 5000, 10,000, 15,000, 25,000, 50,000, 100,000, 500,000, or 1,000,000 different primer pairs. In some embodiments, the single reaction mixture includes 10, 50, 100, 250, 500, 1000, 5000, 10,000, 15,000, 25,000, 50,000, 100,000, 500,000, or 1,000,000 different DNA or cDNA fragments and about the same number of different target specific primer pairs.

In some embodiments, the plurality of target-specific primer pairs includes 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target-specific primer pairs.

In some embodiments, the forming step further includes forming a plurality of amplicons containing sequences derived from 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target polynucleotides.

In some embodiments, detecting a plurality of polynucleotides in a sample further comprises: quantifying the number of amplicons containing sequence derived from each of the 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target polynucleotides.

In some embodiments, the sample includes nucleic acids (e.g., RNA, DNA or cDNA) derived from one or more cells and the method (and related compositions, systems, apparatuses and kits) includes quantifying the amounts for each of 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different nucleic acids present in the sample.

In some embodiments, the sample includes cDNA derived from RNA (e.g., total cellular RNA) and the method (and related compositions, systems, apparatuses and kits) includes quantifying the amounts for each of 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different transcripts present in the sample.

In some embodiments, the method further comprises re-amplifying the plurality of amplicons.

In some embodiments, the method further comprises calculating a ratio of the number of amplicons derived from the first target polynucleotide, and the number of amplicons derived from the second target polynucleotide.

In some embodiments, the reverse transcribing can be conducted with a plurality of random sequence primers, target-specific primers, or polyT primers.

In some embodiments, the reverse transcribing can be conducted by directly ligating the RNA to a plurality of double-stranded RNA/DNA or DNA/DNA adaptors, heating to remove one strand of the double-stranded adaptors, and conducting a reverse transcription reaction with primers that hybridize at least one adaptor sequence. In some embodiments, the reverse transcribing can be conducted according to an RNA-Seq procedure described in U.S. Pat. No. 8,192,941, which is incorporated by reference in its entirety.

In some embodiments, at least one of the primers from the pairs of the target-specific primers includes a cleavable group.

In some embodiments, the disclosure relates generally to methods, compositions, systems, apparatuses and kits, comprising conducting a multiplex nucleic acid amplification reaction on target polynucleotide sequences that represent RNA or DNA. For example, the target polynucleotide sequences that represent RNA include cDNA sequences derived from a whole transcriptome, or from a portion of a whole transcriptome. In some embodiments, the multiplex nucleic acid amplification reaction can be performed after any procedure that converts RNA to a plurality of cDNA. In some embodiments, the target polynucleotides (e.g., plurality of DNA) can be produced in any reverse transcription reaction. In some embodiments, the target polynucleotides can be subjected to a multiplex nucleic acid amplification reaction to produce a plurality of amplicons having sequences derived from RNA. In some embodiments, the multiplex nucleic acid amplification reaction uses a plurality of target-specific primer pairs.

In some embodiments, the disclosure relates generally to methods, compositions, systems, apparatuses and kits, comprising conducting a multiplex nucleic acid amplification reaction with a plurality of target polynucleotides and a plurality of target-specific primer pairs in a single reaction mixture to produce a plurality of different amplicons.

In some embodiments, the disclosure relates generally to methods, compositions, systems, apparatuses and kits for amplifying a plurality of target polynucleotides to produce a plurality of amplicons. In some embodiments, the plurality of amplicons can be generated by amplifying the plurality of target polynucleotides with a plurality of target-specific primer pairs in a single amplification mixture.

In some embodiments, the disclosure relates generally to methods, compositions, systems, apparatuses and kits for amplifying one or more target polynucleotides within a sample containing a plurality of different target polynucleotides. Optionally, a plurality of different target polynucleotides, for example at least 500, 1000, 2000, 2500, 5000, 7500, 10000, 15000, 20000, 25000, 50000, 100000, 200000, 400000 or 500000, are amplified within a single amplification reaction.

In some embodiments, the disclosure relates generally to methods, compositions, systems, apparatuses and kits, comprising a reaction mixture. In some embodiments, the reaction mixture contains a single type of nucleic acid or a mixture of different types of nucleic acids. In some embodiments, the reaction mixture contains a plurality of nucleic acids having the same sequence or different sequences. In some embodiments, the reaction mixture contains single-stranded or double-stranded nucleic acids. In some embodiments, the sample contains RNA, cDNA or DNA. In some embodiments, the reaction mixture contains a plurality of nucleic acids that are naturally-occurring, recombinant or synthetically-prepared. In some embodiments, the reaction mixture contains nucleic acids that are isolated from a single fresh or archived cell, fresh cells, fresh tissues, or archived cells or tissues that are formalin-treated and/or embedded in paraffin or plastic, or cells or tissues that are formalin fixed paraffin-embedded (FFPE). In some embodiments, the reaction mixture contains nucleic acids that are isolated from any source including from organisms such as prokaryotes, eukaryotes (e.g., humans, plants and animals), fungus, and viruses; cells; tissues; normal or diseased cells or tissues or organs, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods.

In some embodiments, the reaction mixture contains nucleic acids that are unfragmented, or fragmented by mechanical force, chemical, enzyme or heat. In some embodiments, the reaction mixture contains nucleic acids that are depleted of, or enriched for, one or more nucleic acid species.

In some embodiments, the reaction mixture includes polynucleotides derived from whole-genome amplification (WGA) of genomic DNA extracted from a single cell, multiple cells, whole tissue, blood or other bodily fluid. Optionally, the single cell is taken from a fertilized zygote, blastocyst or embryo, or is a fetal cell extracted from maternal tissue or blood, or is a tumor cell (e.g., a circulating tumor cell).

In some embodiments, the disclosure relates generally to methods (as well as related compositions, systems, apparatus and kits) for performing multiplex amplification of target polynucleotides. In some embodiments, the method includes amplifying a plurality of target polynucleotides within a single reaction mixture including two or more target polynucleotides. Optionally, multiple different target polynucleotides of interest can be amplified in a single reaction mixture using one or more target-specific primers in the presence of a polymerase under amplification conditions to produce a plurality of different target amplicons. The amplifying optionally includes contacting a nucleic acid molecule including at least one target polynucleotide with one or more target-specific primers and at least one polymerase under amplification conditions, thereby producing one or more target amplicons. Optionally, at least one of the target-specific primers includes a cleavable group. Optionally, the cleavable group is cleavable with uracil DNA glycosylase (UDG, also referred to as UNG), formamidopyrimidine DNA glycosylase (Fpg), or a FuPa reagent.

In some embodiments, the disclosure relates generally to methods, compositions, systems, apparatuses and kits for amplifying a plurality of target polynucleotides comprising contacting a plurality of target polynucleotides with a plurality of target-specific primer pairs. In some embodiments, the plurality of target polynucleotides and the plurality of target-specific primer pairs are contacted together in a single reaction mixture. Optionally, at least one of the target-specific primers in the plurality of target-specific primer pairs includes a cleavable group. Optionally, the cleavable group is cleavable with uracil DNA glycosylase (UDG, also referred to as UNG), formamidopyrimidine DNA glycosylase (Fpg), or a FuPa reagent.

In some embodiments, the single reaction mixture comprises any one or any combination of a plurality of target polynucleotides, a plurality of target-specific primer pairs, at least one polymerase, and a plurality of nucleotides. Optionally, the plurality of nucleotides includes one or more non-labeled nucleotides or at least one nucleotide labeled with a detectable moiety.

In some embodiments, the plurality of target polynucleotides are contacted with a plurality of target-specific primer pairs in a single reaction mixture, where the plurality of target-specific primer pairs contains 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target-specific primer pairs. Optionally, at least one of the target-specific primers in the plurality of target-specific primer pairs includes a cleavable group. Optionally, each target-specific primer in one or more pairs includes a cleavable group. Optionally, the cleavable group is cleavable with uracil DNA glycosylase (UDG, also referred to as UNG), formamidopyrimidine DNA glycosylase (Fpg), or a FuPa reagent.

In some embodiments, the single reaction mixture further includes RNase H to degrade any RNA that may be present.

In some embodiments, the single reaction mixture further comprises any one or any combination of: magnesium, manganese, formamide, DMSO, betaine, trehalose, spermidine, sulfones, sodium pyrophosphate, low molecular amides, and/or single-stranded binding proteins. In some embodiments, the single reaction mixture includes a plurality of target polynucleotides which comprise a plurality of single-stranded or double-stranded nucleic acids (e.g., cDNA).

In some embodiments, the plurality of target polynucleotides and the plurality of target-specific primer pairs are contacted together, in a single reaction mixture, under nucleic acid hybridization conditions so that different target-specific primer pairs hybridize to different target polynucleotides.

In some embodiments, at least one target-specific primer can hybridize under stringent conditions to at least some portion of a corresponding target polynucleotide sequence.

In some embodiments, at least one target specific primer in a target specific primer pair can include at least one sequence that is substantially complementary or substantially identical to at least a portion of a corresponding target polynucleotide sequence or its complement. In some embodiments, at least a portion of each of the different target-specific primer pairs can be substantially complementary to a target sequence in a polynucleotide.

In some embodiments, the plurality of target specific primer pairs includes at least a first and a second target specific primer pair that are different from each other. In some embodiments, the first target specific primer pair can be substantially non-complementary to another target sequence in the sample. In some embodiments, the first target specific primer pair can be substantially non-complementary to a second target polynucleotide sequence.

In some embodiments, the different target-specific primer pairs hybridize to the different target polynucleotides to form a plurality of different primer/polynucleotide complexes. In some embodiments, each primer pair in the plurality of target-specific primer pairs is configured or designed to hybridize to a different target polynucleotide sequence of interest, optionally under high-stringency hybridization conditions. In some embodiments, a single pair of target-specific primers can hybridize to one target polynucleotide sequence. Optionally, only a single pair of target-specific primers hybridize to any given target polynucleotide. Optionally, more than one pair of target-specific primers hybridize to any given target polynucleotide.

In some embodiments, each primer pair in the plurality of target-specific primer pairs is designed to hybridize to a different target polynucleotide sequence of interest. For example, if there are N different target polynucleotides sequences of interest, then the plurality of target-specific primer pairs will contain N different primer pairs. In some embodiments, the single reaction mixture can contain 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target-specific primer pairs. In some embodiments, the plurality of target-specific primer pairs includes about 20,000 different target-specific primer pairs.

In some embodiments, at least one of the target-specific primer pairs has minimal cross-hybridization with any other pair of primers in the single reaction mixture.

In some embodiments, the multiplex nucleic acid amplification reaction includes contacting a plurality of polynucleotides with a plurality of target-specific primer pairs, under suitable nucleic acid hybridization conditions. The suitable hybridization conditions can include the plurality of polynucleotides and the plurality of target-specific primer pairs in an aqueous solution containing salts (e.g., sodium), magnesium, buffers, and/or formamide. The hybridization can be conducted at a temperature that is about 5-30° C. below the melting temperature. Under the suitable hybridization condition, the different pairs of target-specific primers can hybridize to different target polynucleotides (e.g., cDNA) to form a plurality of different primer/polynucleotide complexes. In some embodiments, hybridization conditions include high-stringency hybridization conditions. For example, high-stringency conditions can include any conditions whereby duplexes only form between strands (e.g., target polynucleotide and primers) having perfect one-to-one complementarity.

In some embodiments, the disclosure relates generally to methods (as well as related compositions, systems, apparatus and kits) for performing nucleic acid synthesis of target polynucleotides. In some embodiments, the method includes synthesizing a plurality of target polynucleotides within a single reaction mixture including two or more target polynucleotides. Optionally, multiple different target polynucleotides of interest can be synthesized in a single reaction mixture using one or more target-specific primers in the presence of a catalyst (e.g., an enzyme that can catalyze the polymerization of nucleotides and nucleotides, such as dNTP's to promote extension of the one or more target-specific primers) under synthesis conditions to produce a plurality of different target amplicons. The synthesizing optionally includes contacting a nucleic acid molecule including at least one target polynucleotide with one or more target-specific primers and at least one polymerase under synthesis conditions, thereby producing one or more target amplicons.

In some embodiments, the disclosure relates generally to methods, compositions, systems, apparatuses and kits for conducting a primer extension reaction to amplify a plurality of different target polynucleotides in a the multiplex nucleic acid amplification reaction, where the plurality of different target polynucleotides are amplified substantially simultaneously in a single reaction mixture containing a plurality of different target specific primer pairs.

In some embodiments, the multiplex nucleic acid amplification reaction of the present teachings can substantially simultaneously amplify at least a first target sequence and at least a second target sequence that are less than 50% complementary to each other. In some embodiments, the first target sequence and the second target sequence are substantially non-complementary to each other. In some embodiments, at least one of the target-specific primer pairs has minimal cross-hybridization with any other pair of primers in the single reaction mixture.

In some embodiments, one or more of the methods of amplifying disclosed herein includes performing a target-specific amplification. Performing the target-specific amplification can include amplifying one or more target polynucleotides using one or more exclusively target-specific primers, i.e., primers that do not include a shared or universal sequence motif with other target-specific primers or other target polynucleotides in the reaction mixture. In some embodiments, a target polynucleotide can be amplified using no more than a single pair of target-specific primers. Typically, one or more of the target-specific primers are substantially complementary, or complementary, to at least some portion of the corresponding target polynucleotide, or to some portion of the nucleic acid molecule including the corresponding target polynucleotide. In some embodiments, one, some or all of the target-specific primers (or primer pairs) are substantially complementary, or complementary, to at least some portion of their corresponding target polynucleotide, or to some portion of the nucleic acid molecule including the corresponding target polynucleotide, across their (i.e., the target specific primers′) entire length.

In some embodiments, the plurality of target polynucleotides is amplified by conducting a primer extension reaction on the primer/polynucleotide complexes. In some embodiments, the primer extension reaction comprises incorporating one nucleotide onto a primer that is part of a primer/polynucleotide complex. In some embodiments, the nucleotide is incorporated onto the primer in a template-based manner, which can include complementary base pairing, including standard A-T or C-G base pairing, or optionally other forms of base-pairing interactions. In some embodiments, the primer extension reaction includes successively incorporating nucleotides onto a primer that is part of the primer/polynucleotide complex.

In some embodiments, the primer extension reaction includes the target-specific primer pairs, the target polynucleotides, at least one polymerase, and a plurality of nucleotides. In some embodiments, the polymerase comprises a DNA-dependent DNA polymerase. Optionally, the polymerase exhibits RNA-dependent DNA polymerase activity. In some embodiments, the plurality of nucleotides comprises unlabeled nucleotides, or at least one labeled nucleotide.

In some embodiments, the primer extension reaction can be conducted in a single reaction mixture. In some embodiments, the primer extension reaction produces at least one amplicon or a plurality of amplicons. In some embodiments, the primer extension reaction produces at least two different amplicons that include sequences that are less than 50% complementary to each other. In some embodiments, the primer extension reaction produces at least on amplicon containing a sequence having at least a portion of a target polynucleotide sequence. In some embodiments, each amplicon also includes the sequence of at least one primer of a target-specific primer pair. In some embodiments, the primer extension reaction produces at least one amplicon containing a primer-derived sequence on at least one end of the amplicon. In some embodiments, the primer-derived sequence on the at least one end of the plurality of amplicons includes at least one cleavable group. Optionally, the cleavable group comprises a modified nucleoside, nucleotide or nucleobase. Optionally, the cleavable group comprises: uracil, uridine, inosine, or 7,8-dihydro-8-oxoguanine (8-oxoG) nucleobases. Optionally, the cleavable group is cleavable with uracil DNA glycosylase (UDG, also referred to as UNG), formamidopyrimidine DNA glycosylase (Fpg), or a FuPa reagent.

In some embodiments, the disclosure relates generally to methods, compositions, systems, apparatuses and kits comprising a plurality of amplicons that are produce by any of the multiplex nucleic acid amplification reactions of the present teachings.

In another embodiment, the methods, compositions, systems, apparatuses and kits for amplifying one or more target polynucleotides in a single amplification reaction include at least two amplified target polynucleotides that are not complementary along their length to a different amplified target polynucleotide in the single reaction mixture. In another embodiment, the methods, compositions, systems, apparatuses and kits for amplifying one or more target amplicons include at least two target amplicons that are not complementary along their length to a different target amplicon in the single reaction mixture. In another embodiment, the methods, compositions, systems, apparatuses and kits for amplifying a plurality of target polynucleotides in a single amplification reaction include a plurality of target polynucleotides that are not complementary along their length to a different amplified target polynucleotide in the single reaction mixture.

In some embodiments, the amplification conditions can produce at least two different target amplicons that are less than 50% complementary to each other along their length. In some embodiments, at least one target amplicon is substantially non-complementary, or non-complementary, along its length to another target amplicon in the reaction mixture. In some embodiments, a target amplicon can be substantially non-complementary, or non-complementary, along its length to any one or more target polynucleotides in the sample that do not correspond to the target amplicon nucleic acid sequence. In another embodiment, the at least two different target amplicons are not complementary along their length to any other target amplicon in the reaction mixture. In one embodiment, the at least two different target amplicons are not complementary to another nucleic acid molecule in the amplification reaction mixture. In another embodiment, the at least two different target amplicons are not complementary along their length to any target-specific primer in the amplification reaction mixture.

In some embodiments, the multiplex nucleic acid amplification reactions produce at least one amplicon containing a sequence having at least a portion of a target polynucleotide sequence, where the target polynucleotide contains wild-type or mutant sequences, fusion sequences, spliced sequences, unspliced sequences, splice isoforms, allelic variant sequences, single nucleotide variant sequences, or cell or tissue-specific expressed sequences.

In some embodiments, the multiplex nucleic acid amplification reactions produce a first amplicon having at least a portion of a first target polynucleotide sequence, and a second amplicon having at least a portion of a second target polynucleotide sequence. In some embodiments, the sequences of the first and the second amplicons are substantially non-complementary to each other. In some embodiments, the multiplex nucleic acid amplification reactions produce at least two different amplicons that include sequences that are less than 50% complementary to each other. In some embodiments, the multiplex nucleic acid amplification reactions produce at least one amplicon containing a sequence having at least a portion of a target polynucleotide sequence, and the at least one amplicon also includes the sequence of at least one primer (a primer-derived sequence) of a target-specific primer pair. In some embodiments, the multiplex nucleic acid amplification reactions produce at least one amplicon containing a primer-derived sequence on at least one end of the amplicon.

In some embodiments, the primer-derived sequence on the at least one end of the plurality of amplicons includes at least one cleavable group. Optionally, the cleavable group comprises a modified nucleoside, nucleotide or nucleobase. Optionally, the cleavable group comprises: uracil, uridine, inosine, or 7,8-dihydro-8-oxoguanine (8-oxoG) nucleobases. Optionally, the cleavable group is cleavable with uracil DNA glycosylase (UDG, also referred to as UNG), formamidopyrimidine DNA glycosylase (Fpg), or a FuPa reagent.

In some embodiments, a nucleic acid molecule in a sample, an amplified target polynucleotide, an adapter or a target-specific primer includes a 5′ end and a 3′ end. The 5′ end can include a free 5′ phosphate group or its equivalent; the 3′ end can include a free 3′ hydroxyl group or its equivalent. Optionally, the ends of an amplified target polynucleotide can be non-complementary to the ends of another amplified target polynucleotide in the reaction mixture. In some embodiments, the 3′ end can include about 30 nucleotides, or about 15 nucleotides, or about 10 nucleotides, or about 8 nucleotides from the 3′ hydroxyl group. In some embodiments, the 5′ end can include about 30 nucleotides, or about 15 nucleotides, about 10 nucleotides, or about 8 nucleotides from the 5′ phosphate group. In some embodiments, any one amplified target polynucleotide having a 3′ end and a 5′ end can be substantially non-complementary, or non-complementary, to any portion of any other amplified target polynucleotide in the reaction mixture. Having a plurality of target polynucleotides with substantially non-complementary, or non-complementary, 3′ and 5′ ends within the reaction mixture dramatically and significantly reduces the formation of spurious artifacts, such as primer dimers and non-specific priming.

In some embodiments, the amplicons can be phosphorylated. In some embodiments, phosphorylation of the amplicons can be conducted using a FuP reagent. In some embodiments, the FuP reagent can include a DNA polymerase, a DNA ligase, at least one uracil cleaving or modifying enzyme, and/or a storage buffer. In some embodiments, the FuP reagent can further include at least one of the following: a preservative and/or a detergent.

In some embodiments, phosphorylation of the amplicons can be conducted using a FuPa reagent. In some embodiments, the FuPa reagent can include a DNA polymerase, at least one uracil cleaving or modifying enzyme, an antibody and/or a storage buffer. In some embodiments, the FuPa reagent can further include at least one of the following: a preservative and/or a detergent. In some embodiments, the antibody is provided to inhibit the DNA polymerase and 3′-5′ exonuclease activities at ambient temperature.

In some embodiments, the disclosure relates generally to methods for performing amplification of a target polynucleotide or target amplicon (as well as related compositions, systems, apparatuses and kits using the disclosed methods) and can include a digestion step. In some embodiments, the methods also include a ligating step, and the digestion step is performed prior to the ligating step. In some embodiments, an amplified target polynucleotide can be partially digested prior to performing the ligation step. For example, an amplified target polynucleotide can be digested by enzymatic, thermal, chemical, or other suitable means. In some embodiments, an amplified target polynucleotide can be digested prior to the ligating to produce a blunt-end or sticky-ended amplified target polynucleotide. In some embodiments, a blunt-ended amplified target polynucleotide can include a 5′ phosphate group at the 5′ end of the digested amplified target polynucleotide. In some embodiments, a blunt-ended amplified target amplicon can include a 5′ phosphate group at the 5′ end of the digested amplified target amplicon.

In some embodiments, a target-specific primer, adapter, target amplicon, amplified target polynucleotide or nucleic acid molecule can include one or more cleavable moieties, also referred to herein as cleavable groups. Optionally, the methods can further include cleaving at least one cleavable group of the target-specific primer, adapter, target amplicon, amplified target polynucleotide or nucleic acid molecule. In some embodiments, the cleaving can be performed before or after any of the other steps of the disclosed methods. In some embodiments, the cleavage step occurs after the amplifying and prior to a ligating step. In one embodiment, the cleaving includes cleaving at least one amplified target polynucleotide or target amplicon prior to the ligating. In some embodiments, the cleaving can include cleaving at least 50%, at least 60%, at least 70%, at least 80%, at least 90%, at least 95%, or more of the target-specific primers present in the single reaction mixture. In some embodiments, the cleavable moiety can be present as a modified nucleotide, nucleoside or nucleobase. In some embodiments, the cleavable moiety can include a nucleobase not naturally occurring in the target sequence of interest. For example, uracil or uridine can be incorporated into a DNA as a cleavable group. In one exemplary embodiment, a uracil DNA glycosylase can be used to cleave the cleavable group from a nucleic acid including uracil. In another embodiment, inosine can be incorporated into a DNA-based nucleic acid as a cleavable group. In one exemplary embodiment, EndoV can be used to cleave near the inosine residue and a further enzyme, such as Klenow, can be used to create blunt-ended fragments capable of blunt-ended ligation. In another exemplary embodiment, the enzyme hAAG can be used to cleave inosine residues from a nucleic acid creating abasic sites that can be further processed by one or more enzymes, such as Klenow, to create blunt-ended fragments capable of blunt-ended ligation. In another embodiment, the cleavable moiety can include an enzymatic restriction recognition sequence, such as the Hind III, Spel, HpaI or DpnII, located within the nucleic acid sequence of the target polynucleotide, amplicon, or target-specific primer.

In some embodiments, the disclosure relates generally to methods, compositions, systems, apparatuses and kits, comprising conducting a multiplex nucleic acid amplification reaction and further comprising a cleaving step.

In some embodiments, the plurality of amplicons comprises a primer-derived sequence on at least one end of the amplicon, and the primer-derived sequence contains a cleavable group. In some embodiments, the cleavable group comprises: uracil, uridine, inosine, or 7,8-dihydro-8-oxoguanine (8-oxoG) nucleobases.

In some embodiments, the primer-derived sequence (which contains a cleavable group) on at least one end of the plurality of amplicons can be cleaved with a cleaving agent. Optionally, the cleaving agent comprises uracil DNA glycosylase (UDG, also referred to as UNG), formamidopyrimidine DNA glycosylase (Fpg), or a FuPa reagent. Optionally, EndoV can be used to cleave near the inosine residue and a further enzyme such as Klenow can be used to create blunt-ended fragments capable of blunt-ended ligation. Optionally, the enzyme hAAG can be used to cleave inosine residues from a nucleic acid creating abasic sites that can be further processed by one or more enzymes such as Klenow to create blunt-ended fragments capable of blunt-ended ligation (see for example U.S. Pat. Nos. 8,673,560, 8,728,728 and 8,728,736 which are incorporated herein in their entireties).

In some embodiments, the cleaving the cleavable group produces a population of cleaved amplified nucleic acids. In some embodiments, the cleaving the cleavable group produces a plurality of cleaved amplified nucleic acids having at least one blunt end or at least one overhang end.

In some embodiments, one or more target-specific primers, target polynucleotides, target amplicons or adapters can include a cleavable moiety. Furthermore, a cleavable moiety can be located at a nucleotide position at, or near, the terminus of a target-specific primer, target polynucleotide, target amplicon or adapter. In some embodiments, a cleavable moiety can be located within 15, within 10, within 8, within 5, within 4, within 3, nucleotides of the 3′ end or the 5′ end of the nucleic acid having the cleavable moiety. In some embodiments, a cleavable moiety can be located at or near a central nucleotide in a target-specific primer. In some embodiments, one or more cleavable moieties can be present in a target-specific primer, target amplicon or adapter. In some embodiments, cleavage of one or more cleavable moiety in a target-specific primer, target amplicon or adapter can generate a plurality of nucleic acid fragments with differing melting temperatures. In one embodiment, the placement of one or more cleavable moieties in a target-specific primer, target amplicon or adapter can be regulated or manipulated by determining a melting temperature for each nucleic acid fragment, after cleavage of the cleavable moiety. In some embodiments the cleavable moiety can include a cleavable group such as uracil or uridine. In some embodiments, the cleavable group can include an inosine moiety. In some embodiments, at least 25% of the target-specific primers or target amplicons can include at least one cleavable group. In some embodiments, at least 50% of the target-specific primers or target amplicons can include at least one cleavable group. In some embodiments, at least 75% of the target-specific primers can include at least one cleavable group. In some embodiments, at least 90% of the target-specific primers can include at least one cleavable group. In some embodiments, at least 95% of the target-specific primers can include at least one cleavable group. In some embodiments, at least 98% of the target-specific primers can include at least one cleavable group. In some embodiments, each target-specific primer includes at least one cleavable group. In some embodiments, one target specific primer from a primer pair includes at least one cleavable group. In another embodiment, each target-specific primer from each primer pair can include at least one cleavable group.

In some embodiments, the disclosure relates generally to methods, compositions, systems, apparatuses and kits, comprising conducting a multiplex nucleic acid amplification reaction and further comprising ligation of an adapter. In some embodiments, at least one end of the cleaved amplified nucleic acid can be ligated to at least one adaptor to produce at least one adapter-ligated amplified nucleic acid. In some embodiments, the cleaved amplified nucleic acid can have at least one end having a substantially blunt end, which can be created by cleaving the cleavable group, optionally followed by digestion of overhangs, end-polishing or some other process whereby a blunt end is created. In some embodiment, at least one end of one or more adaptors includes a blunt end. In some embodiments, at least one end of the cleaved amplified nucleic acid can be ligated to at least one adaptor in a blunt-end ligation reaction. The cleaved amplified nucleic acid can have at least one end having an overhang end, which can be created by cleaving the cleavable group, or via restriction digestion, terminal tailing, exonuclease digestion or endonuclease digestion or via other suitable means. In some embodiment, at least one end of one or more adaptors includes a 5′ or 3′ overhang end. The length of the overhang end can include any number of nucleotides, including 1-10 or 11-20 or 21-50, or longer overhang ends. The overhang end can include any sequence, including any mixture of different bases or a homopolymer sequence (e.g., polyA, polyG, polyC or polyT). In some embodiments, at least one end of the cleaved amplified nucleic acid can be ligated to at least one adaptor in an overhang-end ligation reaction.

In some embodiments, the first end of the cleaved amplified nucleic acid can be ligated to a first adaptor, and the second end of the cleaved amplified nucleic acid can be ligated to a second adaptor, where the first and the second adaptor contain the same sequence or different sequences. Optionally, the adaptor includes an amplification primer binding site, a sequencing primer binding site, a universal sequence and/or a unique identifier sequence (e.g., barcode sequence). The amplification primer binding site on the adapter-ligated amplified nucleic acid can hybridize to an amplification primer. The sequencing primer binding site on the adapter-ligated amplified nucleic acid can hybridize to a sequencing primer. The unique identifier sequence on the adapter-ligated amplified nucleic acid can hybridize to an amplification primer or a sequencing primer.

In some embodiments, the disclosed methods (and related compositions, systems, apparatuses and kits) can include ligating at least one adapter, where the at least one adapter includes a nucleic acid sequence that is substantially non-complementary (or non-complementary) under stringent hybridizing conditions to the target polynucleotide, to the amplified target sequence, to the target amplicon and/or to any other nucleic acid molecule in the reaction mixture. In some embodiments, the at least one adapter includes a single-stranded linear oligonucleotide. In some embodiments, the at least one adapter includes a double-stranded adapter. In some embodiments, the at least one adapter includes a plurality of different single-stranded and/or double-stranded adapters in the same reaction mixture.

In some embodiments, the disclosed methods (and related compositions, systems, apparatuses and kits) can include ligating at least one adapter to at least one of the amplified target polynucleotides to produce one or more adapter-ligated amplified target polynucleotides. In some embodiments, the disclosed methods (and related compositions, systems, apparatuses and kits) can include ligating at least one adapter to at least one of the target amplicons to produce one or more adapter-ligated amplicons. In some embodiments, the ligating can include ligating an adapter to the 5′ end of the at least one amplified target polynucleotide or target amplicon. In some embodiments, the ligating can include ligating an adapter to the 3′ end of the at least one amplified target polynucleotide or target amplicon. In some embodiments, the ligating can include ligating an adapter to the 5′ end of the at least one amplified target polynucleotide or target amplicon and ligating an adapter to the 3′ end of the at least amplified target polynucleotide or target amplicon. In some embodiments, the ligating can include ligating the same adapter to the 5′ end and the 3′ end of the amplified target polynucleotide or target amplicon. In yet another embodiment, the ligating can include ligating different adapters to the 5′ end and the 3′ end of the amplified target polynucleotide or target amplicon. In some embodiments, ligation of an adapter to the 3′ end and ligation of an adapter to the 5′ end of the amplified target sequence or target amplicon can occur simultaneously. In some embodiments, ligation of an adapter at the 3′ end and ligation of an adapter at the 5′ end can occur sequentially.

In some embodiments, the methods disclosed herein (as well as related kits, systems, apparatuses and compositions) can include contacting an amplified target polynucleotide or target amplicon having a 3′ end and a 5′ end with a ligation reaction mixture. In some embodiments, a ligation reaction mixture can include one or more adapters and a ligase to produce at least one adapter-ligated amplified target polynucleotide. In some embodiments, the ligation reaction can include a DNA ligase and at least one pair of adapters, each of the pair of adapters including a different, and non-complementary, nucleic acid sequence to the other adapter in the pair of adapters. In some embodiments, none of the adapters in the ligation mixture, prior to the ligating, includes a target-specific sequence that is complementary along its length to one or more of the amplified target polynucleotides or target amplicons. In some embodiments, none of the adapters in the ligation mixture, prior to ligating, includes a sequence that is substantially complementary, or complementary, to the 3′ end or the 5′ end of an amplified target polynucleotide or target amplicon. In some embodiments, the one or more adapters are not complementary or identical to the 5′ end of the plurality of target-specific primers. In another embodiment, the one or more adapters do not include a nucleic acid sequence that is complementary or identical to the terminal 10 nucleotides at the 5′ end of the plurality of target-specific primers. Optionally, the 3′ end of an amplified target sequence or target amplicon includes about the terminal 30 nucleotides, and in some instances refers to about the terminal 15 nucleotides, or about the terminal 10 nucleotides from the 3′ end of an amplified target polynucleotide or target amplicon. In some embodiments, the 5′ end of an amplified target polynucleotide or target amplicon includes about the terminal 30 nucleotides, and in some instances refers to about the terminal 15 nucleotides, or about the terminal 10 nucleotides from the 5′ end of an amplified target polynucleotide or target amplicon. In another embodiment, the ligation reaction can include one or more adapters that further include a barcode, tag, or universal priming sequence. In yet another embodiment, the ligation reaction can include one or more adapters that are phosphorylated at the 5′ end.

In some embodiments, none of the adapters in the ligation mixture, prior to ligating, can hybridize under high stringency, to some portion of an amplified target polynucleotide or target amplicon. In some embodiments, ligating can include direct ligation of one or more adapters to one or more amplified target polynucleotide or target amplicons. In one embodiment, the ligation reaction can include a single-stranded or double-stranded adapter. In one embodiment, ligating can include performing a blunt-ended ligation. For example, the process of blunt-ended ligation can include ligating a blunt-end double-stranded amplified target polynucleotide to a blunt-ended double-stranded adapter. In one embodiment, ligating can include performing a sticky-ended ligation. For example, the process of sticky-ended ligation can include ligating a sticky-end double-stranded amplified target polynucleotide to a blunt-ended double-stranded adapter. In another embodiment, the ligating can include a single-stranded adapter. For example, the process of direct single-stranded ligation can include ligating a single-stranded amplified target polynucleotide or target amplicon to a single-stranded adapter. In this example, the ligated single-stranded adapter can be used as a template in the presence of an appropriate primer (e.g., a universal primer) to extend the appropriate primer in a template dependent manner, using the single-stranded ligation product as the template. In some embodiments, the adapter can include a double-stranded adapter that contains a partially single-stranded region, such as a single-stranded overhang. In some embodiments, the partially-single stranded region can include an “A” or “T” overhang, or a “G” or “C” overhang. In some embodiments, the ligating does not include one or more additional oligonucleotide adapters (i.e., bridging or patch oligonucleotides) prior to ligating an adapter to an amplified target polynucleotide or target amplicon.

Optionally, the disclosed methods can further include ligating one or more adapters including a universal priming sequence to the amplified product formed as a result of the target-specific primer amplification. In some embodiments, the universal priming sequence can be used in any applicable downstream process, such as universal amplification, nucleic acid enrichment, clonal amplification, bridge PCR, or nucleic acid sequencing. For example, in some embodiments, one or more adapters can be ligated to an amplified target polynucleotide. Optionally, an adapter that is ligated to an amplified target polynucleotide is susceptible to exonuclease digestion. In some embodiments, an adapter susceptible to exonuclease digestion can be ligated to the 3′ end of an amplified target polynucleotide. In some embodiments, an adapter ligated to an amplified target polynucleotide does not include a protecting group. In some embodiments, the one or more adapters do not include a protecting group that can prevent nucleic acid degradation or digestion under degrading or digesting conditions. For example, subsequent enzymatic digestion of the adapter-ligated amplified target polynucleotide in the presence of nucleic acids that do not include a protecting group, offers a means for selective digestion of the unprotected nucleic acids. In some embodiments, the one or more adapters can further include a DNA barcode or tag for any suitable method used in downstream processing.

In some embodiments, the disclosure relates generally to methods, (as well as compositions, systems, apparatuses and kits) for performing multiplex nucleic acid amplification. In some embodiments, the methods (as well as related compositions, kits, apparatuses and systems using such methods) include amplifying one or more target polynucleotides using one or more target-specific primers in the presence of polymerase under amplification conditions to produce an amplified target polynucleotide and, ligating an adapter to the amplified target polynucleotide. Further, the method can include reamplifying an adapter-ligated amplified target polynucleotide to form a reamplified adapter-ligated amplified target polynucleotide. In some embodiments, a reamplified adapter-ligated amplified target polynucleotide can be produced using no more than two rounds of target-specific selection. For example in the first round of target-specific selection, a first target-specific primer can be used under amplification conditions to produce a first amplified target polynucleotide (e.g., hybridizing the first target-specific primer to a target polynucleotide under amplification conditions and extending the hybridized first target-specific primer in a template dependent manner). While in the second round of target-specific selection, a second target-specific primer can be used that is specific for a region (e.g., the 3′ or 5′ end) of the first amplified target polynucleotide, and the second target specific primer can be used under amplification conditions to produce a second amplified target polynucleotide using no more than two rounds of target-specific amplification.

In some embodiments, the disclosure relates generally to methods, compositions, systems, apparatuses and kits for avoiding or reducing the formation of amplification artifacts (for example primer-dimers and non-specific priming) during selective amplification of one or more target polynucleotides in a population of nucleic acid molecules. In some embodiments, the disclosure relates generally to the synthesis of multiple target polynucleotides from a population of nucleic acid molecules. In some embodiments, the method comprises hybridizing one or more target-specific primer pairs to the target polynucleotide, extending a first primer of the primer pair, denaturing the extended first primer product from the population of nucleic acid molecules, hybridizing to the extended first primer product the second primer of the primer pair, extending the second primer to form a double stranded product, and digesting the target-specific primer pair away from the double stranded product to generate a plurality of amplified target polynucleotides. Optionally, the amplified target polynucleotide can be denatured to form single stranded polynucleotides prior to ligating an adapter to the amplified target polynucleotide. In some embodiments, the digesting step includes digesting one or more of the target-specific primers from the amplified target polynucleotides to create blunt-ended or sticky-end polynucleotides. In some embodiments, the double-stranded or single-stranded amplified target polynucleotides can be ligated to one or more adapters. In some embodiments, the one or more adapters can include one or more DNA barcodes or tagging sequences. In some embodiments, the amplified target polynucleotides once ligated to an adapter can undergo a nick translation reaction and/or further amplification to generate a library of adapter-ligated amplified target polynucleotides. In some embodiments, the amplified target polynucleotides can undergo a further amplification step, for example using a nucleic acid sequence within the adapter that can act as a universal priming sequence to allow further amplification of the single stranded adapter-ligated polynucleotide with an appropriate primer, thereby generating a library of adapter-ligated amplified target polynucleotides. In some embodiments, the target-specific primer pairs when hybridized to a target polynucleotide and amplified as outlined herein can generate a library of adapter-ligated amplified target polynucleotides that are from 100 to 1,000 base pairs in length, 150 to 800 base pairs in length, or 200 to 700 base pairs in length.

In some embodiments, the multiplex nucleic acid amplification reaction comprises: contacting a first plurality of target polynucleotides with a first plurality of target-specific primer pairs in a first reaction mixture, and contacting a second plurality of target polynucleotides with a second plurality of target-specific primer pairs in a second reaction mixture. In some embodiments the first and the second reaction mixtures are contained in separate reaction vessels. In some embodiments the first and the second reaction mixtures can undergo separate primer extension reactions to produce a first and a second plurality of amplicons. Optionally, the first and a second plurality of amplicons can be pooled. Optionally, the pooled plurality of amplicons can be characterized.

In some embodiments, the disclosure relates generally to methods, compositions, systems, apparatuses and kits for characterizing the plurality of amplicons, using any procedure, including: hybridizing (e.g., subtractive hybridization or microarray analysis), sequencing, detecting or determining the abundance of one or more sequences of interest. In some embodiments, the subtractive hybridization reaction can be conducted by hybridizing the plurality of amplicons with a nucleic acid having a reference sequence. In some embodiments, the microarray analysis can be conducted by hybridizing the plurality of amplicons with one or more capture probes on a microarray. Any procedure that can be used to characterize an amplicon can also be used to characterize a plurality of adapter-ligated amplified nucleic acids.

In some embodiments, the number of amplicons that contain sequences derived from a target polynucleotide can be counted and used to determine the complexity and abundance of RNA sequences of interest that are present in the sample, or can be used to calculate ratios of abundances of two different RNA sequences of interest. In some embodiments, the sequence information can be used for additional downstream analyses, including: detecting the presence of one or more RNA sequences-of-interest in the sample; detecting wild-type sequences; detecting mutant sequences; detecting gene fusion sequences; detecting splice isoforms; detecting differences in abundance levels of one or more RNA sequences compared to wild type levels; identifying mutant RNA sequences; identifying allelic variant RNA sequences; identifying single nucleotide variant RNA sequences; determining the sequence of a splice junction; determining the terminal 5′ or 3′ boundary of an RNA; or determining the abundance, or relative abundance, of an RNA; gene expression profiling; differential gene expression; or preparation of arrays by immobilizing the plurality of amplicons to a support.

In some embodiments, the detecting comprises hybridizing a nucleic acid probe with the plurality of amplicons. In some embodiments, the detecting further comprises detecting the presence of a complex formed by hybridization of the nucleic acid probe with at least one amplicon. Optionally, the nucleic acid probe includes a detectable moiety.

In some embodiments, the detecting comprises re-amplifying the plurality of amplicons. Optionally, the re-amplifying comprises hybridizing an amplification primer to the amplification primer binding site on the amplicon or the adapter-ligated amplified nucleic acid, and conducting a nucleic acid amplification reaction.

In some embodiment, the number of amplicons or adaptor-ligated amplified nucleic acids that contain sequences derived from a target polynucleotide can be quantified by hybridization in a microarray analysis, or by sequencing. Optionally, the sequences of the different amplicons (e.g., a sequencing read) can be aligned against one or more reference sequences. Optionally, the number of a sequencing read from a particular amplicon that aligns to a particular reference sequence can be used to generate a read count of that amplicon sequence. In the same manner, a read count of different amplicon sequences can be generated. Optionally, the read counts of two or more amplicon sequences can be converted to relative abundance, or can be used to calculate a ratio of two different amplicon sequences within the same reaction mixtures, or within different reaction mixtures.

In some embodiments, the sequencing data is counted and tallied. In some embodiments, a plurality of amplicons or adaptor-ligated amplified nucleic acids is generated according to the present teachings. At least some, or all, of the plurality of amplicons or adaptor-ligated amplified nucleic acids are sequenced to generate a plurality of sequence reads. In some embodiments, each sequence read represents a target polynucleotide sequence (or a portion thereof) which is contained in the amplicons or adaptor-ligated amplified nucleic acids. In some embodiments, some or all of the plurality of amplicons or adaptor-ligated amplified nucleic acids are sequenced. The sequence reads are compared and/or aligned with sequences of interest in a reference list. In some embodiments, the sequence reads representing different target polynucleotides are counted using a software program, for example using an RNA plugin from Torrent Suite (Torrent Suite™ Software, version 4.0.2, user interface guide, document revision November 2013 Rev. A).

Optionally, the sequence reads can be aligned to one or more reference sequences and compared to a reference list to determine a read count for one or more sequences of interest. Optionally, a sequence variant includes a sequencing read that differs from one or more reference sequences. Optionally, the sequencing reads that differ from the reference sequence (e.g., a sequence variant) are identified. Optionally, reads that align to the reference sequences that do not correspond to the one or more sequences of interest can be retained or discarded.

Optionally, methods and systems described in U.S. published application Nos. 2013/0073214 and 2013/0268207 (herein incorporated by reference in their entireties) can be used to identify sequencing read variants. In some embodiments, the reference list contains nucleic acid sequences of interest that are associated with a healthy cell, or any disease or cancer. In some embodiments, reconstruction of a longer target sequence can be achieved by assembling two or more sequence reads. Sequence assembly includes alignment of two or more sequence reads against a reference sequence of interest, or alignment of overlapping sequences in two or more sequence reads. In some embodiments, the need for sequence assembly is substantially reduced or obviated, because a single pair of target-specific primers is configured to generate a single sequence for each target polynucleotide. In some embodiments, when a single primer pair is used to generate a single sequence for each target polynucleotide, a sequence read assembly is not performed. In some embodiments, the number of a sequence read that aligns with a particular sequence of interest is counted. For example, the number of a first sequence read that aligns with a first sequence of interest is counted, and the number of a second sequence read that aligns with a second sequence of interest is counted. Optionally, at least some or all of the sequence reads are counted. The count of the number of first sequence reads is tallied, and the count of the number of second sequence reads is also tallied. The total number of tallied first and second reads are compared to each other, and the comparison can be expressed as a ratio or percentage. Optionally, the relative abundance of a first transcript and a second transcript can be obtained by comparing the total number of tallied first and second reads. Optionally, the sequence reads have perfect or imperfect alignment with their respective sequence of interest in the reference lists. Optionally, the sequence reads have one or more mutations that result in imperfect alignment with the reference sequence of interest. For example, at least one sequence read includes mutations comprising one or more deletions, insertions, or substitutions of one or more nucleotides, inversions, rearrangements, fusions, truncations, and/or variant or abnormal splice junction sequences.

In some embodiments, sequence coverage includes the number of reads that map to a location of a reference genome. In some embodiments, sequence coverage is used to calculate percentage of an allele (e.g., an allelic variant or a mutant allele). For example, calculating a percentage of an allele includes the count of an allele divided by the coverage at that locus in the reference genome.

In some embodiments, the percentage of a first and a second allele can be compared and expressed as a ratio or percentage. In some embodiments, the percentage of a normal allele and a cancer allele can be compared.

Optionally, the percentage of the cancer allele decreases while the percentage of the normal allele increases. Optionally, the percentage of the cancer allele increases while the percentage of the normal allele decreases.

In some embodiments, the frequency of at least one sequence variant can be determined. For example, low frequency sequence variants includes sequence variants that occur in fewer than about 60%, or about 50%, or about 40%, or lower percent occurrence of the sequencing reads.

In some embodiments, the first and second sequencing reads may correspond to different amplicon sequences generated by common primer or pair of primers. For example, the first and second reads may be distinguished by a single nucleotide polymorphism, an indel, the presence or absence of a gene fusion, or the like. In some embodiments where the first and second reads result from one or more common primers, relative abundance of the first read can be calculated by comparing the read count for the first read with the total count of reads associated with the common primer. In some embodiments, the relative abundance of a gene fusion amplicon can be determined by comparing to the read count for the gene fusion amplicon to the total number of reads for amplicons associated with the first primer from the first gene and the second primer from the second gene. In some embodiments, the read count for an amplicon with a large deletion or gene fusion can be compared to the average of read counts for reads associated with the first primer and reads associated with the second primer.

In some embodiments, the relative abundance of the same transcript within samples taken before and after treatment of interest (e.g., exposure of the samples to drugs, stimuli, feeding, immune challenge, etc) can be compared and determined. In some embodiments, relative abundances of different transcripts can be compared.

In some embodiments, the detecting comprises sequencing the plurality of amplicons. In some embodiments, the identity of the sequences of the plurality of amplicons can be determined. Optionally, the sequencing procedure comprises hybridizing a sequencing primer to the sequencing primer binding site on the amplicon or the adapter-ligated amplified nucleic acid, and conducting a sequencing reaction. Optionally, the sequencing comprises a massively parallel sequencing procedure, or a gel electrophoresis procedure.

In some embodiments, the detecting comprises determining the abundance of an RNA sequence of interest, by quantifying the number of the amplicons containing sequences derived from RNA or derived from the plurality of target polynucleotides. In some embodiments, the abundance of a first RNA sequence of interest can be determined by quantifying the number of the amplicons containing sequences derived from a first RNA or derived from a first target polynucleotide. In some embodiments, the abundance of a second RNA sequence of interest can be determined by quantifying the number of the amplicons containing sequences derived from a second RNA or derived from a second target polynucleotide. Optionally, the quantifying includes counting the number of amplicons containing sequences derived from RNA or derived from the plurality of target polynucleotides. In some embodiments, the detecting further comprises comparing the abundance of the amplicons containing sequences derived from a first RNA or derived from a first target polynucleotide, with the abundance of the amplicons containing sequences derived from a second RNA or derived from a second target polynucleotide.

In some embodiments, the detecting comprises determining differences in abundance levels of one or more RNA sequences compared to wild type or normal levels, by quantifying the number of the amplicons containing sequences derived from RNA or derived from the plurality of target polynucleotides. In some embodiments, normal levels of an RNA sequence of interest can be determined by quantifying the number of the amplicons containing sequences derived from the RNA from a first sample (e.g., a sample of normal cells). In some embodiments, different levels of the same RNA sequence of interest can be determined by quantifying the number of the amplicons containing sequences derived from the RNA from a second sample (e.g., a sample of abnormal or diseased cells). In some embodiments, the detecting further comprises comparing the levels (amounts) of the amplicons containing sequences derived from RNA from a sample of normal cells, with levels (amounts) of the amplicons containing sequences derived from RNA from a sample of abnormal or diseased cells. In some embodiments, the abnormal cells include diseased cells, tumor cells, cells challenged with nutrient starvation, or cells challenged with a chemical compound or physical stress. In some embodiments, determining the difference in abundance levels of one or more RNA sequences in a sample is used to detect changes in the expression level of a gene in a first cell (or in a first plurality of cells) compared to the expression level of the same gene in a second cell (or in a second plurality of cells). In some embodiments, the expression level of the gene increases or decreases.

In some embodiments, the detecting comprises determining a ratio of the number of amplicons containing a sequence derived from a first RNA, and the number of amplicons containing a sequence derived from a second RNA. Optionally, determining the ratio includes counting the number of amplicons containing a sequence derived from the first RNA and the number of amplicons containing a sequence derived from the second RNA.

In some embodiments, the detecting comprises determining a ratio of the number of amplicons containing a sequence derived from a first target polynucleotide, and the number of amplicons containing a sequence derived from a second target polynucleotide. Optionally, determining the ratio includes counting the number of amplicons containing a sequence derived from the first target polynucleotide and the number of amplicons containing a sequence derived from the second target polynucleotide.

In some embodiments, the number of amplicons or adaptor-ligated amplified nucleic acids that contain sequences derived from a target polynucleotide can be counted and tallied, and used to determine the amount of amount of one or more RNA transcripts of interest that are present in any cell or tissue. For example, the cell or tissue is subjected to an extraction procedure to produce an RNA sample. Some or all of the RNA in the sample is converted to a plurality of cDNA using any suitable procedure. In some embodiments, the plurality of cDNA can be generated by conducting a reverse transcription reaction, comprising: contacting some or all of the RNA with primers, at least one enzyme having RNA-dependent DNA polymerase activity, and a plurality of nucleotides, under conditions suitable for reverse transcription. Optionally, the primers can be random-sequence primers, polyT primers, or target-specific primers. Optionally, the RNA-dependent DNA polymerase enzyme can be a reverse transcriptase. In some embodiments, the plurality of cDNA can be generated by ligating some or all of the RNA to double-stranded adaptors to produce ligation products having single-stranded RNA joined, at one end or at both ends, to one strand of a double-stranded adaptor. Optionally, the double-stranded adaptors comprise RNA/DNA or DNA/DNA. The ligation products can be heated to remove one of the strands of the adaptors, thereby generating a single-stranded RNA template. In some embodiments, some or all of the single-stranded RNA templates are converted to a plurality of cDNA using a reverse transcription procedure.

In some embodiments, the plurality of cDNA contains different sequences derived from different RNA in the sample. For example, the plurality of cDNA contains at least a first cDNA derived from a first RNA transcript in the sample, and a second cDNA derived from a second RNA transcript in the sample. In some embodiments, the sequence complexity and amounts of different cDNAs reflects the sequence complexity and amounts of different RNA sequences found in the RNA sample from which the cDNA was derived. For example, the amount of the first cDNA relative to the amount of the second cDNA is similar to the relative amounts of the first and second RNA transcripts in the sample.

In some embodiments, the plurality of cDNA is contacted with a plurality of target-specific primer pairs, under conditions suitable to hybridize at least one of the target-specific primer pairs to at least one cDNA to form at least one nucleic acid duplex. In some embodiments, each of the plurality of target-specific primer pairs hybridizes to a different target cDNA sequence. In some embodiments, a single pair of target-specific primers will hybridize to any give target cDNA sequence. In some embodiments, at least one cDNA molecule that is generated from the RNA sample contains a target sequence. For example, a single pair of target-specific primers will hybridize to a cDNA sequence and mediate amplification to produce amplicons that represent a transcript of interest. In some embodiments, the plurality of cDNA may, or may not, contain all target cDNA sequences. In some embodiments, a primer extension reaction is conducted on the nucleic acid duplexes, in a template-dependent fashion, to form a plurality of amplicons. In some embodiments, each amplicon contains a sequence derived from an RNA in the sample.

In some embodiments, the plurality of amplicons contains an amount of different sequences derived from that reflects the sequence complexity and relative amounts of different polynucleotide sequences found in the sample from which the plurality of amplicons was derived. For example, the amount of the first amplicon relative to the amount of the second amplicon is similar to the relative amounts of the first and second polynucleotides in the sample.

In some embodiments, the plurality of amplicons contains different sequences derived from different sources within a mixed sample. For example, the plurality of amplicons can contain at least a first amplicon derived from a normal cell in the sample, and the plurality of amplicons contains a second amplicon derived from a tumor cell in the sample. Alternatively, the plurality of amplicons can contain at least a first amplicon derived from maternal polynucleotide in the sample, and the plurality of amplicons can contain a second amplicon derived from a fetal polynucleotide in the sample. In some embodiments, the plurality of amplicons can contain at least a first amplicon derived from a first chromosome, and the plurality of amplicons can contain a second amplicon derived from a second chromosome in the sample. In some embodiments, the number of amplicons containing sequence corresponding to, or derived from, the first and second amplicons (or first and second template polynucleotides) is counted and tallied for each of the first and second amplicons. For example, the number of amplicons containing a first target sequence of interest can be counted and tallied to obtain a first number. In some embodiments, the number of amplicons containing a second target sequence of interest are counted and tallied to obtain a second number. Optionally, the resulting counts, tallies and/or numbers (e.g., first and second numbers) can be used to estimate the relative abundance of the first and second target sequences of interest within the sample. For example, the resulting counts, tallies or numbers (e.g., first and second numbers) can be used to determine the presence of a chromosomal aneuploidy, or a copy number change, or the percentage of polynucleotides including a variant or substitution at a given position. In some embodiments, the resulting counts, tallies or numbers (e.g., first and second numbers) can be used to determine the proportion of minor DNA sequences present amongst a majority. For example, the resulting counts, tallies or numbers (e.g., first and second numbers) can be used to determine the proportion of fetal DNA present amongst a background of maternal DNA, or the proportion of tumor DNA (i.e., DNA derived from a tumor cell, which may be extracted from the cell or present within the plasma) present amongst a background of normal DNA (i.e., non-tumor DNA).

In some embodiments, the plurality of amplicons contains different sequences derived from different RNA in the sample. For example, the plurality of amplicons contains at least a first amplicon derived from a first RNA transcript in the sample, and the plurality of amplicons contains a second amplicon derived from a second RNA transcript in the sample. In some embodiments, the plurality of amplicons contains an amount of different sequences that reflects the sequence complexity and amounts of different RNA sequences found in the RNA sample from which the plurality of amplicons was derived. For example, the amount of the first amplicon relative to the amount of the second amplicon is similar to the relative amounts of the first and second RNAs in the sample.

Optionally, the amplicons are ligated to nucleic acid adaptors to generate adaptor-ligated amplified nucleic acids. In some embodiments, the amplicons (or the adaptor-ligated amplified nucleic acids) are characterized, for example, by sequencing to generate sequencing data.

Optionally, the amplicons (or the adaptor-ligated amplified nucleic acids) can be characterized by massively parallel sequencing or sequencing using gel electrophoresis. In some embodiments, different target sequences are identified from the sequencing data. The number of different target sequences is counted and tallied, to generate information pertaining the different transcript sequences, and abundances of the transcripts, contained in the initial RNA sample. For example, the number of a first amplicon derived from a first RNA transcript are counted and tallied. In a similar manner, the number of a second amplicon derived from a second RNA transcript are counted and tallied. The number of first and second amplicons can be expressed as a percentage or a ratio relative to each other. One skilled in the art will readily recognize that more than two transcripts of interest can be analyzed using any of the methods described herein.

In some embodiments, the counted and tallied sequencing information is used to determine gene expression of one or more transcripts of interest contained in a single RNA sample or in two or more RNA samples. In some embodiments, gene expression includes transcription of at least one DNA sequence of interest in one or more cells. The RNA transcripts present in a cell, at a given time, represent steady-state RNA levels resulting from transcription of DNA sequences in the cells, and post-transcriptional modification and/or degradation of the RNA. At least some of the RNA transcripts in the cells may be post-transcriptionally modified (including splicing) and/or degraded (e.g., RNA turnover). Thus, transcription, post-transcriptional modification and degradation will results in different RNA transcripts present in the cells at different abundances. The types of RNA sequences, and their abundances, can change with onset of cell cycle progression, cell differentiation, cell development, abnormality or a disease, or can change in response to stimuli with a physical or chemical challenge. The types of RNA transcripts and their abundances may differ in different types of cells (e.g., pancreas vs. ovary cells). The RNA present in the cells may include coding and/or non-coding transcripts. In some embodiments, the sequencing information is used to determine the presence or absence of one or more RNA transcripts of interest in the one or more samples.

In some embodiments, the counted and tallied sequencing information is used to measure the abundance of transcripts of interest contained in a single RNA sample, by comparing the amount of a first amplicon of interest with the amount of a second amplicon of interest, where the first and second amplicons of interest are derived from first and second RNA transcripts (respectively) present in the same RNA sample. It will be appreciated by the skilled artisan that the amount of more than two different amplicons present in the same sample can be compared. Analysis of the counted and tallied sequencing information may show that expression levels of the first and second transcripts of interest in the sample is the same or is different. The difference in the amounts of the first and second transcripts of interest can be mathematically expressed as a -fold change or percent change.

In some embodiments, the counted and tallied sequencing information is used to measure the abundance of transcripts of interest contained in a reference RNA sample and contained in one or more test samples, by comparing the amount of a first amplicon of interest from the reference sample with the amount of a second amplicon of interest from the test sample, where the first amplicons of interest are derived from a first RNA transcript present in the reference sample and the second amplicons of interest are derived from a second RNA transcript present in the test sample. The first and second RNA transcripts can be the same or different transcripts of interest. It will be appreciated by the skilled artisan that the amount of two or more different amplicons from the reference and the test samples can be compared. Analysis of the counted and tallied sequencing information may show that expression levels of the transcripts of interest in the reference and test samples changes, or remains unchanged. The changes in the transcripts of interest in the reference and test samples can be mathematically expressed as a -fold change or percent change.

The changes in abundance of the transcripts of interest may correlate with a change within the cells, or correlate with an abnormal or diseased cell, or correlate with a physical- or chemical-induced challenge. The test samples can be derived from cells suspected of containing different types and/or different abundances of at least one transcript of interest.

In some embodiments, the counted and tallied sequencing information is used to determine copy number changes of one or more transcripts of interest contained in a single RNA sample or in two or more RNA samples. In some embodiments, changes in copy number of a transcript in a cell can arise from transcription of a DNA sequence in a cell, where the DNA sequence has an increase or decrease in copy number (e.g., aneuploidy). For example, a cell may contain a trisomic chromosome arm resulting in three copies of a DNA sequence of interest, or the cell may contain a missing chromosome arm resulting in one copy of the DNA sequence of interest. In another example, a diploid cell may contain one extra copy of a DNA sequence of interest on a chromosome arm resulting in three copies of a DNA sequence of interest, or the cell may contain a deletion of a DNA sequence of interest resulting in one copy of the DNA sequence of interest. In a cell containing an abnormal copy number of the DNA sequence of interest, transcription of the DNA sequence of interest may result in an abnormal copy number of the RNA transcript of interest. In some embodiments, in a normal diploid cell, the DNA sequence of interest on both paired chromosomes is transcribed. In some embodiments, the amount of steady-state RNA transcript of interest produced from the paired chromosomes is approximately equal.

In some embodiments, the counted and tallied sequencing information is used to measure the copy number of transcripts of interest contained in a reference RNA sample and contained in one or more test samples, by comparing the amount of a first amplicon of interest from the reference sample with the amount of a second amplicon of interest from the test sample, where the first amplicons of interest are derived from a first RNA transcript present in the reference sample and the second amplicons of interest are derived from a second RNA transcript present in the test sample. In some embodiments, the first and second RNA transcripts of interest can have the same or different sequences. It will be appreciated by the skilled artisan that the amount of two or more different amplicons from the reference and the test samples can be compared. Analysis of the counted and tallied sequencing information may show that the copy number of the transcripts of interest in the reference and test samples changes, or remains unchanged. The changes in the transcripts of interest can be mathematically expressed as a -fold change or percent change.

For example, the counted and tallied sequencing information may yield N copy number of the transcript of interest from the reference sample, and 1.5×N (1.5 times N) copy number of the transcript of interest from the test sample. This data indicates that the test sample contains one extra copy number of the transcript of interest, which correlates with a test sample containing three copies of the transcript of interest.

In another example, the counted and tallied sequencing information may yield N copy number of the transcript of interest from the reference sample, and a 0.5×N (0.5 times N) copy number of the transcript of interest from the test sample. This data indicates that the test sample is missing one copy of the transcript of interest, which correlates with a test sample containing one copy of the transcript of interest.

The changes in copy number of the transcripts of interest may correlate with a change within the cells, or correlate with an abnormal or diseased cell, or correlate with a physical- or chemical-induced challenge. The test samples can be derived from cells suspected of containing different types and/or different copy numbers of at least one transcript of interest.

In some embodiments, the counted and tallied sequencing information is used to detect gene fusion RNA transcripts contained in a single RNA sample or in two or more RNA samples. In some embodiments, a gene fusion RNA transcript includes a chimeric RNA transcript having two or more sequences joined together that are not normally found joined together in a transcript. The gene fusion RNA transcripts can arise from transcription of DNA gene fusion sequences in the cell. In some embodiments, the DNA and RNA gene fusion sequences need not include entire genes, or entire exons or introns of genes.

In some embodiments, the DNA gene fusion sequences can contain a promoter from a first gene joined to the coding region of a second gene. Optionally, the promoter causes altered transcription (e.g., increase or decrease) of the co-joined coding region in the cell. Optionally, the RNA gene fusion transcripts contain a junction sequence containing at least part of the promoter sequence or a sequence of the first gene joined to the second gene sequence.

In some embodiments, the DNA and RNA gene fusion sequences contain at least one exon or intron region of a first gene joined to a second gene sequence, which may lead to altered RNA splicing events. Optionally, the RNA gene fusion sequence contains abnormal spliced or unspliced sequences.

In some embodiments, the DNA and RNA gene fusion sequences contain a first gene sequence joined to a second gene sequence. Optionally, the RNA gene fusion transcript undergoes altered folding into a secondary structure that causes an abnormality in the cell.

In some embodiments, the DNA and RNA gene fusion sequences contain a first gene sequence joined to a second gene sequence. Optionally, the RNA gene fusion transcript undergoes altered degradation rates that causes an abnormality in the cell.

In some embodiments, the presence of abnormal RNA gene fusion sequences or abnormal amounts of an RNA gene fusion transcripts in the cell, may cause cellular abnormality such as abnormal cell growth or function, or tumor formation, or can lead to diseased tissue development, or can lead to cell death.

In some embodiments, the sequencing information is used to determine the presence or absence of one or more RNA gene fusion transcripts in the one or more RNA samples.

In some embodiments, the counted and tallied sequencing information is used to measure the abundance of RNA gene fusion transcripts contained in a single RNA sample, by comparing the amount of a reference amplicon of interest (e.g., having a normal sequence) with the amount of a second amplicon having an RNA gene fusion sequence. In some embodiments, the reference amplicons and gene fusion amplicons are derived from a reference and second RNA transcript (respectively) present in the same RNA sample. It will be appreciated by the skilled artisan that the amount of more than two different amplicons can be compared. Analysis of the counted and tallied sequencing information may show that expression levels of the reference and gene fusion transcripts of interest in the sample is the same or is different. The difference in the amounts of the reference and gene fusion transcripts of interest can be mathematically expressed as a -fold change or percent change.

In some embodiments, the counted and tallied sequencing information is used to measure the abundance of a reference transcript contained in a reference RNA sample and the abundance of RNA gene fusion transcripts contained in one or more test samples, by comparing the amount of a reference amplicon of interest (e.g., a normal transcript) from the reference sample with the amount of a second amplicon having a fusion sequence from the test sample. In some embodiments, the reference amplicons are derived from a first RNA transcript present in the reference sample and the second amplicons are derived from one or more RNA fusion transcripts present in the test sample. It will be appreciated by the skilled artisan that the amount of two or more different amplicons from the reference and the test samples can be compared. Analysis of the counted and tallied sequencing information may show that expression levels of the transcripts of interest in the reference and test samples changes, or remains unchanged. The changes in the transcripts of interest can be mathematically expressed as a -fold change or percent change.

The presence and abundance of fusion gene transcripts may correlate with a change within the cells, or may correlate with an abnormal or diseased cell. The test samples can be derived from cells suspected of containing gene fusion transcripts, including normal, abnormal or diseased cells.

In some embodiments, the disclosure relates generally to method, and related compositions, systems, kits and apparatuses, for attaching one or more amplicons to a support. In some embodiments, any procedure that can be used to attach an amplicon to a support can also be used to attach one or more adapter-ligated amplified nucleic acid to a support.

In some embodiments, the amplicon can be modified for attachment to a support. For example, the amplicon can be amino-modified for attachment to a support (e.g., particles or a planar support). In some embodiments, an amino-modified nucleic acid fragment can be attached to a support that is coated with a carboxylic acid. In some embodiments, an amino-modified nucleic acid can be reacted with EDC (or EDAC) for attachment to a carboxylic acid coated support (with or without NHS). In some embodiments, the amplicon can be attached to particles, such as Ion Sphere™ particles (Life Technologies).

In some embodiments, a support can include an outer or top-most layer or boundary of an object. In some embodiments, a support includes a solid surface or semi-solid surface. In some embodiments, a support can be porous or non-porous. In some embodiments, a support can be a planar surface, as well as concave, convex, or any combination thereof. In some embodiments, a support can be a bead, particle, sphere, filter, flowcell, or gel. In some embodiments, a support includes the inner walls of a capillary, a channel, a well, groove, channel, reservoir. In some embodiments, a support can have texture (e.g., etched, cavitated, pores, three-dimensional scaffolds or bumps). In some embodiments, a support can be made from materials such as glass, borosilicate glass, silica, quartz, fused quartz, mica, polyacrylamide, plastic polystyrene, polycarbonate, polymethacrylate (PMA), polymethyl methacrylate (PMMA), polydimethylsiloxane (PDMS), silicon, germanium, graphite, ceramics, silicon, semiconductor, high refractive index dielectrics, crystals, gels, polymers, or films (e.g., films of gold, silver, aluminum, or diamond). In some embodiments, the amplicons can be arranged on a support in a random pattern, organized pattern, rectilinear pattern, hexagonal pattern, or addressable array pattern.

In some embodiments, the amplicons can be modified to attach to one member of a binding partner (e.g., biotin). In some embodiments, a biotinylated nucleic acid fragment can be attached to another member of a binding partner (e.g., avidin-like, such as streptavidin) which is attached to a support.

In some embodiments, molecules that function as binding partners include: biotin (and its derivatives) and their binding partners avidin, streptavidin (and their derivatives); His-tags which bind with nickel, cobalt or copper; cysteine, histidine, or histidine patch which bind Ni-NTA; maltose which binds with maltose binding protein (MBP); lectin-carbohydrate binding partners; calcium-calcium binding protein (CBP); acetylcholine and receptor-acetylcholine; protein A and binding partner anti-FLAG antibody; GST and binding partner glutathione; uracil DNA glycosylase (UDG) and ugi (uracil-DNA glycosylase inhibitor) protein; antigen or epitope tags which bind to antibody or antibody fragments, particularly antigens such as digoxigenin, fluorescein, dinitrophenol or bromodeoxyuridine and their respective antibodies; mouse immunoglobulin and goat anti-mouse immunoglobulin; IgG bound and protein A; receptor-receptor agonist or receptor antagonist; enzyme-enzyme cofactors; enzyme-enzyme inhibitors; and thyroxine-cortisol. Another binding partner for biotin can be a biotin-binding protein from chicken (Hytonen, et al., BMC Structural Biology 7:8).

In some embodiments, the disclosure relates generally to method, and related compositions, systems, kits and apparatuses, comprise sequencing any of the amplified target nucleic acids (e.g., amplicons or adapter-ligated amplified nucleic acids) generated according to the present teachings. In some embodiments, any type of sequencing platform can be employed, including: size-separation via gel electrophoresis, sequencing by oligonucleotide probe ligation and detection (e.g., SOLiD™ from Life Technologies, WO 2006/084131), probe-anchor ligation sequencing (e.g., Complete Genomics™ or Polonator™), sequencing-by-synthesis (e.g., Genetic Analyzer and HiSeg™, from Illumina), pyrophosphate sequencing (e.g., Genome Sequencer FLX from 454 Life Sciences), ion-sensitive sequencing (e.g., Personal Genome Machine (PGM™) and Ion Proton™ Sequencer, both from Ion Torrent Systems, Inc.), and single molecule sequencing platforms (e.g., HeliScope™ from Helicos™).

In one embodiment, a multiplex nucleic acid amplification method is disclosed herein that includes (a) amplifying one or more target polynucleotides using one or more target-specific primers in the presence of polymerase to produce an amplified target polynucleotide, and (b) ligating an adapter to the amplified target polynucleotide to form an adapter-ligated amplified target polynucleotide. In some embodiments, amplifying can be performed in solution such that an amplified target polynucleotide or a target-specific primer is not linked to a solid support or surface. In some embodiments, ligating can be performed in solution such that an amplified target polynucleotide or an adapter is not linked to a solid support or surface. In another embodiment, amplifying and ligating can be performed in solution such that an amplified target polynucleotide, a target-specific primer or an adapter is not linked to a solid support or surface. In yet another embodiment, the amplifying can be performed on a solid support or surface, such as a flow cell, an array, a nucleic acid sequencing bead, and the like. In another embodiment, the ligating can be performed on an amplified target polynucleotide that is attached to a solid support or surface, such as a flow cell, an array, a nucleic acid sequencing bead, and the like. In some embodiments, one of more of the plurality of target polynucleotides amplified using one or more of the disclosed methods can be used in DNA sequencing, such as any applicable next-generation sequencing platform. A variety of next-generation sequencing platforms are available that can use of one or more of the products from the amplification and synthesis methods disclosed herein. For example, next generation sequencing platforms made by Life Technologies (CA)(e.g., Ion Torrent's PGM and Proton platforms), Illumina (CA)(e.g., MiSeq, HiSeq, and X-10 platforms), Roche, Helicos, and Pacific Biosciences sequencing platforms are capable of utilizing the methods (as well as compositions, systems, apparatuses and kits) as disclosed herein for nucleic acid sequencing and/or nucleic acid analysis. In some embodiments, one or more of the plurality of target polynucleotides amplified or synthesized using one or more of the methods disclosed herein can be used to determine (or estimate) the level of mRNA expression of one or more active genes in a RNA transcriptome. In some embodiments, the level of mRNA expression of one or more active genes in a RNA transcriptome may be determined as an over-expression or under-expression of mRNA as compared to a known, matched, or reference sample. In some embodiments, the over-expression of one or more active genes in the RNA transcriptome can be indicative of a diseased or abnormal state. In some embodiments, the under-expression of one or more active genes in the RNA transcriptome can be indicative of a diseased or abnormal state.

In some embodiments, any amplified target nucleic acids (e.g., amplicons or adapter-ligated amplified target nucleic acids) that has been generated according to the present teachings, can be attached to a solid support. For example, a bridge amplification reaction can be conducted to attach the adapter-ligated amplified target nucleic acids to a planar support (e.g., flowcell) or beads. Individual amplified target nucleic acids are ligated to a first universal adaptor at one end and a second universal adaptor at the other end to generate a population of adapter-ligated amplified target nucleic acids. In some embodiments, the first and second adaptors have different sequences. In some embodiments, the first and/or second adaptor includes a universal sequencing primer sequence. In some embodiments, at least two of the amplified target nucleic acids have different sequences. The population of adapter-ligated amplified target nucleic acids is rendered single-stranded. At least a portion of the population of single-stranded adapter-ligated amplified target nucleic acids is hybridized to capture primers that are attached to a support. The support can include a plurality of first and second capture primer having different sequences. In the hybridization step, the first universal adaptor hybridizes with the first capture primer, and a primer extension reaction extends the first capture primer to generate a first capture primer extension product having a complementary sequence of the second adaptor at one end. The primer extension reaction employs the adapter-ligated amplified target nucleic acid as a template. The template molecule is removed. The first capture primer extension product bends (e.g., arches) so that the second adaptor complementary sequence can hybridize to a nearby second capture primer, and a primer extension reaction extends the second capture primer to generate a second capture primer extension product having a complementary sequence of the first adaptor at one end, and forming a double-stranded bridge molecule. The double-stranded bridge is denatured to yield two single-stranded, immobilized target nucleic acids. One of the single-stranded, immobilized target nucleic acids has a first primer (or complementary sequence thereof) which is attached to the support and the other end of the molecule has a second primer sequence (or complementary sequence thereof) that can hybridize to a nearby second capture primer to start another bridge amplification reaction. The other single-stranded, immobilized target nucleic acids has a second primer (or complementary sequence thereof) which is attached to the support and the other end of the molecule has a first primer sequence (or complementary sequence thereof) that can hybridize to a nearby first capture primer to start another bridge amplification reaction. Repeat cycles of bridge amplification produce a plurality of amplified target nucleic acids that are attached to the support. The cycles of bridge amplification can be conducted under isothermal conditions. Examples of compositions and methods for bridge amplification are found in U.S. Pat. Nos. 7,790,418, 7,985,565, 8,143,008 and 8,895,249.

In some embodiments, any amplified target nucleic acids (e.g., amplicons or adapter-ligated amplified target nucleic acids) that has been generated according to the present teachings, can be attached to a solid support. For example, a template walking reaction can be conducted to attach the adapter-ligated amplified target nucleic acids to a planar support (e.g., flowcell) or beads. Individual amplified target nucleic acids are ligated to a first universal adaptor at one end and a second universal adaptor at the other end to generate a population of adapter-ligated amplified target nucleic acids. In some embodiments, the first and second adaptors have different sequences. In some embodiments, the first and/or second adaptor includes a universal sequencing primer sequence. In some embodiments, the first and second adaptors have different sequences. In some embodiments, the first adaptor includes a universal amplification primer sequence that differs from the universal amplification sequence in the second adaptor. In some embodiments, at least two of the amplified target nucleic acids have different sequences. The population of adapter-ligated amplified target nucleic acids is rendered single-stranded. At least a portion of the population of single-stranded adapter-ligated amplified target nucleic acids is hybridized to capture primers that are attached to a support. The support can include a plurality of immobilized capture primers, where the 3′ end of the capture primers includes the same sequence. In some embodiments, the 3′ end of the capture primers includes a sequence having a low Tₘ (melting temperature) sequence. In the hybridization step, the first universal adaptor hybridizes with a first immobilized capture primer, and a primer extension reaction extends the first capture primer to generate a first capture primer extension product having a complementary sequence of the second adaptor at one end. The primer extension reaction employs the adapter-ligated amplified target nucleic acid as a template. The template molecule (which is hybridized along its length to the extension product) undergoes localized denaturation at the first adaptor region that contains the low Tₘ region, and the first adaptor region rehybridizes to a nearby capture primer (second capture primer), while the remainder of the template molecule is hybridized to the extension product. Primer extension of the second capture primer, serves to denature the portion of the template molecule that is still hybridized with the first extension product, and generates a second capture primer extension product. Repeat cycles of template walking include hybridizing the first adaptor region to a nearby capture primer, primer extension, localized denaturation, re-hybridization with a different nearby capture primer, and primer extension, produces a plurality of amplified target nucleic acids that are attached to the support. The cycles of template walking can be conducted under isothermal conditions. Examples of compositions and methods for nucleic acid template walking are found in U.S. published application Nos. 2012/0156728 and 2013/0203607.

In some embodiments, any amplified target nucleic acids (e.g., amplicons or adapter-ligated amplified target nucleic acids) that has been generated according to the present teachings, can be attached to a solid support. For example, a recombinase-polymerase amplification (RPA) reaction can be conducted under aqueous conditions to attach the adapter-ligated amplified target nucleic acids to a planar support (e.g., flowcell) or to beads. Individual amplified target nucleic acids are ligated to a first universal adaptor at one end and a second universal adaptor at the other end to generate a population of adapter-ligated amplified target nucleic acids. In some embodiments, the first and second adaptors have different sequences. In some embodiments, the first and/or second adaptor includes a universal sequencing primer sequence. In some embodiments, the first and second adaptors have different sequences. In some embodiments, the first adaptor includes a universal amplification primer sequence that differs from the universal amplification sequence in the second adaptor. In some embodiments, at least two of the amplified target nucleic acids have different sequences. The population of adapter-ligated amplified target nucleic acids is rendered single-stranded. In a single reaction mixture, the single-stranded nucleic acids are reacted with: (i) a plurality of beads having a plurality of capture primers attached thereon; (ii) a plurality of soluble reverse primers; (iii) polymerase; and (iv) a plurality of nucleotides. In some embodiments, the single reaction mixture also includes a forward fusion primer serves as a splint molecule that can hybridize to a capture primer and the first adaptor sequence which is joined to the target nucleic acid. In embodiments using the forward fusion primer, the first adaptor sequence which is joined to the target nucleic acid can hybridize with a portion of the fusion primer, but the first adaptor lacks a sequence that can hybridize to the capture primer on the bead. In some embodiments, the single reaction mixture further includes a recombinase (e.g., T4 uvsX), and optionally accessory proteins, including recombinase loading factor (e.g., T4 uvsY) and/or single-stranded binding protein (T4 gp32). The single reaction mixture can be incubated under conditions suitable for conducting nucleic acid amplification. In some embodiments, the fusion primer hybridizes to the first adaptor sequence, and a primer extension reaction yields a fusion primer extension product which includes a sequence that can hybridize to the capture primer on the bead. The soluble reverse primer hybridizes with the fusion primer extension product, and a primer extension reaction yields a reverse primer extension product. The reverse primer extension product can hybridize to one of the plurality of capture primers on the bead, and a primer extension reaction yields a capture primer extension product which is attached to the bead and includes a sequence that is complementary to the reverse primer extension product.

In embodiments that lack a fusion primer, the adapter-ligated amplified target nucleic acid hybridizes to one of the plurality of capture primers on the bead, and primer extension produces a capture primer extension product. A reverse primer hybridizes to the capture primer extension product, and a primer extension reaction produces a reverse primer extension product. The reverse primer extension product can dissociate (e.g., denature) from the capture primer extension product, and re-hybridize with a different capture primer on the same bead, for another primer extension reaction.

Repeat cycles of the RPA-bead amplification reaction yields beads that are attached with multiple copies of the adapter-ligated amplified target nucleic acids. Optionally, individual beads are attached with substantially monoclonal copies of one adapter-ligated amplified target nucleic acid. Optionally, different beads are attached with copies of a different adapter-ligated amplified target nucleic acid.

In some embodiments, the RPA-bead method includes an water-and-oil emulsion, where droplets of the aqueous reaction mixture are surrounded by an immiscible fluid (e.g., oil) so that the aqueous droplets provide compartmentalized reaction mixtures containing one or more beads that are attached with capture primers, template nucleic acids, fusion primers (or lacking fusion primers), reverse primers, polymerase, nucleotides, recombinase and accessory proteins.

In some embodiments, the capture primers are attached to a support (e.g., planar-like support) and the recombinase-polymerase reaction is conducted in a manner similar to the RPA-bead method, where the aqueous single reaction mixture contacts the surface of the support having the attached capture primers, where the aqueous single reaction mixture contains template nucleic acids, fusion primers (or lacking fusion primers), reverse primers, polymerase, nucleotides, recombinase and accessory proteins.

In some embodiments, cycles of an RPA reaction, using beads or a support, with or without an emulsion, can be conducted under isothermal amplification conditions. Examples of compositions and methods for recombinase-polymerase amplification (RPA) reactions are found in U.S. published application Nos. 2013/0225421 and 2014/0080717, and in U.S. Pat. Nos. 7,399,590, 7,666,598, 8,637,253, 8,809,021, and 9,057,097.

In some embodiments, any amplified target nucleic acids (e.g., amplicons or adapter-ligated amplified nucleic acids) that has been generated according to the present teachings, can be used as a template molecule for sequencing using any sequencing method, including sequencing-by-synthesis methods using natural nucleotides or nucleotide analogs. In some embodiments, the sequence-by-synthesis methods comprise successively incorporating nucleotides onto a terminal 3′ OH end of a primer or self-priming template, using a polymerase, detecting the incorporated nucleotides, and determining the identity of the newly incorporated nucleotide. In some embodiments, the nucleotide analogs include terminator nucleotides that are optionally reversibly blocked at the 2′ or 3′ OH sugar position on the nucleotide. For example, the reversibly blocked nucleotides include a blocking moiety attached to the 2′ or 3′ OH position via a linker that is cleavable with a chemical compound, enzyme, light or heat. The nucleotide analog can also include a detectable label (e.g., a fluorophore) attached to the 2′ or 3′ OH position, or attached to the base. Each of the four types of nucleotides, cytidine, thymidine, adenosine and guanosine, can be attached to a different label that is distinguishable from the other labels. During a sequencing-by-synthesis reaction, the polymerase incorporates the in-coming terminator nucleotide onto an existing 3′OH end, but cannot incorporate the next nucleotide until the linker is cleaved to release the blocking moiety and restore the 3′OH on the newly-incorporated nucleotide. The identity of the newly-incorporated nucleotide is determined by detecting the type of fluorophore attached to the nucleotide analog. The newly-incorporated nucleotide is treated with a cleaving agent to release the blocking moiety and restore the 3′OH. The polymerase can now incorporate another terminator nucleotide. The sequence of the template molecule is determined by performing repeated cycles of nucleotide incorporation, detection and identification of the newly incorporated nucleotide, and removal of the blocking moiety.

In some embodiments, the blocking moiety comprises an allyl, alkyl, substitute alkyl, arylalkyl, alkenyl, alkynyl, aryl, heteroaryl, acyl, cyano, alkoxy, aryloxy, or heteroaryloxy moiety. In some embodiments, the nucleotide analog includes a 3′ O allyl blocking moiety (see U.S. Pat. Nos. 8,796,432 and 7,883,869).

In some embodiments, the blocking moiety comprises —O, —S, —P, —F, —NH₂, —OCH₃, —N₃, —OPO₃, —NHCOCH₃, 2-nitrobenzene carbonate, 2,4-dinitrobenzene sulfenyl, or tetrahydrofuranyl ether (see PCT publication Nos. WO 1991/06678 Tsien, and WO 2000/053805 Stemple). In some embodiments, the nucleotide analog comprises a 3′ azido blocking moiety which is cleavable with a phosphine compound (see U.S. Pat. No. 7,635,578).

In some embodiments, the nucleotide analogs include blocking moieties attached via a disulfide linkage, acid labile linkers (e.g., dialkoxybenzyl linkers), Sieber linkers, indole linkers, or t-butyl Sieber linkers. Optionally, the linkers are cleavable linkers, and include: electrophilically-cleavable linkers, nucleophilically-cleavable linkers, photocleavable linkers, and linkers cleavable under reductive or oxidative conditions. Optionally, the linkers are cleavable via use of safety-catch linkers, and linkers cleavable by elimination mechanisms. See for example U.S. Pat. No. 7,785,796 and U.S. published application No. 2014/0106360.

In some embodiments, the nucleotide analogs include blocking moieties attached via a photocleavable linker. Optionally, the cleavable linker comprises a nitrobenzyl moiety. Optionally, the 3′ sugar position is attached to a blocking moiety, including —CH₂OCH₃ (MOM) or —CH₂CH═CH₂ (allyl). See for example, U.S. Pat. Nos. 7,713,698; 7,790,869; 8,088,575; 7,635,578; and 7,883,869.

In some embodiments, the nucleotides analogs include a detectable label attached to the base. For example, a 7-deazapurine base can be linked at the 7-position. Optionally, the linker attaching the base to the detectable label can be an acid labile linker, a photocleavable linker, disulfide linkage, dialkoxybenzyl linkers, Sieber linkers, indole linkers, or t-butyl Sieber linkers. Optionally, the linker that attaches the base to the detectable label can be cleavable under oxidation conditions, or cleavable with a palladium compound, or cleavable with thiophilic metals, including nickel, silver or mercury. In some embodiments, the terminator nucleotides also include a blocking moiety linked to the 2′ or 3′ sugar position by a linker. For example, the blocking moiety includes an azido group. In some embodiments, the linker attached to the base and the linker attached to the 2′ or 3′ sugar position are cleavable under the same conditions. See for example, U.S. Pat. Nos. 7,057,026; 7,566,537; 8,158,346; 7,541,444; 7,057,026; 7,592,435; 7,414,116; 7,427,673 and 8,399,188, and U.S. published application No. 2014/0249036.

In some embodiments, any amplified target nucleic acids (e.g., amplicons or adapter-ligated amplified nucleic acids) that has been generated according to the present teachings, can be sequenced by any sequencing method, including sequencing-by-synthesis, ion-based sequencing involving the detection of sequencing byproducts using field effect transistors (e.g., FETs and ISFETs), chemical degradation sequencing, ligation-based sequencing, hybridization sequencing, pyrophosphate detection sequencing, capillary electrophoresis, gel electrophoresis, next-generation, massively parallel sequencing platforms, sequencing platforms that detect hydrogen ions or other sequencing by-products, and single molecule sequencing platforms. In some embodiments, a sequencing reaction can be conducted using at least one sequencing primer that can hybridize to any portion of the polynucleotide constructs, including a nucleic acid adaptor or a target polynucleotide.

In some embodiments, any amplified target nucleic acids that has been generated according to the present teachings, can be sequenced using methods that detect one or more byproducts of nucleotide incorporation. The detection of polymerase extension by detecting physicochemical byproducts of the extension reaction, can include pyrophosphate, hydrogen ion, charge transfer, heat, and the like, as disclosed, for example, in U.S. Pat. No. 7,948,015 to Rothberg et al.; and Rothberg et al, U.S. Patent Publication No. 2009/0026082, hereby incorporated by reference in their entireties. Other examples of methods of detecting polymerase-based extension can be found, for example, in Pourmand et al, Proc. Natl. Acad. Sci., 103: 6466-6470 (2006); Purushothaman et al., IEEE ISCAS, IV-169-172; Anderson et al, Sensors and Actuators B Chem., 129: 79-86 (2008); Sakata et al., Angew. Chem. 118:2283-2286 (2006); Esfandyapour et al., U.S. Patent Publication No. 2008/01666727; and Sakurai et al., Anal. Chem. 64: 1996-1997 (1992).

Reactions involving the generation and detection of ions are widely performed. The use of direct ion detection methods to monitor the progress of such reactions can simplify many current biological assays. For example, template-dependent nucleic acid synthesis by a polymerase can be monitored by detecting hydrogen ions that are generated as natural byproducts of nucleotide incorporations catalyzed by the polymerase. Ion-sensitive sequencing (also referred to as “pH-based” or “ion-based” nucleic acid sequencing) exploits the direct detection of ionic byproducts, such as hydrogen ions, that are produced as a byproduct of nucleotide incorporation. In one exemplary system for ion-based sequencing, the nucleic acid to be sequenced can be captured in a microwell, and nucleotides can be flowed across the well, one at a time, under nucleotide incorporation conditions. The polymerase incorporates the appropriate nucleotide into the growing strand, and the hydrogen ion that is released can change the pH in the solution, which can be detected by an ion sensor that is coupled with the well. This technique does not require labeling of the nucleotides or expensive optical components, and allows for far more rapid completion of sequencing runs. Examples of such ion-based nucleic acid sequencing methods and platforms include the Ion Torrent PGM™ or Proton™ sequencer (Ion Torrent™ Systems, Life Technologies Corporation).

In some embodiments, amplified target nucleic acids produced using the methods, systems and kits of the present teachings can be used as a substrate for a biological or chemical reaction that is detected and/or monitored by a sensor including a field-effect transistor (FET). In various embodiments the FET is a chemFET or an ISFET. A “chemFET” or chemical field-effect transistor, is a type of field effect transistor that acts as a chemical sensor. It is the structural analog of a MOSFET transistor, where the charge on the gate electrode is applied by a chemical process. An “ISFET” or ion-sensitive field-effect transistor, is used for measuring ion concentrations in solution; when the ion concentration (such as H+) changes, the current through the transistor will change accordingly. A detailed theory of operation of an ISFET is given in “Thirty years of ISFETOLOGY: what happened in the past 30 years and what may happen in the next 30 years,” P. Bergveld, Sens. Actuators, 88 (2003), pp. 1-20.

In some embodiments, the FET may be a FET array. As used herein, an “array” is a planar arrangement of elements such as sensors or wells. The array may be one or two dimensional. A one dimensional array can be an array having one column (or row) of elements in the first dimension and a plurality of columns (or rows) in the second dimension. The number of columns (or rows) in the first and second dimensions may or may not be the same. The FET or array can comprise 102, 103, 104, 105, 106, 107 or more FETs.

In some embodiments, one or more microfluidic structures can be fabricated above the FET sensor array to provide for containment and/or confinement of a biological or chemical reaction. For example, in one implementation, the microfluidic structure(s) can be configured as one or more wells (or microwells, or reaction chambers, or reaction wells, as the terms are used interchangeably herein) disposed above one or more sensors of the array, such that the one or more sensors over which a given well is disposed detect and measure analyte presence, level, and/or concentration in the given well. In some embodiments, there can be a 1:1 correspondence of FET sensors and reaction wells.

Microwells or reaction chambers are typically hollows or wells having well-defined shapes and volumes which can be manufactured into a substrate and can be fabricated using conventional microfabrication techniques, e.g. as disclosed in the following references: Doering and Nishi, Editors, Handbook of Semiconductor Manufacturing Technology, Second Edition (CRC Press, 2007); Saliterman, Fundamentals of BioMEMS and Medical Microdevices (SPIE Publications, 2006); Elwenspoek et al, Silicon Micromachining (Cambridge University Press, 2004); and the like. Examples of configurations (e.g. spacing, shape and volumes) of microwells or reaction chambers are disclosed in Rothberg et al, U.S. patent publication 2009/0127589 and Rothberg et al, U.K. patent application GB24611127 which are hereby incorporated by reference in their entireties.

In some embodiments, the biological or chemical reaction can be performed in a solution or a reaction chamber that is in contact with, operatively coupled, or capacitively coupled to a FET such as a chemFET or an ISFET. The FET (or chemFET or ISFET) and/or reaction chamber can be an array of FETs or reaction chambers, respectively.

In some embodiments, a biological or chemical reaction can be carried out in a two-dimensional array of reaction chambers, wherein each reaction chamber can be coupled to a FET, and each reaction chamber is no greater than 10 μm³ (i.e., 1 pL) in volume. In some embodiments each reaction chamber is no greater than 0.34 pL, 0.096 pL or even 0.012 pL in volume. A reaction chamber can optionally be no greater than 2, 5, 10, 15, 22, 32, 42, 52, 62, 72, 82, 92, or 102 square microns in cross-sectional area at the top. Preferably, the array has at least 10², 10³, 10⁴, 10⁵, 10⁶, 10⁷, 10⁸, 10², or more reaction chambers. In some embodiments, at least one of the reaction chambers is operatively coupled to at least one of the FETs.

FET arrays as used in various embodiments according to the disclosure can be fabricated according to conventional CMOS fabrications techniques, as well as modified CMOS fabrication techniques and other semiconductor fabrication techniques beyond those conventionally employed in CMOS fabrication. Additionally, various lithography techniques can be employed as part of an array fabrication process.

Exemplary FET arrays suitable for use in the disclosed methods, as well as microwells and attendant fluidics, and methods for manufacturing them, are disclosed, for example, in U.S. Patent Publication No. 20100301398; U.S. Patent Publication No. 20100300895; U.S. Patent Publication No. 20100300559; U.S. Patent Publication No. 20100197507, U.S. Patent Publication No. 20100137143; U.S. Patent Publication No. 20090127589; and U.S. Patent Publication No. 20090026082, which are incorporated by reference in their entireties.

In one aspect, the disclosed methods, compositions, systems, apparatuses and kits can be used for carrying out label-free nucleic acid sequencing, and in particular, ion-based nucleic acid sequencing. The concept of label-free detection of nucleotide incorporation has been described in the literature, including the following references that are incorporated by reference: Rothberg et al, U.S. patent publication 2009/0026082; Anderson et al, Sensors and Actuators B Chem., 129: 79-86 (2008); and Pourmand et al, Proc. Natl. Acad. Sci., 103: 6466-6470 (2006). Briefly, in nucleic acid sequencing applications, nucleotide incorporations are determined by measuring natural byproducts of polymerase-catalyzed extension reactions, including hydrogen ions, polyphosphates, PPi, and Pi (e.g., in the presence of pyrophosphatase). Examples of such ion-based nucleic acid sequencing methods and platforms include the Ion Torrent PGM™ or Proton™ sequencer (Ion Torrent™ Systems, Life Technologies Corporation).

In some embodiments, the disclosure relates generally to methods for sequencing target nucleic acids that have been amplified by the teachings provided herein. In one exemplary embodiment, the disclosure relates generally to a method for obtaining sequence information from polynucleotides, comprising: (a) amplifying target polynucleotides; and (b) performing template-dependent nucleic acid synthesis using at least one of the amplified target polynucleotides produced during step (a) as a template. The amplifying can optionally be performed according to any of the multiplex amplification methods described herein.

In some embodiments, the template-dependent synthesis includes incorporating one or more nucleotides in a template-dependent fashion into a newly synthesized nucleic acid strand.

Optionally, the methods can further include producing one or more ionic byproducts of such nucleotide incorporation.

In some embodiments, the methods can further include detecting the incorporation of the one or more nucleotides into the sequencing primer. Optionally, the detecting can include detecting the release of hydrogen ions.

In another embodiment, the disclosure relates generally to a method for sequencing a nucleic acid, comprising: (a) amplifying target polynucleotides according to the methods disclosed herein; (b) disposing the amplified polynucleotides (e.g., amplicons or adapter-ligated amplified nucleic acids) into a plurality of reaction chambers, wherein one or more of the reaction chambers are in contact with a field effect transistor (FET). Optionally, the method further includes contacting amplified nucleic acids which are disposed into one of the reaction chambers, with a polymerase thereby synthesizing a new nucleic acid strand by sequentially incorporating one or more nucleotides into a nucleic acid molecule. Optionally, the method further includes generating one or more hydrogen ions as a byproduct of such nucleotide incorporation. Optionally, the method further includes detecting the incorporation of the one or more nucleotides by detecting the generation of the one or more hydrogen ions using the FET.

In some embodiments, the detecting includes detecting a change in voltage and/or current at the at least one FET within the array in response to the generation of the one or more hydrogen ions.

In some embodiments, the FET can be selected from the group consisting of: ion-sensitive FET (isFET) and chemically-sensitive FET (chemFET).

One exemplary system involving sequencing via detection of ionic byproducts of nucleotide incorporation is the Ion Torrent PGM™ or Proton™ sequencer (Life Technologies), which is an ion-based sequencing system that sequences nucleic acid templates by detecting hydrogen ions produced as a byproduct of nucleotide incorporation. Typically, hydrogen ions are released as byproducts of nucleotide incorporations occurring during template-dependent nucleic acid synthesis by a polymerase. The Ion Torrent PGM™ or Proton™ sequencer detects the nucleotide incorporations by detecting the hydrogen ion byproducts of the nucleotide incorporations. The Ion Torrent PGM™ or Proton™ sequencer can include a plurality of nucleic acid templates to be sequenced, each template disposed within a respective sequencing reaction well in an array. The wells of the array can each be coupled to at least one ion sensor that can detect the release of H⁺ ions or changes in solution pH produced as a byproduct of nucleotide incorporation. The ion sensor comprises a field effect transistor (FET) coupled to an ion-sensitive detection layer that can sense the presence of H⁺ ions or changes in solution pH. The ion sensor can provide output signals indicative of nucleotide incorporation which can be represented as voltage changes whose magnitude correlates with the H⁺ ion concentration in a respective well or reaction chamber. Different nucleotide types can be flowed serially into the reaction chamber, and can be incorporated by the polymerase into an extending primer (or polymerization site) in an order determined by the sequence of the template. Each nucleotide incorporation can be accompanied by the release of H⁺ ions in the reaction well, along with a concomitant change in the localized pH. The release of H⁺ ions can be registered by the FET of the sensor, which produces signals indicating the occurrence of the nucleotide incorporation. Nucleotides that are not incorporated during a particular nucleotide flow may not produce signals. The amplitude of the signals from the FET can also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers can permit the instrument to resolve the sequence of many nucleic acid templates simultaneously. Further details regarding the compositions, design and operation of the Ion Torrent PGM™ or Proton™ sequencer can be found, for example, in U.S. patent application Ser. No. 12/002,781, now published as U.S. Patent Publication No. 2009/0026082; U.S. patent application Ser. No. 12/474,897, now published as U.S. Patent Publication No. 2010/0137143; and U.S. patent application Ser. No. 12/492,844, now published as U.S. Patent Publication No. 2010/0282617, all of which applications are incorporated by reference herein in their entireties.

It is well known in the art that erroneous conclusions can arise from data obtained from a molecular biology workflow that is used to detect the number or amount of amplicons containing a sequence derived from a target polynucleotide. The errors are known to arise from any step of the workflow, including extraction of a heterogeneous cell source, contaminant sequences in an enriched nucleic acid sample, mis-priming events during the reverse transcription and/or amplification steps, spurious primer extension products, and sequencing errors. In some embodiments, the plurality of amplicons that are produced by any of the present teachings can yield data (e.g., quantifying or counting data) which represents an approximation of the complexity and abundance of different transcripts that are present in a starting RNA or DNA sample. In some embodiments, the plurality of amplicons that are produced by any of the present teachings may not represent an absolutely accurate count of RNA or DNA sequences of interest in a sample. In some embodiments, the quantifying step can yield data that differs from the actual quantity of RNA or DNA sequences of interest present in a sample by about 0.01-0.1%, or about 0.1-0.5%, or about 0.5-1%, or about 1-2.5%, or about 2.5-5%, or about 5-7.5%, or about 7.5-10%, or about 10-25%, or more.

In some embodiments, the plurality of target polynucleotides in the reaction mixture comprises sequences derived from RNA in the sample. For example, the plurality of target polynucleotides in the reaction mixture can include a plurality of cDNAs that individually correspond to one or more RNA sequences.

In some embodiments, generating the plurality of polynucleotides comprises converting RNA to cDNA using any suitable means. In some embodiments, generating the plurality of polynucleotides comprises: conducting a reverse transcription reaction with RNA and plurality of primers to generate plurality of cDNA. In some embodiments, the plurality of primers used in the reverse transcription reaction comprises random sequence primers. In some embodiments, the reverse transcription reaction includes at least one enzyme having RNA-dependent DNA polymerase activity. In some embodiments, the enzyme having RNA-dependent DNA polymerase activity also has DNA-dependent DNA polymerase activity. In some embodiments, the plurality of cDNA includes polyA cDNA and non-polyA cDNA. In some embodiments, the plurality of cDNA includes a plurality of first strand cDNA, a plurality of second strand cDNA, or a plurality of first and second strand cDNA. In some embodiments, the plurality of target polynucleotides (e.g., cDNA) can be generated by reverse transcribing a plurality of different RNA sequences in a sample, using a plurality of random sequence primers, at least one enzyme having RNA-dependent DNA polymerase activity, and a plurality of nucleotides, under conditions suitable for generating at least a first strand cDNA.

In some embodiments, the reverse transcribing can be conducted by directly ligating the RNA to a plurality of double-stranded RNA/DNA or DNA/DNA adaptors, heating to remove one strand of the double-stranded adaptors, and conducting a reverse transcription reaction with primers that hybridize at least one adaptor sequence. In some embodiments, the reverse transcribing can be conducted according to an RNA-Seq procedure described in U.S. Pat. No. 8,192,941, which is incorporated by reference in its entirety. In some embodiments, the plurality of target polynucleotides (e.g., cDNA) can be generated by and RNA-Seq method (see for example U.S. Pat. No. 8,192,941, incorporated by reference in its entirety), which can include: ligating double-stranded adaptors to both ends of single-stranded RNA, removing one of the strands of both double-stranded adaptors by denaturation to form an RNA molecule having single-stranded adaptors appended to both ends, hybridizing the RNA with an extendible primer that hybridizes to at least one of the single-stranded adaptors that is appended to the RNA, and conducting a reverse transcription reaction with an RNA-dependent DNA polymerase, and a plurality of nucleotides. In some embodiments, the double-stranded adaptors can include DNA/RNA or DNA/DNA. In some embodiments, the double-stranded adaptors are ligated to the RNA ends with an RNA ligase.

In some embodiments, the disclosure relates generally to methods, compositions, systems, apparatuses and kits for providing a reverse transcription reaction mixture containing one or more RNA sequence. In some embodiments, the reverse transcription reaction mixture further includes any one or any combination of a plurality of primers, at least one enzyme having RNA-dependent DNA polymerase activity and/or a plurality of nucleotides.

In some embodiments, the reverse transcription reaction mixture further includes RNase H to degrade the RNA during or after the reverse transcription step.

In some embodiments, the reverse transcription reaction mixture further includes any one or any combination of compounds: magnesium, manganese, formamide, DMSO, betaine, trehalose, spermidine, sulfones, sodium pyrophosphate, low molecular amides, single-stranded binding proteins and/or an archaeal accessory factor that enhances the activity of an RNA-dependent DNA polymerase or a DNA-dependent DNA polymerase. In some embodiments, the reverse transcription reaction mixture can be incubated under isothermal, thermal-cycling, or a combination of both temperature conditions.

In some embodiments, a reverse transcription reaction further includes an external RNA control to permit characterization of the starting RNA sample against a defined performance criteria. In some embodiments, the external control RNA comprises a known RNA sequence (e.g., beta-actin, glyceraldehydes-3-phosphate dehydrogenase, or rRNA) or a commercially-available ERCC Spike-In Control mix (Ambion™).

In some embodiments, the disclosure relates generally to methods, compositions, systems, apparatuses and kits for reverse transcribing RNA by contacting at least one RNA molecule with any one or any combination of a plurality of primers, an enzyme having RNA-dependent DNA polymerase activity and/or a plurality of nucleotides. In some embodiments, the at least one RNA molecule, the plurality of primers, the enzyme having RNA-dependent DNA polymerase activity and the plurality of nucleotides can be contacted together substantially simultaneously, or sequentially, in any combination and in any order. In some embodiments, the plurality of primers comprises a plurality of random sequence primers, target-specific primers, or polyT primers. In some embodiments, the contacting can be conducted in a single reaction mixture or in separate reaction mixtures.

In some embodiments, the disclosure relates generally to methods, compositions, systems, apparatuses and kits for reverse transcribing RNA by hybridizing at least one RNA molecule with a plurality of primers to form at least one RNA/primer complex. In some embodiments, at least one of the plurality of primers can hybridize to at least a portion of one or more RNA molecules. In some embodiments, the hybridizing is conducted in a single reaction mixture (e.g., a reverse transcription reaction mixture).

In some embodiments, the disclosure relates generally to methods, compositions, systems, apparatuses and kits for converting RNA to cDNA by conducting a reverse transcription reaction. In some embodiments, the reverse transcription reaction generates a plurality of cDNA that represents a whole transcriptome, or represents a portion of RNA sequences in a whole transcriptome. In some embodiments, the reverse transcription reaction comprises at least one RNA molecule, a plurality of primers, an enzyme having RNA-dependent DNA polymerase activity, and a plurality of nucleotides. In some embodiments, the reverse transcription reaction includes hybridizing the RNA with the plurality of primers to form a plurality of RNA/primer complexes. In some embodiments, the reverse transcription reaction includes incorporating one nucleotide onto a primer that is part of the RNA/primer complex. In some embodiments, the nucleotide is incorporated onto the primer in a template-based manner, which can include complementary base pairing, including standard A-T or C-G base pairing, or optionally other forms of base-pairing interactions. In some embodiments, the primer extension reaction includes successively incorporating nucleotides onto a primer that is part of an RNA/primer complex. In some embodiments, the primer extension reaction can be conducted in a single reaction mixture.

In some embodiments, the RNA can be naturally-occurring, recombinant or synthetically-prepared. In some embodiments, the RNA includes any one or any combination of total RNA, or RNA enriched for one or more RNA species, non-enriched RNA, coding RNA, non-coding RNA, polyA RNA or non-polyA RNA. In some embodiments, the RNA can be isolated from a single fresh or archived cell, fresh cells, fresh tissues, or archived cells or tissues that are formalin-treated and/or embedded in paraffin or plastic, or cells or tissues that are formalin fixed paraffin-embedded (FFPE). In some embodiments, the RNA can be isolated from any source including from organisms such as prokaryotes, eukaryotes (e.g., humans, plants and animals), fungus, and viruses; cells; tissues; normal or diseased cells or tissues or organs, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods. In some embodiments, the RNA can be unfragmented, or fragmented by mechanical force, chemical, enzyme or heat. In some embodiments, the RNA can be depleted of one or more species such as rRNA. In some embodiments, the RNA comprises any one or any combination of any type of RNA, including: total RNA, mRNA, polyA RNA, polysomal RNA, tRNA, ribosomal RNA, lincRNA, miRNA, piRNA, siRNA, SRP RNA, tmRNA, snRNA, snoRNA, SmY RNA, scaRNA, gRNA, aRNA, crRNA, tasiRNA, rasiRNA and 7SKRNA.

In some embodiments, the disclosure relates generally to methods, compositions, systems, apparatuses and kits, comprising conducting a reverse transcription reaction with about 200 pg-10 ng of RNA, or about 10 ng-100 ng of RNA, or about 100 ng-500 ng of RNA, or about 500 ng-1 μg, or more, of RNA. Optionally, the RNA can be isolated from an unfixed cells or tissues, or from an FFPE sample.

In some embodiments, an external RNA control can be added to the RNA sample permit characterization of the starting RNA against defined performance criteria. For example, addition of an external RNA control can enable measurement of absolute abundance of an RNA sequence of interest. In some embodiments, the external control RNA comprises a known RNA sequence (e.g., beta-actin, glyceraldehydes-3-phosphate dehydrogenase, or rRNA) or a commercially-available ERCC Spike-In Control mix (Ambion™).

In some embodiments, the disclosure relates generally to methods, compositions, systems, apparatuses and kits, where the enzyme employed in the reverse transcription reaction comprises a polymerase. In some embodiments, the enzyme employed in the reverse transcription reaction comprises RNA-dependent DNA polymerase activity. In some embodiments, the enzyme employed in the reverse transcription reaction also has DNA-dependent DNA polymerase activity. In some embodiments, the enzyme having RNA-dependent DNA polymerase activity also has strand-displacement activity. In some embodiments, the enzyme employed in the reverse transcription reaction comprises a wild-type, mutant, or chimeric enzyme. In some embodiments, the enzyme employed in the reverse transcription reaction has RNase H activity, or lacks or exhibits reduced RNase H activity. In some embodiments, the enzyme employed in the reverse transcription reaction exhibits increased thermostability. In some embodiments, the enzyme employed in the reverse transcription reaction exhibits high fidelity. In some embodiments, the enzyme employed in the reverse transcription reaction comprises a reverse transcriptase enzyme.

In some embodiments, the reverse transcription reaction can be conducted with any one or any combination of reverse transcriptases, including: Moloney murine leukemia virus (M-MLV) reverse transcriptase; human immunodeficiency virus (HIV) reverse transcriptase; rous sarcoma virus (RSV) reverse transcriptase; avian myeloblastosis virus (AMV) reverse transcriptase; rous associated virus (RAV) reverse transcriptase; myeloblastosis associated virus (MAV) reverse transcriptase or other avian sarcoma-leukosis virus (ASLV) reverse transcriptases.

In some embodiments, the enzyme employed in the reverse transcription reaction comprises a mutant M-MLV reverse transcriptase that exhibits reduced Rnase H activity and is high fidelity (U.S. Pat. No. 7,056,716 which is hereby incorporated by reference in its entirety).

In some embodiments, the enzyme employed in the reverse transcription reaction comprises a mutant M-MLV reverse transcriptase that exhibits reduced terminal deoxynucleotidyl transferase activity (U.S. Pat. No. 8,541,219 which is hereby incorporated by reference in its entirety).

In some embodiments, the enzyme employed in the reverse transcription reaction comprises a mutant M-MLV reverse transcriptase that exhibits reduced terminal deoxynucleotidyl transferase activity, or increased thermostability, or increased fidelity (U.S. Pat. No. 7,078,208 which is hereby incorporated by reference in its entirety).

In some embodiments, the enzyme employed in the reverse transcription reaction comprises a mutant M-MLV reverse transcriptase that exhibits reduced terminal deoxynucleotidyl transferase activity, or increased thermostability, or increased fidelity (U.S. Pat. No. 8,753,845 which is hereby incorporated by reference in its entirety).

In some embodiments, the enzyme employed in the reverse transcription reaction comprises a hyperactive reverse transcriptase having reduced RNase H activity (U.S. Pat. No. 8,361,754 which is hereby incorporated by reference in its entirety).

In some embodiments, the disclosure relates generally to methods, compositions, systems, apparatuses and kits, include a plurality of primers for conducting a reverse transcription reaction, where the primers comprise random-sequence primers. In some embodiments, a plurality of random-sequence primers can be used to generate cDNA in a reverse transcription reaction. In some embodiments, a random-sequence primer comprises DNA, RNA or DNA/RNA. In some embodiments, at least one of the random-sequence primers, in the plurality, can hybridize to any portion of any type of RNA. In some embodiments, the random-sequence primers have extendible 3′ ends.

In some embodiments, a reverse transcription reaction can be conducted by contacting RNA with a plurality of random-sequence primers, an enzyme having RNA-dependent DNA polymerase activity, and a plurality of nucleotide (or analogs thereof). Optionally, the reverse transcription reaction can be conducted with a mixture of random-sequence primers and target-specific primers.

In some embodiments, a random-sequence primer comprises an oligonucleotide that generally includes a sequence that is based on a statistical expectation, or an empirical observation, that the sequence of the random primer is hybridizable to one or more target sequences in a plurality of nucleic acids. In some embodiments, the random sequence primer is not necessarily based on a particular or specific sequence of a nucleic acid.

In some embodiments, a random-sequence primer comprises an oligonucleotide having a random sequence in which the nucleotides at any given position along the oligonucleotide can be any of the five deoxyribonucleotides (A, T, C, G or U) or analogs thereof. In some embodiments, the sequence of a random-sequence primer (or its complementary sequence) can be naturally-occurring, recombinant or synthesized by chemical synthesis methods. In some embodiments, the sequence of a random-sequence primer (or its complementary sequence) or may or may not be present in a plurality of nucleic acids. In some embodiments, a random-sequence primer comprises a randomly generated sequence. In some embodiments, the order of nucleotides in a random-sequence primer can be selected at random from two or more different nucleotides. In some embodiments, all possible sequence combinations of the nucleotides selected at random may be represented in a collection of random-sequence primers. In some embodiments, generation of one or more random primers does not include a step of excluding or selecting certain sequences or nucleotide combinations from the possible sequence combinations in the random portion of the one or more random primers.

In some embodiments, a random-sequence primer can include a random sequence that is located in the 3′ or 5′ portion, or an internal portion of the random-sequence primer. A random-sequence primer can include any homo-polymer sequence (e.g., polyA, polyG, polyC, polyT or polyU).

In some embodiments, a plurality of random-sequence primers comprises two or more different sequences.

In some embodiments, at least one random-sequence primer can hybridize to a region of an RNA molecule in a sample of a plurality of RNA molecules. In some embodiments, the 3′ end of the random-sequence primers can hybridize to a portion of an RNA molecule. In some embodiments, the entire length of the random-sequence primers can hybridize to a portion of an RNA molecule.

In some embodiments, a plurality of random-sequence primers contains a collection of random-sequence primers having the same or different sequences. In some embodiments, at least one of the random-sequence primers in the plurality can hybridize to at least one target sequence. In some embodiments, different random-sequence primers in the plurality can hybridize to different target sequences. A random-sequence primer can hybridize to a plurality of different sites on a target nucleic acid. In some embodiments one portion of a random-sequence primer includes a random sequence, and another portion of includes a defined sequence.

In some embodiments, a random-sequence primer comprises a tailed primer. In some embodiments, a tailed primer includes a 3′-region having a random sequence that hybridizes to a target nucleic acid molecule, and a 5′-region that is a non-hybridizing sequence. In some embodiments, the non-hybridizing portion of a tailed primer includes non-random sequence. In some embodiments, the 3′-region of a random-sequence primer includes a random sequence in combination with a region that comprises poly-T sequences. In some embodiments, the 5′ non-hybridizing portion of a tailed primer can be about 2-10, or about 10-20, or about 20-30, or about 30-50 nucleotides in length.

In some embodiments, the random-sequence primers are about 4 or 5 bases, or 6-10 bases, or about 10-15 bases, or about 15-20 bases, or about 20-25 bases, or about 25-30 bases in length, or longer. In some embodiments, the random-sequence primers can be up to about 100 bases in length.

In some embodiments, the random-sequence primers comprise pentameric, hexameric, heptameric, octomeric, nonameric, decameric, or higher order lengths of oligonucleotide primers.

In some embodiments, the reverse transcribing reactions employ single-stranded or double-stranded nucleic acid primers. In some embodiments, the reverse transcribing reactions employ DNA, RNA or DNA/RNA hybrid primers.

In some embodiments, the reverse transcribing reactions produce a plurality of first strand cDNA products, a plurality of second strand cDNA products, and/or a plurality of first and second strand cDNA products.

In some embodiments, the reverse transcribing reactions include RNA that is naturally-occurring, recombinant, synthetically-prepared, or any combination of these types of RNA.

In some embodiments, the reverse transcribing reactions include RNA that comprises total RNA, RNA enriched for one or more RNA species, or non-enriched RNA.

In some embodiments, the reverse transcribing reactions include a plurality of primers that comprise DNA, RNA or DNA/RNA hybrid oligonucleotides. In some embodiments, the plurality of primers comprises single-stranded or double-stranded primers. In some embodiments, at least one of the primers in the plurality of primers comprises a sequence that can hybridize to at least a portion of the one or more RNA. In some embodiments, plurality of primers comprises any one or any combination of: random-sequence primers, target-specific primers, homo-polymer primers (e.g., polyA, polyT, polyG, polyC or polyU primers), labeled primers, non-labeled primers, and/or non-extendible primers. Optionally, the non-extendible primers includes a 3′ end linked to at least one blocking group that inhibits or blocks primer extension by a polymerase.

In some embodiments, the reverse transcribing reactions comprise a plurality of random sequence primers and can generate a plurality of different cDNAs that correspond to polyA RNA and non-polyA RNA sequences.

In some embodiments, the reverse transcribing reactions include an enzyme having RNA-dependent DNA polymerase activity and RNase H activity, or the enzyme has reduced or lacks RNase H activity. In some embodiments, the enzyme having RNA-dependent DNA polymerase activity can also include DNA-dependent DNA polymerase activity, or reduced or lack DNA-dependent DNA polymerase activity. In some embodiment, the enzyme having RNA-dependent DNA polymerase activity can be derived from a viral, retroviral, prokaryote or eukaryote source. In some embodiment, the enzyme having RNA-dependent DNA polymerase activity can be a heat-labile enzyme or can exhibit improved thermal-stability.

In some embodiments, the reverse transcribing reactions include a plurality of nucleotides includes deoxyribonucleotides, ribonucleotides, modified deoxyribonucleotides or modified ribonucleotides. In some embodiments, the plurality of nucleotides comprises a purine and/or pyrimidine base, including adenine, guanine, cytosine, thymine or uracil.

In some embodiments, the reverse transcribing reactions can be conducted at a temperature range of about 20-60° C. For example, temperature ranges above approximately 42° C. are useful for reducing secondary structures that can form in RNA. Temperature ranges of lower than about 42° C. are useful when employing random sequence primers.

In some embodiments, a transcription step (e.g., in vitro transcription) can precede the reverse transcription step.

In some embodiments, the reverse transcribing reaction and the multiplex nucleic acid amplification reaction can be conducted in a single reaction vessel. Optionally, the reverse-transcribing reaction can be conducted in a first reaction vessel, and the multiplex nucleic acid amplification reaction can be conducted in a second reaction vessel. Optionally, the reverse-transcribing reaction can be conducted in a first single reaction mixture. Optionally, the multiplex nucleic acid amplification reaction can be conducted in a second single reaction mixture. Optionally, the reverse transcribing reaction can be conducted in a first reaction mixture, and additional reagents can be added to the first reaction mixture to conduct the multiplex nucleic acid amplification reaction.

In some embodiments, the disclosure relates generally to compositions, methods, systems, apparatuses and kits, comprising a kit which contains a plurality of target-specific primers.

In some embodiments, the target-specific primers in a kit are complementary or identical to at least a portion of one or more target polynucleotides containing sequences derived from one or more expressed genes a single cell or from a plurality of cells.

In some embodiments, the target-specific primers in a kit are complementary or identical to at least a portion of one or more target polynucleotides that contain sequences derived from one or more expressed genes in a non-diseased cell, a cancer cell, an ooctye, an embryo, a stem cell, or a cell exposed to a companion diagnostic compound.

In some embodiments, the kit contains at least 1000, 2500, 5000, 7500, 10,000, 12,000, 15,000, 17,500, 20,000, 25,000, 50,000, 100,000, 200,000 or 500,000 different target-specific primers.

In some embodiments, the kit contains at least 1000, 2500, 5000, 7500, 10,000, 12,000, 15,000, 17,500, 20,000, 25,000, 50,000, 100,000, 200,000 or 500,000 different target-specific primer pairs.

Optionally, at least one primer in the plurality of target-specific primers in the kit contains at least one cleavable group.

Optionally, each of the plurality of target-specific primers in the kit contains at least one cleavable group.

Optionally, the cleavable group can be 8-oxo-deoxyguanosine, deoxyuridine or bromodeoxyuridine.

In some embodiments, the kits further comprise a cleaving agent capable of cleaving the at least one cleavable group of the plurality of target specific primers.

Optionally, the cleaving agent includes RNaseH, uracil DNA glycosylase, Fpg or alkali.

Optionally, the cleaving agent includes uracil DNA glycosylase.

In some embodiments, the kits further comprise at least one polymerase.

Optionally, the at least one DNA polymerase includes a thermostable or thermal labile DNA polymerase.

In some embodiments, the kits further comprise a plurality of nucleotides.

In some embodiments, the kits further comprise a ligase. Optionally, the ligase includes RNA or DNA ligase.

In some embodiments, the kits further comprise one or more adaptors.

Optionally, the one or more adaptors in the kit are not complementary or identical to the 5′ end of the plurality of target-specific primers.

Optionally, the one or more adapters in the kit do not include a nucleic acid sequence that is complementary or identical to the terminal 10 nucleotides at the 5′ end of the plurality of target-specific primers.

Optionally, the one or more adapters in the kit comprise a universal priming sequence, a tag, or a unique identifier sequence (e.g., barcode sequence).

Optionally, the universal priming sequence comprises an amplification priming sequence or a sequencing priming sequence.

Optionally, at least one of the one or more adaptors in the kit is phosphorylated at the 5′ end.

Optionally, a plurality of the one or more adaptors in the kit is single-stranded or double-stranded.

Optionally, the kit further comprises reagents, which can include any one or any combination of: magnesium, manganese, calcium, potassium, dithiothreitol (DTT), glycerol, spermidine, and/or BSA (bovine serum albumin), formamide, DMSO, betaine, trehalose, sulfones, sodium pyrophosphate, low molecular amides, and/or single-stranded binding proteins.

Optionally, each of the components of the kit can be provided in a separate container or vessel, or any combination of a mixture of different components can be provided in one or several containers.

Optionally, any one or more than one component of the kit can be provided in dry form, including in crystallized, freeze-dried, lyophilized form.

Optionally, any one or more than one component in the kit can be provided in solution, including in an aqueous solution.

In some embodiments, the components of the kit including any one or any combination of: primers (e.g., a plurality of target-specific primers and/or random sequence primers), polymerase, plurality of nucleotides, cleaving agent, reverse transcriptase, adaptors (e.g., DNA/DNA or RNA/DNA adaptors), RNA and/or RNA ligase, magnesium, manganese, calcium, potassium, dithiothreitol (DTT), glycerol, spermidine, and/or BSA (bovine serum albumin), formamide, DMSO, betaine, trehalose, sulfones, sodium pyrophosphate, low molecular amides, and/or single-stranded binding proteins. In some embodiments, the kit is provided to perform multiplex PCR in a single reaction chamber or vessel.

In some embodiments, the plurality of target polynucleotides within the reaction mixture includes, or is otherwise derived from, DNA or RNA isolated from a sample (e.g., a biological sample). The biological sample optionally includes a single cell, a plurality of cells, a cell culture, cell lysate, a tissue, an organ, bodily fluid (including but not limited to urine, stool, saliva, blood, plasma, serum, lymph, cerebrospinal fluid, and cell or tissue exudate). In some embodiments, the plurality of polynucleotides can be extracted from DNA or RNA, cells or tumors circulating in any bodily fluid. Optionally, the bodily fluid includes blood, urine, serum, lymph, tumor, saliva, anal and vaginal secretions, amniotic samples, perspiration, and semen. In some embodiments, the plurality of target polynucleotides in the reaction mixture includes at least some polynucleotides synthesized in vitro (e.g., in vitro transcription). In some embodiments, the biological sample includes a single cell. In some embodiments, the biological sample includes fetal cells or fetal DNA extracted from maternal tissue or blood taken from a pregnant woman. In some embodiments, at least some of the plurality of target polynucleotides are extracted or otherwise derived from a biological sample containing at least one cell or bodily fluid.

In some embodiments, the disclosure relates generally to methods, compositions, systems, apparatuses and kits for amplifying one or more target polynucleotides derived from a single source, such as cDNA or RNA. In some embodiments, the one or more target polynucleotides are derived from RNA obtained from a single cell. In another embodiment, the one or more target polynucleotides derived from RNA are obtained from a population of cells. In one embodiment, the RNA derived from a population of cells is obtained from a cancer cell, oocyte, embryo, stem cell, or a cell exposed to a companion diagnostic compound. In some embodiments, the one or more target polynucleotides include cDNA that is reverse transcribed from a RNA transcriptome. In one embodiment, the one or more target polynucleotides include cDNA that is reverse transcribed from a RNA transcriptome and the plurality of target polynucleotides are representative or indicative of the level of mRNA expression of one or more active genes in the RNA transcriptome. In yet another embodiment, the one or more target polynucleotides include a cDNA population that represents mRNA expression in the RNA transcriptome.

In some embodiments, the sample contains a single type of nucleic acid or a mixture of different types of nucleic acids. In some embodiments, the sample contains a plurality of nucleic acids having the same sequence or different sequences. In some embodiments, the sample contains single-stranded or double-stranded nucleic acids. In some embodiments, the sample contains RNA, cDNA or DNA. In some embodiments, the sample contains a plurality of nucleic acids that are naturally-occurring, recombinant or synthetically-prepared. In some embodiments, the sample contains nucleic acids that are isolated from a single fresh or archived cell, fresh cells, fresh tissues, or archived cells or tissues that are formalin-treated and/or embedded in paraffin or plastic, or cells or tissues that are formalin fixed paraffin-embedded (FFPE). In some embodiments, the sample contains nucleic acids that are isolated from any source including from organisms such as prokaryotes, eukaryotes (e.g., humans, plants and animals), fungus, and viruses; cells; tissues; normal or diseased cells or tissues or organs, body fluids including blood, urine, serum, lymph, tumor, saliva, anal and vaginal secretions, amniotic samples, perspiration, and semen; environmental samples; culture samples; or synthesized nucleic acid molecules prepared using recombinant molecular biology or chemical synthesis methods.

In some embodiments, the sample contains nucleic acids that are unfragmented, or fragmented by mechanical force, chemical, enzyme or heat. In some embodiments, the sample contains nucleic acids that are depleted of one or more nucleic acid species.

In some embodiments, the sample includes polynucleotides derived from whole-genome amplification (WGA) of genomic DNA extracted from a single cell, multiple cells, whole tissue, blood or other bodily fluid. Optionally, the single cell is taken from a fertilized zygote, blastocyst or embryo, or is a fetal cell extracted from maternal tissue or blood, or is a tumor cell (e.g., a circulating tumor cell).

In some embodiments, the plurality of target polynucleotides (e.g., in the single reaction mixture) comprises a plurality of single-stranded or double-stranded nucleic acids derived from one or more cells. In some embodiments, the plurality of target polynucleotides comprises RNA, DNA, or cDNA derived from one or more cells. In some embodiments, the DNA can be isolated from a naturally-occurring source, recombinant, or synthesize by a chemical synthesis procedure. In some embodiments, the cDNA can be derived from RNA. In some embodiments, the plurality of target polynucleotides comprises first strand cDNA, second strand cDNA, or both first and second strand cDNA. In some embodiments, the plurality of target polynucleotides comprises single-stranded or double-stranded cDNA. Optionally, the single-stranded or double-stranded cDNA can be generated from RNA. Optionally, the RNA can be isolated from one or more cells or the plurality of RNA can be generated by an in vitro transcription procedure. In some embodiments, any reverse transcription reaction can be used to generate the plurality of cDNA.

In some embodiments, the plurality of target polynucleotides includes any one or any combination of wild-type sequences, mutant sequences, fusion sequences, splice isoforms, allelic variants, and/or single nucleotide variants. In some embodiments, the relative abundance of the different target polynucleotide sequences, in the plurality of target polynucleotides, reflects the abundances of different polynucleotide sequences present in a whole transcriptome, or in a portion of a whole transcriptome.

In another embodiment, the composition (as well as related methods, systems, apparatuses and kits) includes a plurality target polynucleotides where at least one of the target polynucleotides includes at least one mutational hotspot, single nucleotide polymorphism (SNP), short tandem repeat (STR), genetic variant, genetic rearrangement (such as a translocation, deletion, insertion, duplication, truncation, copy number variation), coding region, splice variant, RNA transcript, RNA transcript fusion, exon or gene.

In some embodiments, the composition (as well as related methods, systems, apparatuses and kits) includes a plurality of target polynucleotides where at least 5%, 10%, 20%, 30%, 40%, 50%, 60%, 70%, 80%, 90%, 95%, 98%, 99% or more of the target polynucleotides include at least one mutational hotspot, single nucleotide polymorphism (SNP), short tandem repeat (STR), genetic variant, genetic rearrangement (such as a translocation, deletion, insertion, duplication, truncation, copy number variation), coding region, splice variant, RNA transcript, RNA transcript fusion, exon or gene. In some embodiments, one or more of the target polynucleotides include a copy number variation. In another embodiment, one or more of the target polynucleotides include an RNA transcript or splice variant. In yet another embodiment, the plurality of target polynucleotides includes RNA transcripts. In yet another embodiment, the plurality of target polynucleotides can be derived from a cell population. In one embodiment, the plurality of target polynucleotides can be derived from RNA of a single cell. In another embodiment, the plurality of target polynucleotides can be formed by reverse transcription of RNA extracted from a cell population. In yet another embodiment, the plurality of target polynucleotides can include a plurality of cDNA formed via reverse transcription of total mRNA extracted from a cell population. In another embodiment, the total mRNA extracted from a cell population can include an aberrant transcript within the RNA transcriptome and the plurality of target polynucleotides includes a cDNA derived from the aberrant transcript. In yet another embodiment, the aberrant transcript can be associated with cancer. In yet another embodiment, the aberrant transcript can include a splice transcript within the RNA transcriptome and the plurality of target polynucleotides includes a cDNA derived from the aberrant splice transcript. In some embodiments, the aberrant splice transcript is associated with cancer.

In some embodiments, the composition includes a plurality of target polynucleotides derived from RNA that are associated with at least one mutation associated with cancer, and where the mutation is located in at least one of the genes selected from: ABI1; ABL1; ABL2; ACSL3; ACSL6; AFF1; AFF3; AFF4; AKAP9; AKT1; AKT2; ALK; APC; ARHGAP26; ARHGEF12; ARID1A; ARNT; ASPSCR1; ASXL1; ATF1; ATIC; ATM; AXIN2; BAP1; BARD1; BCAR3; BCL10; BCL11A; BCL11B; BCL2; BCL3; BCL6; BCL7A; BCL9; BCR; BIRC3; BLM; BMPR1A; BRAF; BRCA1; BRCA2; BRD3; BRD4; BRIP1; BUB1B; CARD11; CARS; CASC5; CBFA2T3; CBFB; CBL; CBLB; CBLC; CCDC6; CCNB1iP1; CCND1; CCND2; CD74; CD79A; CDC73; CDH1; CDH11; CDK4; CDK6; CDKN2A; CDKN2B; CDKN2C; CDX2; CEBPA; CEP110; CHEK1; CHEK2; CHIC2; CHN1; CIC; CIITA; CLP1; CLTC; CLTCL1; COL1A1; CREB1; CREB3L2; CREBBP; CRTC1; CRTC3; CSF1R; CTNNB1; CXCR7; CYLD; CYTSB; DCLK3; DDB2; DDIT3; DDR2; DDX10; DDX5; DDX6; DEK; DGKG; DICER1; DNMT3A; EGFR; EIF4A2; ELF4; ELL; ELN; EML4; EP300; EPS15; ERBB2; ERBB4; ERC1; ERCC2; ERCC3; ERCC4; ERCC5; ERG; ETV1; ETV4; ETV5; ETV6; EWSR1; EXT1; EXT2; EZH2; FAM123B; FANCA; FANCC; FANCD2; FANCE; FANCF; FANCG; FAS; FBXW7; FCRL4; FGFR1; FGFR1OP; FGFR2; FGFR3; FH; FIP1L1; FLCN; FLI1; FLT1; FLT3; FNBP1; FOXL2; FOXO1; FOXO3; FOXO4; FOXP1; FUS; GAS7; GATA1; GATA2; GATA3; GMPS; GNAQ; GNAS; GOLGA5; GOPC; GPC3; GPHNGPR124; HIP1; HIST1H4I; HLF; HNF1A; HNRNPA2B1; HOOK3; HOXA11; HOXA13; HOXA9; HOXC11; HOXC13; HOXD13; HRAS; HSP90AA1; HSP90AB1; IDH1; IDH2; IKZF1; IL2; IL21R; IL6ST; IRF4; ITGA10; ITGA9; ITK; JAK1; JAK2; JAK3; KDM5A; KDM5C; KDM6A; KDR; KDSR; KIAA1549; KIT; KLF6; KLK2; KRAS; KTN1; LASP1; LCK; LCP1; LHFP; LIFR; LMO2; LPP; MAF; MALT1; MAML2; MAP2K1; MAP2K4; MDM2; MDM4; MECOM; MEN1; MET; MITF; MKL1; MLH1; MLL; MLLT1; MLLT10; MLLT3; MLLT4; MLLT6; MN1; MPL; MRE11A; MSH2; MSH6; MSI2; MSN; MTCP1; MTOR; MUC1; MYB; MYC; MYCL1; MYCN; MYH11; MYH9; MYST3; MYST4; NACA; NBN; NCOA1; NCOA2; NCOA4; NEK9; NF1; NF2; NFE2L2; NFKB2; NIN; NKX2-1; NLRP1; NONO; NOTCH1; NOTCH2; NPM1; NR4A3; NRAS; NSD1; NTRK1; NTRK3; NUMA1; NUP214; NUP98; OLIG2; OMD; PAFAH1B2; PALB2; PATZ1; PAX3; PAX5; PAX7; PAX8; PBRM1; PBX1; PCM1; PDE4DIP; PDGFB; PDGFRA; PDGFRB; PER1; PHOX2B; PICALM; PIK3CA; PIK3R1; PIM1; PLAG1; PML; PMS1; PMS2; POU2AF1; POU5F1; PPARG; PPP2R1A; PRCC; PRDM16; PRF1; PRKAR1A; PRRX1; PSIP1; PTCH1; PTEN; PTPN11; RABEP1; RAD50; RAD51L1; RAF1; RANBP17; RAP1GDS1; RARA; RBI; RBM15; RECQL4; REL; RET; RHOH; RNF213; ROS1; RPN1; RPS6KA2; RUNX1; RUNX1T1; SBDS; SDHAF2; SDHB; SETD2; SFPQ; SFRS3; SH3GL1; SLC45A3; SMAD4; SMARCA4; SMARCB1; SMO; SOCS1; SRC; SRGAP3; SS18; SS18L1; STIL; STK11; STK36; SUFU; SYK; TAF15; TAF1L; TAL1; TAL2; TCF12; TCF3; TCL1A; TET1; TET2; TEX14; TFE3; TFEB; TFG; TFRC; THRAP3; TLX1; TLX3; TMPRSS2; TNFAIP3; TOP1; TP53; TPM3; TPM4; TPR; TRIM27; TRIM33; TRIP11; TSC1; TSC2; TSHR; USP6; VHL; WAS; WHSC1L1; WRN; WT1; XPA; XPC; ZBTB16; ZMYM2; ZNF331; ZNF384; and ZNF521.

In some embodiments, the composition includes a plurality of target polynucleotides derived from RNA having at least one mutation associated with cancer, where the mutation associated with cancer is located in at least one of the genes selected from: ABL1; AKT1; ALK; APC; ATM; BRAF; CDH1; CDKN2A; CSF1R; CTNNB1; EGFR; ERBB2; ERBB4; FBXW7; FGFR1; FGFR2; FGFR3; FLT3; GNAS; HNF1A; HRAS; IDH1; JAK2; JAK3; KDR; KIT; KRAS; MET; MLH1; MPL; NOTCH1; NPM1; NRAS; PDGFRA; PIK3CA; PTEN; PTPN11; RB1; RET; SMAD4; SMARCB1; SMO; SRC; STK11; TP53; and VHL.

In some embodiments, the disclosure relates generally to methods, compositions, systems, apparatuses and kits comprising one or more primers. In some embodiments, the primers can be used in a reverse transcription reaction, or a multiplex nucleic acid amplification reaction. In some embodiments, the primers comprise single- or double-stranded DNA, RNA or DNA/RNA hybrid oligonucleotides.

In some embodiments, a primer comprises an oligonucleotide, or a self-priming nucleic acid molecule, having a nucleotide sequence that can hybridize to a target nucleic acid, such as a target RNA, cDNA or DNA molecule. A portion or the entire length of a primer can hybridize to a target nucleic acid. In some embodiments, the primers can hybridize to a target nucleic acid molecule by hydrogen bond formation via Watson-Crick or Hoogstein binding to form a duplex nucleic acid structure. In some embodiments, the hybridizing involves complementary base pairing, including standard A-T or C-G base pairing, or optionally other forms of base-pairing interactions.

In some embodiments, a primer includes an extendible 3′—OH group. In some embodiment, a primer can promote nucleotide polymerization by a polymerase enzyme. In some embodiments, a primer includes a 3′ end having a blocking group that inhibits or blocks primer extension. Optionally, the blocking group is removable with a chemical compound, enzyme, heat or electromagnetic energy.

In some embodiments, a primer can be about 4-100 nts in length, or longer. In some embodiments, a primer used to generate cDNA can be about 5-15 bases, or about 15-30 bases, or about 30-45 bases, or about 45-60 bases, or about 60-75 bases in length, or about 75-100 bases in length, or longer.

In some embodiments, a plurality of primers comprise any one or any mixture of target-specific primers, random-sequence primers, homo-polymer primers (e.g., polyA, polyT, polyG or polyC primers), primers labeled with a detectable moiety, non-labeled primers, and/or primers having the 3′ end linked to at least one blocking group that inhibits or blocks primer extension.

In some embodiments, the disclosure relates generally to methods, compositions, systems, apparatuses and kits, comprising a plurality of target-specific primer pairs which include two or more different pairs of target-specific primers.

In some embodiments, the multiplex nucleic acid amplification reaction includes a plurality of target-specific primer pairs that comprise DNA, RNA or DNA/RNA hybrid oligonucleotides. In some embodiments, the plurality of target-specific primer pairs comprises single-stranded or double-stranded primers.

In some embodiments, the plurality of target-specific primer pairs comprises forward and reverse primers. In some embodiments, a pair of target-specific primers includes a forward and a reverse target-specific primer, or two forward target-specific primers, or two reverse target-specific primers.

In some embodiments, each of the primers in the target-specific primer pairs comprises a sequence that can hybridize to at least one portion of a single target polynucleotide sequence, or comprises a sequence that can hybridize to a complementary sequence of at least one portion of a single target polynucleotide sequence.

In some embodiments, a single pair of target-specific primers hybridizes to any given target polynucleotide. In some embodiments, a single pair of target-specific primers can hybridize to a single target polynucleotide. In some embodiments, a single pair of target-specific primers can hybridize to a single target sequence of cDNA, DNA or RNA. In some embodiments, a single pair of target-specific primers can be used to amplify a single target polynucleotide. In some embodiments, a single pair of target-specific primers can be used to amplify a single target sequence of cDNA, DNA or RNA. In some embodiments, more than one pair of target-specific primers can be used to amplify one cDNA.

In some embodiments, at least one of the target-specific primer pairs has minimal cross-hybridization with any other pair of primers in the single reaction mixture.

In some embodiments, each primer pair in the plurality of target-specific primer pairs is designed to hybridize to a different target polynucleotide sequence of interest. For example, if there are N different target polynucleotides sequences of interest, then the plurality of target-specific primer pairs will contain N different primer pairs. In some embodiments, the plurality of target-specific primer pairs includes 2-100, or about 100-500, or about 500-1,000, or about 1,000-5,000, or about 5,000-10,000, or about 10,000-15,000, or about 15,000-20,000, or about 20,000-25,000, or about 25,000-50,000 or about 50,000-100,000, or more different target-specific primer pairs. In some embodiments, the plurality of target-specific primer pairs includes about 20,000 different target-specific primer pairs.

In some embodiments, a target-specific primer can be about 5-15 bases, or about 15-30 bases, or about 30-45 bases, or about 45-60 bases, or about 60-75 bases in length, or about 75-100 bases in length, or longer.

In some embodiments, the two primers in a target-specific primer pair can be the same length or different lengths.

In some embodiments, at least the 3′ region of a target-specific primer can hybridize to a region of a cDNA molecule. In some embodiments, the entire length of a target-specific primer can hybridize to a region of a cDNA molecule.

In some embodiments, at least one primer in a plurality of target specific primer pairs comprises a tailed primer. In some embodiments, the tailed primer includes a 3′-region having a sequence that hybridizes to at least a portion of a target polynucleotide, and a 5′-region that is a non-hybridizing sequence. In some embodiments, the non-hybridizing portion of a tailed primer includes non-random sequence. In some embodiments, the non-hybridizing portion of a tailed primer can be about 2-10, or about 10-20, or about 20-30, or about 30-50 nucleotides in length.

In some embodiments, plurality of target-specific primer pairs comprises any one or any combination of: labeled primers, non-labeled primers, and/or non-extendible primers. Optionally, the non-extendible primers include a 3′ end linked to at least one blocking group that inhibits or blocks primer extension by a polymerase.

In some embodiments, at least one target-specific primer in a pair of primers can hybridize to an exon sequence, intron sequence, exon/intron junction sequence, or intron/exon junction sequence. In some embodiments, each pair of the target-specific primers can hybridize to a different exon sequence in a different target polynucleotide. In some embodiments, at least one of the primers of the plurality of different target-specific primer pairs can hybridize to a different exon sequence in a different target polynucleotide.

In some embodiments, a primer in a plurality of target-specific primer pairs has minimal cross-hybridization with any other primer in the plurality.

In some embodiments, at least one primer in a plurality of target-specific primer pairs comprises a nucleic acid sequence that is substantially non-complementary to one or more primers in the plurality.

In some embodiments, at least one primer in a plurality of target-specific primer pairs comprises a nucleic acid sequence that is substantially non-self-complementary.

In some embodiments, at least one primer of the plurality of target-specific primer pairs includes at least one cleavable group. Optionally, the cleavable group comprises: uracil, uridine, inosine, or 7,8-dihydro-8-oxoguanine (8-oxoG) nucleobases. Optionally, the cleavable group is cleavable with uracil DNA glycosylase (UDG, also referred to as UNG), formamidopyrimidine DNA glycosylase (Fpg), or a FuPa reagent.

In some embodiments, at least one primer of the plurality of target-specific primer pairs includes or lacks a protecting group that inhibits nucleic acid degradation or digestion.

In some embodiments, at least one of the target-specific primer in the pair of primers includes a unique identifier sequence (e.g., a barcode sequence).

Generally, target-specific primers are designed to minimize the formation of primer-dimers, dimer-dimers or other non-specific amplification products. Typically, target-specific primers are optimized to reduce GC bias and low melting temperatures (Tₘ) during the amplification reaction. In some embodiments, the target-specific primers are designed to possess a Tₘ of about 55° C. to about 72° C. In some embodiments, the target-specific primers of a target-specific primer pool can possess a Tₘ of about 59° C. to about 70° C., 60° C. to about 68° C., or 60° C. to about 65° C. In some embodiments, the target-specific primer pool can possess a Tₘ that does not deviate by more than 5° C. across the target-specific primer pool.

In some embodiments, target-specific primers can be designed de novo using algorithms that generate oligonucleotide sequences according to specified design criteria. For example, the primers may be selected according to any one or more of the criteria specified herein. In some embodiments, one or more of the target-specific primers are selected or designed to satisfy any one or more of the following criteria: (1) inclusion of two or more modified nucleotides within the primer sequence, at least one of which is included near the 3′ end or 5′ end of the target-specific primer and at least one modified nucleotides is included at, or about the center nucleotide position of the target-specific primer sequence; (2) target-specific primer length of about 15 to about 50 bases in length; (3) Tₘ of from about 60° C. to about 70° C.; (4) low cross-reactivity with non-target polynucleotides present in the sample of interest; (5) for each target-specific primer in a given reaction, the sequence of at least the first four nucleotides (going from 3′ to 5′ direction) are not complementary to any sequence within any other target-specific primer present in the same reaction; and (6) no target amplicon includes a consecutive stretch of at least 5 nucleotides that are complementary to another nucleic acid sequence within any other target amplicon.

In some embodiments, the target-specific primers include one or more target-specific primer pairs that amplify target polynucleotides from the sample that are about 100 base pairs to about 1,000 base pairs in length. In some embodiments, the target-specific primers include a plurality of target-specific primer pairs designed to amplify target polynucleotides, where the amplified target polynucleotides vary in length from each other by no more than 50%, typically no more than 25%, 10%, or 5%. For example, if one target-specific primer pair is selected (or predicted) to amplify a product that is 100 nucleotides in length, then other primer pairs are selected (or predicted) to amplify products that are between 50-150 nucleotides in length, typically between 75-125 nucleotides in length, 90-110 nucleotides, 95-105 nucleotides, or 99-101 nucleotides in length.

In one embodiment, at least one primer pair in the amplification reaction is not designed de novo according to any predetermined selection criteria. For example, at least one primer pair can be an oligonucleotide sequence selected or generated at random, or previously selected or generated for other applications. In one exemplary embodiment, the amplification reaction can include at least one primer pair selected from the TaqMan® probe reagents (Roche Molecular Systems). The TaqMan® reagents include labeled probes and can be useful, inter alia, for measuring the amount of target sequence present in the sample, optionally in real time. Some examples of TaqMan technology are disclosed in U.S. Pat. Nos. 5,210,015, 5,487,972, 5,804,375, 6,214,979, 7,141,377 and 7,445,900, hereby incorporated by reference in their entireties.

According to an exemplary embodiment, there is provided a method, comprising: (1) receiving one or more genomic regions or nucleic acid sequences of interest; (2) determining one or more target polynucleotides for the received one or more genomic regions or nucleic acid sequences of interest; (3) providing one or more target-specific primer pairs for each of the determined one or more target polynucleotides; (4) scoring the one or more target-specific primer pairs, wherein the scoring comprises a penalty based on the performance of in silico PCR for the one or more target-specific primer pairs, and optionally, wherein the scoring further comprises an analysis of SNP overlap for the one or more target-specific primer pairs; and (5) filtering the one or more target-specific primer pairs based on a plurality of factors, including at least the penalty and optionally, the analysis of SNP overlap, to identify a filtered set of target-specific primer pairs corresponding to one or more candidate amplicon sequences for the one or more genomic regions or nucleic acid sequences of interest.

In various embodiments, receiving one or more genomic regions or nucleic acid sequences of interest may comprise receiving a list of one or more gene symbols, RNA transcripts or identifiers. Receiving one or more genomic regions or nucleic acid sequences of interest may comprise receiving a list of one or more genomic coordinates or other genomic or transcriptome location identifiers.

In various embodiments, the performance of in silico PCR may comprise performing in silico PCR against a reference or previously sequenced genome or RNA transcriptome, of any species. The performance of in silico PCR may comprise performing in silico PCR against an hg19 reference genome. The performance of in silico PCR against a reference genome or RNA transcriptome may comprise determining a number of off-target hybridizations for each of the one or more target-specific primer pairs. The performance of in silico PCR against a reference genome or RNA transcriptome may comprise determining a worst case attribute or score for each of the one or more target-specific primer pairs. The performance of in silico PCR may comprise determining one or more genomic coordinates or transcriptome identifies for each of the one or more target-specific primer pairs. The performance of in silico PCR may comprise determining one or more predicted amplicon sequences for each of the one or more target-specific primer pairs. The performance of in silico PCR may comprise querying an amplicon or other genomic or transcription sequence database for a presence therein of the one or more genomic regions or nucleic acid sequences of interest or of in silico PCR results for the one or more target specific primer pairs and information related thereto.

In some embodiments, at least one of the target-specific primer pairs within the amplification reaction can be labeled, for example with an optically detectable label, to facilitate a particular application of interest. For example, labeling may facilitate quantification of target polynucleotide and/or amplification product, isolation of the target polynucleotide and/or amplification product, and the like.

In some embodiments, the disclosure generally relates to compositions (as well as related kits, methods, systems and apparatuses using the disclosed compositions) for performing nucleic acid amplification and nucleic acid synthesis. In some embodiments, the compositions include a target-specific primer of about 15 to about 40 nucleotides in length having a uracil nucleotide located near the 3′ or 5′ end of the target-specific primer and a second uracil nucleotide located near a central nucleotide position of the target-specific primer. In some embodiments, the compositions include a target-specific primer of about 15 to about 40 nucleotides in length having an inosine nucleotide located near the 3′ end of the target-specific primer and at least a second inosine nucleotide located near a central nucleotide position of the target-specific primer.

In some embodiments, the disclosure generally relates to compositions (as well as related kits, methods, systems and apparatuses using the disclosed compositions) for performing nucleic acid amplification and nucleic acid synthesis. In some embodiments, one or more of the compositions disclosed herein (as well as related methods, kits, systems and apparatuses) can include at least one target-specific primer and/or at least one adapter. In some embodiments, the compositions include a plurality of target-specific primers (or primer pairs) and adapters that are about 15 to about 40 nucleotides in length. In some embodiments, the compositions include one or more target-specific primers (or primer pairs) or adapters that include one or more cleavable groups. In some embodiments, one or more types of cleavable groups can be incorporated into a target-specific primer (or one or more primer pairs) or an adapter. In some embodiments, a cleavable group can be located at, or near, the 3′ end of a target-specific primer or adapter. In some embodiments, a cleavable group can be located at a terminal nucleotide, a penultimate nucleotide, or any location that corresponds to less than 50% of the total nucleotide length of the target-specific primer or adapter. In some embodiments, a cleavable group can be incorporated at, or near, the central nucleotide of the target-specific primer or the adapter. For example, a target specific primer of 40 bases can include a cleavage group at nucleotide positions 15-25. Accordingly, a target-specific primer or an adapter can include a plurality of cleavable groups within its 3′ end, its 5′ end, or about the central nucleotide position. In some embodiments, the 5′ end of a target-specific primer includes only non-cleavable nucleotides. For example, blocked nucleotides or can be reversibly blocked.

In some embodiments, the cleavable group can include a modified nucleobase or modified nucleotide. In some embodiments, the cleavable group can include a nucleotide or nucleobase that is not naturally occurring in the corresponding nucleic acid. For example, a DNA nucleic acid can include a RNA nucleotide or nucleobase. In one example, a DNA based nucleic acid can include uracil or uridine. In another example, a DNA based nucleic acid can include inosine. In some embodiments, the cleavable group can include a moiety that can be cleaved from the target-specific primer or adapter by enzymatic, chemical or thermal means. In some embodiments, a uracil or uridine moiety can be cleaved from a target-specific primer or adapter using a uracil DNA glycosylase. In some embodiments, an inosine moiety can be cleaved from a target-specific primer or adapter using hAAG or EndoV.

In some embodiments, the disclosure relates generally to methods, compositions, systems, apparatuses and kits, comprise nucleic acid hybridization, include hybridizing primers to RNA, cDNA or target polynucleotides. In some embodiments, any of the reverse transcription or multiplex PCR reactions, according to the present teachings, can include conditions that are suitable for hybridizing primers to nucleic acids. In some embodiments, a plurality of random sequence primers can be hybridized to a plurality of RNA under suitable hybridization conditions to form a primer/RNA complex. In some embodiments, a plurality of target-specific primer pairs can be hybridized to a plurality of target polynucleotides under suitable hybridization conditions to form a primer/polynucleotide complex.

In some embodiments, hybridizing involves hydrogen bond formation via Watson-Crick or Hoogstein binding to form a duplex nucleic acid structure. In some embodiments, the hybridizing involves complementary base pairing, including standard A-T or C-G base pairing, or optionally other forms of base-pairing interactions.

In some embodiments, the suitable hybridizing conditions include hybridizing primers to nucleic acids at a temperature that is close to a calculated or empirically-derived melting temperature. Methods for nucleic acid hybridization are well known in the art. Typically, a thermal melting temperature is calculated for the primers, target template and product. For example, thermal melting temperature (Tₘ) for nucleic acids can be a temperature at which half of the nucleic acid strands are double-stranded and half are single-stranded under a defined condition. In some embodiments, a defined condition can include ionic strength and pH in an aqueous reaction condition. A defined condition can be modulated by altering the concentration of salts (e.g., sodium), magnesium, temperature, pH, buffers, and/or formamide. Typically, the calculated thermal melting temperature can be at about 5-30° C. below the Tₘ, or about 5-25° C. below the Tₘ, or about 5-20° C. below the Tₘ, or about 5-15° C. below the Tₘ, or about 5-10° C. below the Tₘ. Methods for calculating a Tₘ are well known and can be found in Sambrook (1989 in “Molecular Cloning: A Laboratory Manual”, 2^{nd} edition, volumes 1-3; Wetmur 1966, J. Mol. Biol., 31:349-370; Wetmur 1991 Critical Reviews in Biochemistry and Molecular Biology, 26:227-259). Other sources for calculating a Tₘ for hybridizing or denaturing nucleic acids include OligoAnalyze (from Integrated DNA Technologies) and Primer3 (distributed by the Whitehead Institute for Biomedical Research).

In some embodiments, the disclosure relates generally to compositions, as well as related, systems, methods, kits and apparatuses, comprising one or more nucleotides. In some embodiments, the compositions (and related methods, systems, kits and apparatuses) includes one type, or a mixture of different types of nucleotides. A nucleotide comprises any compound that can bind selectively to, or can be polymerized by, a polymerase. Typically, but not necessarily, selective binding of the nucleotide to the polymerase is followed by polymerization of the nucleotide into a nucleic acid strand by the polymerase. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties. In some embodiments, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In some embodiments, one or more phosphorus atoms in the chain can be part of a phosphate group having P and O. In some embodiments, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In some embodiments, the phosphorus atoms in the chain can have side groups having O, BH₃, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281 which is hereby incorporated by reference in its entirety.

Some examples of nucleotides that can be used in the disclosed compositions (and related methods, systems, kits and apparatuses) include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. In some embodiments, a nucleotide can include a purine or pyrimidine base, including adenine, guanine, cytosine, thymine or uracil. In some embodiments, a nucleotide includes dATP, dGTP, dCTP, dTTP and dUTP.

In some embodiments, the nucleotide is unlabeled. In some embodiments, the nucleotide comprises a label and referred to herein as a “labeled nucleotide”. In some embodiments, the label can be in the form of a fluorescent dye attached to any portion of a nucleotide including a base, sugar or any intervening phosphate group or a terminal phosphate group, i.e., the phosphate group most distal from the sugar.

In some embodiments, the disclosure relates generally to compositions, as well as related, systems, methods, kits and apparatuses, comprising any one or any combination of capture primers, reverse primers, fusion primers, target nucleic acids and/or nucleotides that are non-labeled or attached to at least one label. In some embodiments, the label comprises a detectable moiety. In some embodiments, the label can generate, or cause to generate, a detectable signal. In some embodiments, the detectable signal can be generated from a chemical or physical change (e.g., heat, light, electrical, pH, salt concentration, enzymatic activity, or proximity events). For example, a proximity event can include two reporter moieties approaching each other, or associating with each other, or binding each other. In some embodiments, the detectable signal can be detected optically, electrically, chemically, enzymatically, thermally, or via mass spectroscopy or Raman spectroscopy. In some embodiments, the label can include compounds that are luminescent, photoluminescent, electroluminescent, bioluminescent, chemiluminescent, fluorescent, phosphorescent or electrochemical. In some embodiments, the label can include compounds that are fluorophores, chromophores, radioisotopes, haptens, affinity tags, atoms or enzymes. In some embodiments, the label comprises a moiety not typically present in naturally occurring nucleotides. For example, the label can include fluorescent, luminescent or radioactive moieties.

In some embodiments, the disclosure relates generally to compositions, as well as related, systems, methods, kits and apparatuses, comprising cleaved amplified nucleic acids linked to one or more adaptors to generate adapter-ligated amplified nucleic acids.

In some embodiments, one or more adaptors can be joined to the cleaved amplified nucleic acid by ligation. In some embodiments, a tailed amplification primer can be used in a PCR reaction to append one or more adaptors to an amplicon or a cleaved amplified nucleic acid, where the tailed amplification primer includes the sequence of one or more adaptors.

In some embodiments, the adaptor comprises a nucleic acid, including DNA, RNA, RNA/DNA molecules, or analogs thereof. In some embodiments, the adaptor can include one or more deoxyribonucleoside or ribonucleoside residues. In some embodiments, the adaptor can be single-stranded or double-stranded nucleic acids, or can include single-stranded and/or double-stranded portions. In some embodiments, the adaptor can have any structure, including linear, hairpin, forked (Y-shaped), or stem-loop (see U.S. Pat. Nos. 7,741,463; 8,029,993; 8,053,192; 8,182,989; 8,563,478; 8,822,150, which are incorporated by reference in their entirety).

In some embodiments, the adaptor can have any length, including fewer than 10 bases in length, or about 10-20 bases in length, or about 20-50 bases in length, or about 50-100 bases in length, or longer.

In some embodiments, the adaptor can have any combination of blunt end(s) and/or sticky end(s). In some embodiments, at least one end of the adaptor can be compatible with at least one end of a cleaved amplified nucleic acid. In some embodiments, a compatible end of the adaptor can be joined to a compatible end of an amplified nucleic acid. In some embodiments, the adaptor can have a 5′ or 3′ overhang end.

In some embodiments, the adaptor can have a 5′ or 3′ overhang tail. In some embodiments, the tail can be any length, including 1-50 or more nucleotides in length. In some embodiments, the adapter overhang includes a homopolymeric stretch of at least 5, 10, 20 or 25 identical contiguous nucleotide residues.

In some embodiments, the adaptor can include an internal nick. In some embodiments, the adaptor can have at least one strand that lacks a terminal 5′ phosphate residue. In some embodiments, the adaptor lacking a terminal 5′ phosphate residue can be joined to a cleaved amplified nucleic acid to introduce a nick at the junction between the adaptor and the cleaved amplified nucleic acid.

In some embodiments, the adaptor can include a nucleotide sequence that is identical or complementary to any portion of a capture primer, fusion primer, reverse primer, amplification primer, or a sequencing primer.

In some embodiments, the adaptor can include identification sequences, such as for example, a uniquely identifiable sequence (e.g., barcode sequence). In some embodiments, a barcoded adaptor can be used for constructing a multiplex library of amplified nucleic acids. In some embodiments, the barcoded adaptors can be appended to a cleaved amplified nucleic acid and used for sorting or tracking the source of the target polynucleotide. In some embodiments, one or more barcode sequences can allow identification of a particular adaptor among a mixture of different adaptors having different barcodes sequences. For example, a mixture can include 2, 3, 4, 5, 6, 7-10, 10-50, 50-100, 100-200, 200-500, 500-1000, or more different adaptors having unique barcode sequences.

In some embodiments, the adaptor can include degenerate sequences. Optionally, different adaptors can include different degenerate sequences. In some embodiments, the adaptor can include one or more inosine residues. For example, the stem portion of a Y-shaped or stem-loop adaptor can include at least one degenerate sequence, or at least one inosine residue.

In some embodiments, the adaptor can include at least one scissile linkage. In some embodiments, the scissile linkage can be susceptible to cleavage or degradation by an enzyme or chemical compound. In some embodiments, the adaptor can include at least one phosphorothiolate, phosphorothioate, and/or phosphoramidate linkage.

In some embodiments, the adaptor can include any type of restriction enzyme recognition sequence, including type I, type II, type Hs, type IIB, type III, type IV restriction enzyme recognition sequences, or recognition sequences having palindromic or non-palindromic recognition sequences.

In some embodiments, the adaptor can include a cell regulation sequences, including a promoter (inducible or constitutive), enhancers, transcription or translation initiation sequence, transcription or translation termination sequence, secretion signals, Kozak sequence, cellular protein binding sequence, and the like.

In some embodiments, the disclosure relates generally to compositions, and related methods, systems, kits and apparatuses, comprising mutant sequences. In some embodiments, any target polynucleotide, amplicon or adaptor-ligated amplified nucleic acid can include a mutant sequence (e.g., aberrant sequence). In some embodiments, the mutant sequence includes any sequence that differs from a wild-type or normal sequence. In some embodiments, the mutant sequence includes any one or any combination of nucleotide: deletions, insertions, or substitutions or one or more nucleotides; inversions; rearrangements; truncations; and/or variant or abnormal splice junction sequences.

In some embodiments, the reverse transcription or the multiplex nucleic acid amplification step can include a nucleic acid digestion step. The digestion step can be conducted before or after any step of the disclosed methods. The digestion step can be conducted enzymatically, chemically, with light or with heat.

In some embodiments, the disclosure relates generally to compositions, and related methods, systems, kits and apparatuses, comprising a reverse transcription reaction, or nucleic acid amplification reaction that can be conducted under thermocycling or isothermal conditions, or a combination of both types of conditions.

In some embodiments a reaction mixture for conducting a reverse transcription reaction or a nucleic acid amplification reaction that is subjected to a temperature variation which is constrained within a limited range during at least some portion of the reverse transcription or amplification, including for example a temperature variation is within about 20° C., or about 10° C., or about 5° C., or about 1-5° C., or about 0.1-1° C., or less than about 0.1° C.

In some embodiments, an isothermal nucleic acid amplification reaction can be conducted for about 2, 5, 10, 15, 20, 30, 40, 50, 60 or 120 minutes, or longer.

In some embodiments, an isothermal nucleic acid amplification reaction can be conducted at about 15-30° C., or about 30-45° C., or about 45-60° C., or about 60-75° C., or about 75-90° C., or about 90-93° C., or about 93-99° C.

In some embodiments, the multiplex amplification reactions is conducted under temperature-cycling conditions (U.S. Pat. Nos. 4,683,202, 4,683,195, 4,889,818, hereby incorporated by reference in their entireties).

In some embodiments, the disclosure relates generally to methods, compositions, systems, apparatuses and kits, comprising a reaction vessel which includes a tube (e.g., Eppendorf™ tube), inner wall of a tube, well, reaction chamber, groove, channel reservoir, or flowcell.

In some embodiments, two or more reaction vessels can be two or more reaction chambers arranged in an array. In some embodiments, the array can include one or more reaction chambers on a solid support. A reaction chamber can have walls that define width and depth. The dimensions of a reaction chamber can be sufficient to permit deposition of reagents or for conducting reactions. A reaction chamber can have any shape including cylindrical, polygonal or a combination of different shapes. Any wall of a reaction chamber can have a smooth or irregular surface. A reaction chamber can have a bottom with a planar, concave or convex surface. The bottom and side walls of a reaction chamber can comprise the same or different material and/or can be coated with a chemical group that can react with a biomolecule such as nucleic acids, proteins or enzymes.

In some embodiments, the reaction chamber can be one of multiple reaction chambers arranged in a grid or array. An array can include two or more reaction chambers. Multiple reaction chambers can be arranged randomly or in an ordered array. An ordered array can include reaction chambers arranged in a row, or in a two-dimensional grid with rows and columns.

An array can include any number of reaction chambers for depositing reagents and conducting numerous individual reactions. For example, an array can include at least 256 reaction chambers, or at least 256,000, or at least 1-3 million, or at least 3-5 million, or at least 5-7 million, or at least 7-9 million, at least 9-11 million, at least 11-13 million reaction chambers, or even high density including 13-700 million reaction chambers or more. Reaction chambers arranged in a grid can have a center-to-center distance between adjacent reaction chambers (e.g., pitch) of less than about 10 microns, or less than about 5 microns, or less than about 1 microns, or less than about 0.5 microns.

An array can include reaction chambers having any width and depth dimensions. For example, a reaction chamber can have dimensions to accommodate a single microparticle (e.g., microbead) or multiple microparticles. A reaction chamber can hold 0.001-100 picoliters of aqueous volume.

In some embodiments, at least one reaction vessel (e.g., at least one reaction chamber) can be coupled to one or more sensors or can be fabricated above one or more sensors. A reaction chamber that is coupled to a sensor can provide confinement of reagents deposited therein so that products from a reaction can be detected by the sensor. A sensor can detect changes in products from any type of reaction, including any nucleic acid reaction such as primer extension, amplification or nucleotide incorporation reactions, within the reaction vessel. A sensor can detect changes in ions (e.g., hydrogen ions), protons, phosphate groups such as pyrophosphate groups. A sensor can detect at least one by product of nucleotide incorporation, including pyrophosphate, hydrogen ions, charge transfer, or heat. In some embodiments, at least one reaction chamber can be coupled to one or more field effect transistor (FET), including for example an ion sensitive field effect transistor (ISFET). Examples of an array of reaction chambers coupled to ISFET sensors can be found at U.S. Pat. No. 7,948,015, and U.S. Ser. No. 12/002,781, hereby incorporated by reference in their entireties. Other examples of sensors that detect byproducts of a nucleotide incorporation reaction can be found, for example, in Pourmand et al, Proc. Natl. Acad. Sci., 103: 6466-6470 (2006); Purushothaman et al., IEEE ISCAS, IV-169-172; Anderson et al, Sensors and Actuators B Chem., 129: 79-86 (2008); Sakata et al., Angew. Chem. 118:2283-2286 (2006); Esfandyapour et al., U.S. Patent Publication No. 2008/01666727; and Sakurai et al., Anal. Chem. 64: 1996-1997 (1992) (which are all hereby incorporated by reference in their entireties).

In some embodiments, any of the methods for characterizing RNA, according to the present teachings, can be conducted manually or by automation. In some embodiments, the steps of amplifying, analyzing, comparing, reverse transcribing, nucleic acid amplification, cleaving, adapter-ligating, characterizing, and/or sequencing, can be conducted manually or by automation. For example, any reagents for conducting any of these steps can be deposited into, or removed from, a reaction vessel via manual or automated modes.

In some embodiments, the methods of the disclosure can be performed as “addition-only” processes. In some embodiments, the “addition-only” process excludes the removal of all, or a portion of the first reaction mixture including the amplifying compositions, for further manipulation during the amplification steps, ligation and/or digestion steps. In some embodiments, the “addition-only” process can be automated for example for use in high-throughput processing.

In some embodiments, the disclosed methods, compositions, systems, apparatuses and kits for characterizing RNA or DNA offers advantages over conventional methods. For example, unlike other transcriptome analysis methods that require a starting sample that contains polyA RNA, one embodiment of the disclosed methods, compositions, systems, apparatuses and kits employs random-sequence primers in the reverse transcription step. The random-sequence primers are designed to hybridize to many different types of RNA, including polyA and non-polyA RNA, which permits analysis of total RNA samples. Use of the random-sequence primers also obviates the requirement for a priori knowledge of the RNA sequences. Additionally, conducting the reverse transcription step with random-sequence primers generates a population of cDNA with improved representation of the original RNA population present in the starting sample. Preparing RNA samples having reduced sequence representation bias is important for RNA abundance analyses. Additionally, conducting the reverse transcription step with random-sequence primers reduces 3′ sequence bias, which is prevalent when using polyT primers for priming polyA RNA.

In some embodiments, the disclosed methods, compositions, systems, apparatuses and kits can be used to characterize RNA or DNA from any type of sample, including those from fresh or archived samples, or total RNA or pre-enriched RNA samples. Although the methods do not require pre-enrichment procedures, the results can be optimized by removal of rRNA, or other abundant RNA species, using procedures such as RNA depletion, polyA selection, size selection, size modification, or RNA-protein complex selection.

In some embodiments, the disclosed methods, compositions, systems, apparatuses and kits for characterizing RNA or DNA can be conducted in a single reaction vessel, which eliminates centrifugation steps, and transfer of the reagents to a fresh tube. This simplified the workflow requires fewer steps that would cause loss of nucleic acid material, and enables amplification of a sequence-of-interest from a sample containing as little as 500 pg of RNA (unfixed samples) or 5 ng RNA from FFPE samples.

In some embodiments, the disclosed methods, compositions, systems, apparatuses and kits for characterizing RNA or DNA includes a multiplex amplification reaction performed in a single reaction mixture using hundreds, thousands, tens-of-thousands or even hundreds of thousands of different target-specific primer pairs that enable substantially simultaneous amplification of many-thousands of different target polynucleotide sequences-of-interest, to more accurately reflect the complexity and abundance of the RNA or DNA sequences of interest in the sample. This ultra-plexy amplification reaction eliminates the requirement to perform separate amplification reactions and pooling, which simplifies the workflow, and reduces variations in amplification efficiency that arise in separate reaction vessels.

In some embodiments, the disclosed methods, compositions, systems, apparatuses and kits for characterizing RNA or DNA includes a multiplex amplification reaction, where each target-specific primer pair is designed to hybridize to a single target polynucleotide sequence of interest. In some embodiments, the multiplex amplification reactions of the present teachings can yield data that more accurately measures transcript abundance because each target-specific primer pair has approximately a one-to-one correspondence with a single target polynucleotide sequence. Thus, the number of amplicons containing the same (or substantially the same) sequence that are formed in the multiplex amplification step more directly reflects the abundance of a sequence-of-interest from which the amplicons were derived. In some embodiments, the number of amplicons identified as containing a first target sequence of interest can be determined to obtain a first number. In some embodiments, the first number can be used to calculate a first abundance value for the first target sequence. Optionally, the number of amplicons identified as containing a second target sequence of interest can also be determined to obtain a second number, optionally in the same sequencing assay or in a different and separate sequencing assay. In some embodiments, the methods can include determining a second abundance value for the second target sequence using the second number. The relative abundances of the first and second target sequences can be compared, optionally as a ratio of the first and second numbers, or as a percentage (e.g., percentage of total sequence reads containing the first and/or second target sequence), or using any other suitable calculation method.

In some embodiments, the disclosure relates generally to methods, compositions, systems, apparatuses and kits for detecting a plurality of polynucleotides in a sample, comprising: a method for detecting a plurality of polynucleotides in a sample, comprising: (a) contacting, within a single reaction mixture, a plurality of target-specific primer pairs with a plurality of target polynucleotides derived from the sample, under nucleic acid hybridization conditions such that different target-specific primer pairs hybridize to different target polynucleotides, wherein the different target-specific primer pairs include at least one cleavable group; (b) extending the target-specific primer pairs to form a plurality of amplicons which contain a sequence derived from a target polynucleotide and a primer-derived sequence which includes the at least one cleavable group; (c) cleaving the at least one cleavable group of the plurality of amplicons to generate a plurality of cleaved amplicons; and (d) joining one or both ends of the plurality of cleaved amplicons to a Y-shaped adaptor to form a plurality of adaptor-joined amplicons. In some embodiments, the method further comprises: detecting the adaptor-joined amplicons. Optionally, the detecting of step (e) comprises sequencing the plurality of adaptor-joined amplicons. In some embodiments, the method further comprises: re-amplifying the plurality of adaptor-joined amplicons. In some embodiments, the method further comprises: attaching the adaptor-joined amplicons to a support or to a plurality of supports. In some embodiment, the support includes a plurality of first and second capture primers attached thereon, and the plurality of first and second capture primers have different sequences. In some embodiments, the adaptor-joined amplicons are attached to the support by: (i) rendering the adaptor-joined amplicons single-stranded to generate single-stranded adaptor-joined molecules; (ii) hybridizing the single-stranded adaptor-joined molecules to the plurality of first capture primers; and (iii) extending the plurality of first capture primers to generate a plurality of first capture primer extension products. Optionally, the plurality of supports includes a plurality beads with a plurality of capture primers attached thereon. In some embodiments, the adaptor-joined amplicons can be attached to the support by: conducting a recombinase polymerase amplification (RPA) reaction under an isothermal amplification condition with: (i) a polymerase; (ii) a plurality of nucleotides; (iii) a recombinase; (iv) a recombinase loading factor; (v) a single-stranded binding protein; and (vi) a plurality of soluble reverse primers. In some embodiments, the sequencing can be conducted by polymerase-mediated incorporation of a terminator nucleotide which is blocked at the 2′ or 3′ OH sugar position of the base. Optionally, the terminator nucleotide can be attached with an optically-detectable dye. In some embodiments, the sequencing of step (e) can be conducted by polymerase-mediated incorporation of a non-labeled and non-blocked nucleotide. In some embodiments, the sequencing of step (e) can be conducted by detecting changes in release of protons, hydrogen ions, charge transfer or heat. In some embodiments, the plurality of target-specific primer pairs includes 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target-specific primer pairs. In some embodiments, the extending in step (b) includes forming a plurality of amplicons containing sequences derived from 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target polynucleotides. In some embodiments, the detecting the adaptor-joined amplicons comprises: quantifying the amount of adaptor-joined amplicons containing sequence derived from the 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target polynucleotides. In some embodiments, the sample comprises DNA, RNA or cDNA. In some embodiments, the sample includes cell-free DNA or cell-free RNA. In some embodiments, the sample is derived from a single cell or a population of cells. In some embodiments, the sample is derived from prokaryotes, eukaryotes, fungus or virus. In some embodiments, the sample is derived from a bodily fluid selected from a group consisting of blood, urine, serum, lymph, tumor, saliva, anal and vaginal secretions, amniotic samples, perspiration, and semen.

In some embodiments, the disclosure relates generally to methods, compositions, systems, apparatuses and kits for detecting a plurality of polynucleotides in a sample, comprising: a method for detecting a plurality of polynucleotides in a sample, comprising: (a) contacting, within a single reaction mixture, a plurality of different target-specific primer pairs with a plurality of target polynucleotides derived from the sample, wherein the plurality of target polynucleotides includes at least a first and a second target polynucleotide, under nucleic acid hybridization conditions such that different target-specific primer pairs hybridize to different target polynucleotides, wherein the different target-specific primer pairs include at least one cleavable group; (b) extending the target-specific primer pairs to form a plurality of first amplicons which contain a sequence derived from the first target polynucleotide and a primer-derived sequence which includes the at least one cleavable group, and extending the target-specific primer pairs to form a plurality of second amplicons which contain a sequence derived from the second target polynucleotide and a primer-derived sequence which includes the at least one cleavable group; (b) cleaving the at least one cleavable group of the first and second plurality of amplicons to generate a plurality of first and second cleaved amplicons; (c) joining one or both ends of the plurality of first and second cleaved amplicons to a Y-shaped adaptor to form a plurality of first and second adaptor-joined amplicons; and (d) detecting the first and second adaptor-joined amplicons. In some embodiments, the detecting the first and second adaptor-joined amplicons comprises: determining an amount of amplicons containing a sequence derived from the first target polynucleotide and determining an amount of amplicons containing a sequence derived from the second target polynucleotide. In some embodiments, the detecting the first and second adaptor-joined amplicons comprises: quantifying the amount of the first target polynucleotide in the sample, and quantifying the amount of the second target polynucleotide present in the sample. In some embodiments, the method further comprises: calculating a ratio of the amount of adaptor-joined amplicons derived from the first target polynucleotide, and the amount of adaptor-joined amplicons derived from the second target polynucleotide. Optionally, the detecting of step (d) comprises sequencing the plurality of adaptor-joined amplicons. In some embodiments, the Y-shaped adaptor can be joined to one or both ends of the plurality of cleaved amplicons by enzymatic ligation. Optionally, the Y-shaped adaptor includes a unique identifier sequence or a degenerate sequence. Optionally, the Y-shaped adaptor includes a sequencing primer binding site, an amplification primer binding site or a restriction enzyme recognition sequence. Optionally, the Y-shaped adaptor includes a 5′ or 3′ overhang end. In some embodiments, the different target-specific primer pairs of step (a) are tailed primer pairs or non-tailed primer pairs. In some embodiments, the cleavable group of step (b) is cleavable with an enzyme, chemical compound, heat or light. In some embodiments, the cleavable group of step (b) is cleavable with a uracil DNA glycosylase (UDG) or a formamidopyrimidine DNA glycosylase (Fpg). In some embodiments, the cleavable group of step (b) is cleavable with a FuPa reagent which includes a DNA polymerase, a uracil-cleaving enzyme, and an antibody that inhibits activity of the DNA polymerase. In some embodiments, the at least one cleavable group of step (b) comprises uracil, uridine, inosine, or 7,8-dihydro-8-oxoguanine (8-oxoG) nucleobases. In some embodiments, the plurality of target-specific primer pairs includes 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target-specific primer pairs. In some embodiments, the extending in step (b) includes forming a plurality of amplicons containing sequences derived from 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target polynucleotides. In some embodiments, the detecting the first and second adaptor-joined amplicons comprises: quantifying the amount of adaptor-joined amplicons containing sequence derived from the 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target polynucleotides. In some embodiments, the sample comprises DNA, RNA or cDNA. In some embodiments, the sample includes cell-free DNA or cell-free RNA. In some embodiments, the sample is derived from a single cell or a population of cells. In some embodiments, the sample is derived from prokaryotes, eukaryotes, fungus or virus. In some embodiments, the sample is derived from a bodily fluid selected from a group consisting of blood, urine, serum, lymph, tumor, saliva, anal and vaginal secretions, amniotic samples, perspiration, and semen.

The disclosed methods, compositions, systems, apparatuses and kits for characterizing RNA or DNA includes target-specific primers that enable a streamlined library prep workflow, because they include at least one cleavable group which is used to create nucleic acid fragment ends that are ready for adapter-joining and sequencing.

The disclosed methods, compositions, systems, apparatuses and kits for characterizing RNA or DNA includes substantially reduced sequence read assembly, or sequence read assembly is not performed, because a single pair of target-specific primers is configured to generate a single sequence for each target polynucleotide.

Thus, the disclosed methods, compositions, systems, apparatuses and kits for characterizing RNA offers a one-pot amplification reaction that requires very small amounts of starting RNA, does not require pre-enrichment, yields amplified nucleic acids with improved transcript sequence representation that are sequence-ready with fewer steps.

### EXAMPLES

Embodiments of the present teachings can be further understood in light of the following examples, which should not be construed as limiting the scope of the present teachings in any way.

### Example 1

A tube of diluted ERCC Spike-In Mix was prepared. 9 uL of nuclease-free water was distributed into each of two 0.5 mL tubes. The tubes were labeled 1:10, 1:100 and 1:1,000. One uL of ERCC Spike-In Control Mix was added to the 1:10 tube and mixed by vortexing, and spun down. One uL of the 1:10 diluted ERCC was transferred to the 1:100 tube and mixed by vortexing, and spun down. One uL of the 1:100 diluted ERCC was transferred to the 1:1,000 tube and mixed by vortexing, and spun down.

The RNA used for this experiment was from one of three different sources, including: a commercially-available mixture of RNA from different multiple tissue types and individuals (Universal Human Reference, from Agilent, catalog No. 740000); or a mixture of RNA from multiple individuals (Human Brain Reference, from Ambion, catalog No. AM6050), or RNA extracted from FFPE samples that originated from various tissues and individuals.

The starting concentration of one RNA sample was 100 ng/L. One uL of the 100 ng/uL RNA was mixed with 1 uL of the 1:100 diluted ERCC tube. The final concentration of this RNA was about 50 ng/uL. The starting concentration of another RNA sample was 10 ng/uL. One uL of the 10 ng/uL RNA was mixed with 1 uL of the 1:1,000 diluted ERCC tube. The final concentration of this RNA was about 5 ng/uL. The ERCC Spike-In Control Mix was not added to FFPE RNA samples.

A reverse transcription reaction was set up in a 96-well plate. The VILO RT Reaction Mix and SuperScript III Enzyme Mix were from a SuperScript® VILO™ cDNA Synthesis Kit (Life Technologies, catalog No. 11754-050). A reverse transcription reaction having a total volume of 5 uL contained: 2 uL of RNA (the 5 ng/uL or 50 ng/uL RNA sample), 1 uL of 5×VILO RT Reaction Mix (which contains random-sequence hexamer primers), 0.5 uL of 10× SuperScript III Enzyme Mix, and 1.5 uL of nuclease-free water. The reaction was gently vortexed and spun briefly. The reverse transcription reaction was incubated at 42° C. for 30 minutes, and inactivated at 85° C. for 5 minutes.

The cDNA generated from the RNA sample above was used to generate amplified target nucleic acids having both ends appended with a primer-derived sequence. A PCR amplification reaction mixture was set up in a 0.5 mL or 1.5 mL tube. The 5× Ion AmpliSeq HiFi Master Mix was obtained from an Ion AmpliSeq Library Kit Plus (Life Technologies, catalog No. 448890AB). The total volume of the PCR amplification reaction mixture was 15 uL and contained: 4 uL of 5× Ion AmpliSeq HiFi Master Mix (red cap), 8 uL of 21K Primer Panel, 1 uL of 20×10 ERCC primer panel, and 2 uL of nuclease-free water. The 21K Primer Panel contained roughly 20,000 different pairs of target-specific primers, and each pair was designed to hybridize to one target polynucleotide sequence having an exon sequence using proprietary primer design parameters and algorithms, including those described in further detail in U.S. Pat. No. 8,673,560. The amplification reaction mixture was gently vortexed and spun down. 15 uL of the amplification reaction mixture was added to the reverse transcription reaction in the 96-well plate. The plate was sealed, gently vortexed to mix, and spun down. The plate was loaded into a thermo-cycler and run according to the following conditions.

| Stage: | Temperature: | Time: | |
|---|---|---|---|
| Hold | 99° C. | 2 | min. |
| Cycle: set number | 99° C. | 15 | sec. |
| according to the following | 60° C. | 16 | min. |
| table | | | |

| | | |
|---|---|---|
| Hold | 10° C. | ∞ |

| | |
|---|---|
| Input RNA: | # of cycles |

| | | |
|---|---|---|
| Unfixed RNA | 10 ng | 12 |
| | 100 ng | 10 |
| FFPE RNA | 10 ng | 16 |
| | 100 ng | 13 |

The primer-derived sequences that were appended to the ends of the amplified target nucleic acids were partially digested (cleaved) by adding to the PCR amplification reaction mixture, 2 uL of FuPa Reagent (brown cap) which was obtained from the Ion AmpliSeq Library Kit Plus (Life Technologies, catalog No. 448890AB). The mixture was mixed by pipetting up and down 5 times or by gentle vortexing. The plate was sealed and loaded into a thermo-cycler, and run according to the following conditions.

| Temperature: | Time: |
|---|---|
| 50° C. | 10 min. |
| 55° C. | 10 min. |
| 60° C. | 20 min. |
| 10° C. | Hold (for up to 1 hour) |

Adapters were ligated to the partially digested (cleaved) samples in a ligation reaction. The Switch Solution and DNA ligase were obtained from an Ion AmpliSeq Library Kit Plus (Life Technologies, catalog No. 448890AB).

For ligating non-barcoded adapters, to each well containing the partially digested samples (22 uL), add: 4 uL of Switch solution (yellow cap), 2 uL of Ion AmpliSeq Adaptors (non-barcoded, green cap) or 2 uL of diluted barcode adaptor mix (barcoded adaptors). The plate was sealed, and mixed by gentle vortexing and spun down. To each well, 2 uL of DNA Ligase (blue cap) was added. The plate was re-sealed, and mixed by gentle vortexing and spun down.

For ligating barcoded adaptors, a diluted adaptor mix was prepared by mixing together: 2 uL of Ion P1 adaptor (violet cap), 2 uL of Ion AmpliSeq Barcode X (white cap), and 4 uL nuclease-free water. To each well containing the partially digested samples (22 uL), add: 4 uL of Switch solution (yellow cap), 2 uL of the diluted adaptor mix. The plate was sealed, and mixed by gentle vortexing and spun down. To each well, 2 uL of DNA Ligase (blue cap) was added. The plate was re-sealed, and mixed by gentle vortexing and spun down.

For the non-barcoded and the barcoded ligation reactions, the plate was loaded into a thermo-cycler and run according to the following conditions.

| Temperature: | Time: |
|---|---|
| 22° C. | 30 min. for unfixed RNA or |
| | 60 min. for FFPE RNA |
| 72° C. | 10 min. |
| 10° C. | Hold (up to 1 hour) |

The adaptor-ligated library was purified using Ampure XP beads. For each sample, a mixture was prepared containing 230 uL of freshly prepared 70% ethanol and 100 uL of nuclease-free water. To each sample in the 96-well plate, 45 uL of Ageneourt® AMPure® XP Reagent (1.5× sample volume) was added, and mixed by pipetting up and down five times, and incubated at room temperature for 5 minutes. The plate was placed in a magnetic stand and incubated for 2 minutes, or until the solution turned clear. The supernatant was carefully removed without disturbing the pellet. The beads were washed by adding 150 uL of the freshly prepared ethanol mixture, and the plate was moved side-to-side in the two positions of the magnetic stand. The supernatant was carefully removed without disturbing the pellet. The beads were re-washed using the same wash procedure. The supernatant was carefully removed without disturbing the pellet, and all the ethanol droplets were removed from the wells. The beads were air-dried for about 2 minutes at room temperature. The plate was removed from the magnetic stand. The beads were dispersed by adding 50 uL of Low TE to the pellet to disperse the beads. The plate was sealed, and vortexed thoroughly, and spun down to collect the droplets. The plate was placed on a magnetic stand for at least 2 minutes. About 45 uL of the supernatant was transferred to new wells (e.g., on the same plate).

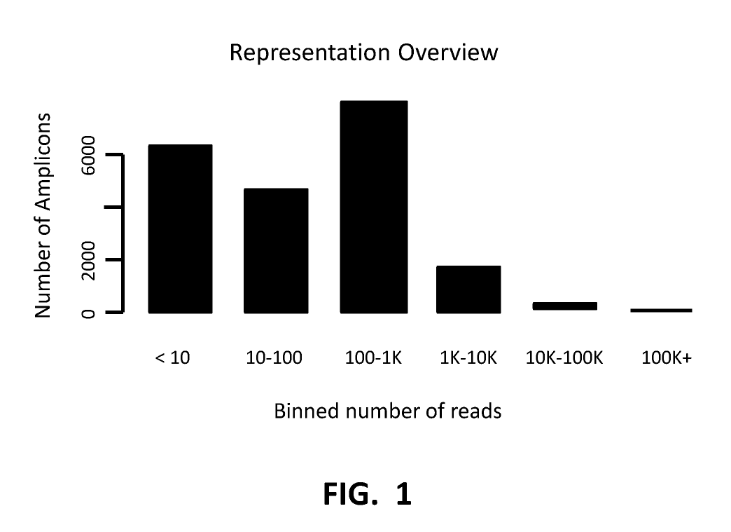

The adaptor-ligated library was quantified. About 1 pM of the library was used in a bead templating workflow using an Ion PI™ Template OT 200 Kit (Life Technologies, catalog No. 4488318) according to the manufacturer's instructions, and the templated beads were used for sequencing on an Ion Torrent™ Proton™ I chip on an Ion Torrent™ Proton™ instrument according to the manufacturer-provided protocols (Ion Proton™ System, catalog No. 4476610, and Ion PI™ Sequencing 200 Kit v3, catalog No. 4488315). The resulting sequence reads from each well (each read corresponding to sequence from one templated bead) were mapped and counted using the Torrent Suite™ browser software and in-house scripts. Representative data showing read counts for different sequence reads for different target polynucleotide sequences derived from the Universal Human Reference or the Human Brain Reference samples is presented in FIG. 1 and TABLES 1 and 2.

The different target polynucleotide sequences contained in the adaptor-ligated library were mapped against a list of different target sequences of interest, where each target sequence of interest correlated with a single target-specific primer pair. The number of reads corresponding to each of the different target polynucleotide sequences was binned and counted. FIG. 1 shows a graph of a tally of the number of different amplicon sequences that yielded less than 10, 10-100, 100-1000, 1000-10,000, or more than 10,000 reads. More than 11 million reads were mapped (TABLE 1), which contained over 20,000 different amplicon sequences. Of the 20,812 different amplicon sequences that were mapped and counted using an AmpliSeq RNA plugin in Torrent Suite (Torrent Suite™ Software, version 4.0.2, user interface guide, document revision November 2013 Rev. A), the most abundant transcript sequences (yielding at least 10,000 reads each) were represented by only 103 different target sequences, and the least abundant transcript sequences (yielding at least one read each) were represented by more than 17,000 different target sequences (TABLE 2). An example of the raw counts of sequencing reads for two different amplicons, from 8 different libraries that were generated from 8 separate amplification reactions, is provided in TABLE 3. The data from TABLE 3 indicates that transcripts having the AARS sequence are approximately 11 times (e.g., 11-fold) more abundant compared to transcripts having the ABCB10 sequence.

TABLE 1

| | |
|---|---|
| Number of mapped reads | 11,267,828 |
| Percent reads on target | 91.40% |
| Percent assigned reads | 89.99% |
| Percent ERCC tracking reads | 0.63% |

TABLE 2

| | |
|---|---|
| Number of amplicons | 20,812 |
| Amplicons with at least 1 read | 17,970 |
| Amplicons with at least 10 reads | 14,556 |
| Amplicons with at least 100 reads | 9.938 |
| Amplicons with at least 1000 reads | 2,017 |
| Amplicons with at least 10,000 reads | 103 |

TABLE 3

| Gene/Target: | AARS/AMPL10784042 | ABCB10/AMPL12686552 |
|---|---|---|
| Library 1 | 3759 | 384 |
| Library 2 | 2914 | 376 |
| Library 3 | 3810 | 466 |
| Library 4 | 3375 | 401 |
| Library 5 | 3938 | 224 |
| Library 6 | 5415 | 329 |
| Library 7 | 5204 | 319 |
| Library 8 | 4536 | 286 |

### Example 2

### Expression Analysis

Six different RNA samples were converted into cDNA and amplified using a pool of roughly 20,000 different target-specific primer pairs (referring to herein as the “Transcriptome Primer Panel”) as described for Example 1 above. The transcriptome primer panel was designed to include one single set of target-specific primers for each of the different transcripts present in a typical human transcriptome. For each RNA sample, the resulting amplicons were adapted via attachment of Ion Torrent standard adapters including one of 6 different barcodes (named “IonXpress_002” through “IonXpress_008”) to generate 6 different transcriptome libraries, each attached to a different identifying barcode. The resulting libraries were pooled, subjected to emulsion PCR according to the Ion PI™ Template OT2 200 Kit v3 (catalog No. 4488318) protocol, and sequenced on the Ion Torrent Proton System, essentially as described for Example 1. Sequencing reads were mapped to a reference genome using proprietary software scripts in conjunction with standard Torrent Suite™ software provided with the Ion Torrent™ Proton™ system. The following numbers of Mapped Reads, On Target Reads and Total Targets were detected (TABLE 4):

TABLE 4

| | | | Targets |
|---|---|---|---|
| Barcode Name | Mapped Reads | On Target | Detected |
| IonXpress_002 | 12,384,502 | 94.22% | 69.98% |
| IonXpress_004 | 12,949,114 | 95.41% | 71.41% |
| IonXpress_005 | 13,311,810 | 94.34% | 70.23% |
| IonXpress_006 | 12,723,572 | 94.32% | 69.94% |
| IonXpress_007 | 14,024,941 | 95.36% | 71.93% |
| IonXpress_008 | 13,222,606 | 95.50% | 71.22% |

The number of sequencing reads mapped to each of the approximately 20,000 different transcripts targeted using the transcriptome primer panel were counted to derive an indication of the abundance of each transcript in the sample. Exemplary read counts for the first 100 transcripts targeted by the transcriptome primer panel are provided in TABLE 4 below (each one of the 6 different libraries, as identified by barcode, referred to in TABLE 4 as “Lib′y #1”, “Lib′y #2”, etc.). In theory, each sequencing read provides the sequence a single instance of a particular adapted amplicon within the transcriptome library:

TABLE 4
Read Counts For First 100 Amplicons Sequenced Using Transcriptome Primer Panel

| Gene | Target | Lib'y # 1 | Lib'y # 2 | Lib'y # 3 | Lib'y # 4 | Lib'y # 5 | Lib'y # 6 |
|---|---|---|---|---|---|---|---|
| SEC24B- | AMPL37741840 | 15 | 19 | 14 | 19 | 13 | 14 |
| AS1 | | | | | | | |
| A1BG | AMPL17425613 | 32 | 1 | 41 | 43 | 1 | 8 |
| A1CF | AMPL36593459 | 115 | 0 | 126 | 116 | 0 | 0 |
| GGACT | AMPL17367653 | 0 | 0 | 4 | 5 | 0 | 5 |
| A2M | AMPL1384 | 2028 | 816 | 2222 | 2024 | 900 | 843 |
| A2ML1 | AMPL35942968 | 0 | 0 | 0 | 0 | 0 | 0 |
| A2MP1 | AMPL37631703 | 0 | 3 | 0 | 0 | 1 | 1 |
| A4GALT | AMPL31888788 | 105 | 51 | 98 | 76 | 51 | 58 |
| A4GNT | AMPL32378916 | 0 | 0 | 0 | 0 | 0 | 0 |
| AAAS | AMPL33679306 | 538 | 122 | 525 | 466 | 139 | 163 |
| AACS | AMPL36995895 | 373 | 525 | 355 | 394 | 541 | 492 |
| AADAC | AMPL5825582 | 2 | 0 | 0 | 2 | 0 | 0 |
| AADACL2 | AMPL22311173 | 0 | 0 | 0 | 0 | 0 | 0 |
| AADACL3 | AMPL4449457 | 0 | 0 | 0 | 0 | 0 | 0 |
| AADACL4 | AMPL612401 | 0 | 0 | 0 | 0 | 0 | 0 |
| AADAT | AMPL32613953 | 177 | 188 | 215 | 168 | 172 | 213 |
| AAGAB | AMPL14432082 | 972 | 793 | 1112 | 1026 | 859 | 794 |
| AAK1 | AMPL25904291 | 17 | 2279 | 15 | 5 | 2428 | 2079 |
| AAMP | AMPL3466806 | 650 | 872 | 807 | 755 | 974 | 998 |
| AANAT | AMPL3275619 | 1 | 0 | 1 | 1 | 0 | 0 |
| AARS | AMPL10784042 | 3746 | 4832 | 4303 | 4085 | 5424 | 4785 |
| AARS2 | AMPL31469233 | 222 | 146 | 171 | 187 | 140 | 156 |
| PTGES3L- | AMPL4663429 | 5 | 9 | 9 | 3 | 12 | 5 |
| AARSD1 | | | | | | | |
| AASDH | AMPL21447136 | 95 | 124 | 108 | 78 | 103 | 123 |
| AASDHPPT | AMPL25092600 | 1130 | 2234 | 1259 | 1123 | 2312 | 2319 |
| AASS | AMPL29326360 | 57 | 73 | 63 | 54 | 61 | 59 |
| AATF | AMPL12558335 | 1877 | 1033 | 2040 | 2136 | 1185 | 1072 |
| AATK | AMPL5542914 | 40 | 1420 | 42 | 51 | 1619 | 1322 |
| ABAT | AMPL33853761 | 328 | 3877 | 371 | 388 | 4871 | 5283 |
| ABCA1 | AMPL28385508 | 331 | 153 | 451 | 410 | 162 | 169 |
| ABCA10 | AMPL18549579 | 3 | 229 | 4 | 1 | 216 | 202 |
| ABCA12 | AMPL15858282 | 49 | 7 | 53 | 65 | 1 | 1 |
| ABCA13 | AMPL34481782 | 1 | 0 | 1 | 0 | 0 | 0 |
| ABCA17P | AMPL19877459 | 1 | 37 | 3 | 0 | 51 | 35 |
| ABCA2 | AMPL8099046 | 771 | 4990 | 755 | 747 | 5947 | 5336 |
| ABCA3 | AMPL5076273 | 337 | 2064 | 384 | 327 | 2542 | 2270 |
| ABCA4 | AMPL3661170 | 11 | 9 | 20 | 15 | 9 | 12 |
| ABCA5 | AMPL16435662 | 376 | 1328 | 374 | 331 | 1576 | 1495 |
| ABCA6 | AMPL18550501 | 1 | 182 | 2 | 5 | 183 | 200 |
| ABCA7 | AMPL31288152 | 56 | 17 | 44 | 46 | 25 | 16 |
| ABCA8 | AMPL13251723 | 32 | 380 | 25 | 26 | 423 | 407 |
| ABCA9 | AMPL15770940 | 0 | 70 | 1 | 0 | 97 | 75 |
| ABCB1 | AMPL5599607 | 71 | 260 | 72 | 66 | 331 | 272 |
| ABCB10 | AMPL12686552 | 519 | 350 | 537 | 483 | 318 | 343 |
| ABCB11 | AMPL7530015 | 1 | 0 | 2 | 0 | 3 | 1 |
| ABCB4 | AMPL5513474 | 29 | 2 | 24 | 15 | 3 | 3 |
| ABCB5 | AMPL6715085 | 4 | 0 | 6 | 7 | 0 | 0 |
| ABCB6 | AMPL29447135 | 539 | 301 | 532 | 513 | 313 | 289 |
| ABCB7 | AMPL28554255 | 1 | 0 | 3 | 0 | 1 | 0 |
| ABCB8 | AMPL12342162 | 51 | 103 | 91 | 63 | 91 | 94 |
| ABCB9 | AMPL30785555 | 5 | 10 | 4 | 0 | 14 | 20 |
| ABCC1 | AMPL28603395 | 476 | 63 | 562 | 414 | 109 | 69 |
| ABCC10 | AMPL7136100 | 113 | 38 | 187 | 109 | 59 | 44 |
| ABCC11 | AMPL16427617 | 2 | 8 | 0 | 1 | 3 | 6 |
| ABCC12 | AMPL16211125 | 1 | 0 | 0 | 0 | 0 | 0 |
| ABCC2 | AMPL3093849 | 523 | 9 | 644 | 601 | 8 | 6 |
| ABCC3 | AMPL11367250 | 77 | 4 | 38 | 61 | 0 | 2 |
| ABCC4 | AMPL27233725 | 300 | 30 | 287 | 297 | 49 | 54 |
| ABCC5 | AMPL28800826 | 439 | 866 | 521 | 503 | 1030 | 964 |
| ABCC6 | AMPL9526889 | 16 | 0 | 18 | 15 | 0 | 2 |
| ABCC8 | AMPL1955595 | 1 | 307 | 6 | 2 | 346 | 250 |
| ABCC9 | AMPL31159527 | 1 | 27 | 1 | 1 | 31 | 38 |
| ABCD1 | AMPL4354534 | 396 | 62 | 461 | 431 | 65 | 64 |
| ABCD2 | AMPL30300100 | 10 | 523 | 10 | 15 | 572 | 576 |
| ABCD3 | AMPL10799498 | 565 | 824 | 582 | 574 | 903 | 802 |
| ABCD4 | AMPL28234371 | 271 | 354 | 293 | 293 | 354 | 278 |
| ABCE1 | AMPL8835806 | 2894 | 1317 | 3492 | 3232 | 1534 | 1501 |
| ABCF1 | AMPL1062180 | 1733 | 632 | 1978 | 1863 | 677 | 649 |
| ABCF2 | AMPL28710948 | 1784 | 1181 | 1885 | 1763 | 1390 | 1238 |
| ABCF3 | AMPL33247607 | 305 | 398 | 389 | 286 | 580 | 400 |
| ABCG1 | AMPL29470197 | 40 | 63 | 44 | 36 | 50 | 40 |
| ABCG2 | AMPL28876904 | 153 | 613 | 159 | 178 | 644 | 628 |
| ABCG4 | AMPL35962683 | 11 | 267 | 4 | 7 | 360 | 313 |
| ABCG5 | AMPL37320861 | 50 | 7 | 57 | 55 | 7 | 7 |
| ABCG8 | AMPL37439385 | 4 | 3 | 9 | 7 | 8 | 3 |
| ABHD1 | AMPL16990403 | 34 | 22 | 35 | 41 | 36 | 35 |
| ABHD10 | AMPL34005486 | 189 | 256 | 197 | 147 | 286 | 219 |
| ABHD11 | AMPL35722665 | 140 | 95 | 141 | 135 | 96 | 69 |
| ABHD12 | AMPL32297518 | 1243 | 1842 | 1434 | 1277 | 1999 | 1744 |
| ABHD12B | AMPL7873511 | 0 | 19 | 0 | 2 | 9 | 16 |
| ABHD13 | AMPL16027178 | 50 | 90 | 67 | 35 | 96 | 109 |
| ABHD14A | AMPL25862037 | 81 | 448 | 75 | 79 | 446 | 423 |
| ABHD14B | AMPL6719779 | 641 | 118 | 632 | 529 | 115 | 139 |
| ABHD15 | AMPL20585213 | 180 | 46 | 176 | 142 | 55 | 61 |
| ABHD16A | AMPL36420922 | 319 | 523 | 358 | 322 | 592 | 639 |
| ABHD16B | AMPL17532477 | 66 | 28 | 86 | 47 | 20 | 24 |
| ABHD2 | AMPL17078991 | 1491 | 2980 | 1603 | 1483 | 3212 | 2888 |
| ABHD3 | AMPL17297898 | 705 | 243 | 740 | 717 | 284 | 264 |
| ABHD4 | AMPL37127761 | 290 | 213 | 324 | 320 | 229 | 206 |
| ABHD5 | AMPL33305114 | 458 | 365 | 482 | 426 | 401 | 405 |
| ABHD6 | AMPL32219778 | 260 | 986 | 261 | 265 | 1215 | 1212 |
| ABHD8 | AMPL37207261 | 107 | 700 | 140 | 132 | 734 | 696 |
| ABI1 | AMPL983412 | 1059 | 1570 | 1107 | 975 | 1672 | 1625 |
| ABI2 | AMPL27003455 | 515 | 1134 | 517 | 460 | 1417 | 1245 |
| ABI3 | AMPL32495296 | 42 | 161 | 39 | 25 | 180 | 160 |
| ABI3BP | AMPL25539060 | 144 | 118 | 143 | 152 | 131 | 135 |
| ABL1 | AMPL28966626 | 2127 | 732 | 2199 | 2190 | 966 | 905 |
| ABL2 | AMPL27388698 | 1038 | 1099 | 1276 | 1138 | 1394 | 1201 |
| ABLIM1 | AMPL8548910 | 0 | 2 | 0 | 0 | 1 | 0 |

What is claimed:

1. A method for detecting a plurality of polynucleotides in a sample, comprising:
a) contacting, within a single reaction mixture, a plurality of target-specific primer pairs with a plurality of target polynucleotides derived from the sample, under nucleic acid hybridization conditions such that different target-specific primer pairs hybridize to different target polynucleotides, wherein the different target-specific primer pairs include at least one cleavable group;
b) extending the target-specific primer pairs to form a plurality of amplicons which contain a sequence derived from a target polynucleotide and a primer-derived sequence which includes the at least one cleavable group;
c) cleaving the at least one cleavable group of the plurality of amplicons to generate a plurality of cleaved amplicons; and
d) joining one or both ends of the plurality of cleaved amplicons to a Y-shaped adaptor to form a plurality of adaptor-joined amplicons.

2. The method of claim 1, further comprising: (e) detecting the adaptor-joined amplicons.

3. The method of claim 2, wherein the detecting comprises sequencing the plurality of adaptor-joined amplicons.

4. The method of claim 1, further comprising: (e) re-amplifying the plurality of adaptor-joined amplicons.

5. The method of claim 1, further comprising: (e) attaching the adaptor-joined amplicons to a support or to a plurality of supports.

6. The method of claim 5, wherein the support includes a plurality of first and second capture primers attached thereon, and the plurality of first and second capture primers have different sequences.

7. The method of claim 6, wherein the adaptor-joined amplicons are attached to the support by:
a) rendering the adaptor-joined amplicons single-stranded to generate single-stranded adaptor-joined molecules;
b) hybridizing the single-stranded adaptor-joined molecules to the plurality of first capture primers; and
c) extending the plurality of first capture primers to generate a plurality of first capture primer extension products.

8. The method of claim 5, wherein the plurality of supports includes a plurality beads with a plurality of capture primers attached thereon.

9. The method of claim 8, wherein the adaptor-joined amplicons are attached to the support by: conducting a recombinase polymerase amplification (RPA) reaction under an isothermal amplification condition with: (i) a polymerase; (ii) a plurality of nucleotides; (iii) a recombinase; (iv) a recombinase loading factor; (v) a single-stranded binding protein; and (vi) a plurality of soluble reverse primers.

10. The method of claim 3, wherein the sequencing is conducted by polymerase-mediated incorporation of a terminator nucleotide which is blocked at the 2′ or 3′ OH sugar position of the base.

11. The method of claim 10, wherein the terminator nucleotide is attached with an optically-detectable dye.

12. The method of claim 3, wherein the sequencing is conducted by polymerase-mediated incorporation of a non-labeled and non-blocked nucleotide.

13. The method of claim 3, wherein the sequencing is conducted by detecting changes in release of protons, hydrogen ions, charge transfer or heat.

14. The method of claim 1, comprising:
a) contacting, within a single reaction mixture, a plurality of target-specific primer pairs with a plurality of target polynucleotides derived from the sample, wherein the plurality of target polynucleotides includes at least a first and a second target polynucleotide, under nucleic acid hybridization conditions such that different target-specific primer pairs hybridize to different target polynucleotides, wherein the different target-specific primer pairs include at least one cleavable group;
b) extending the target-specific primer pairs to form a plurality of first amplicons which contain a sequence derived from the first target polynucleotide and a primer-derived sequence which includes the at least one cleavable group, and extending the target-specific primer pairs to form a plurality of second amplicons which contain a sequence derived from the second target polynucleotide and a primer-derived sequence which includes the at least one cleavable group;
c) cleaving the at least one cleavable group of the first and second plurality of amplicons to generate a plurality of first and second cleaved amplicons;
d) joining one or both ends of the plurality of first and second cleaved amplicons to a Y-shaped adaptor to form a plurality of first and second adaptor-joined amplicons; and
e) detecting the first and second adaptor-joined amplicons.

15. The method of claim 14, wherein the detecting the first and second adaptor-joined amplicons comprises: determining an amount of amplicons containing a sequence derived from the first target polynucleotide and determining an amount of amplicons containing a sequence derived from the second target polynucleotide.

16. The method of claim 14, wherein the detecting the first and second adaptor-joined amplicons comprises: quantifying the amount of the first target polynucleotide in the sample, and quantifying the amount of the second target polynucleotide present in the sample.

17. The method of claim 14, further comprising calculating a ratio of the amount of adaptor-joined amplicons derived from the first target polynucleotide, and the amount of adaptor-joined amplicons derived from the second target polynucleotide.

18. The method of claim 14, wherein the detecting comprises sequencing the plurality of adaptor-joined amplicons.

19. The method of claim 1, wherein the Y-shaped adaptor is joined to one or both ends of the plurality of cleaved amplicons by enzymatic ligation.

20. The method of claim 1, wherein the Y-shaped adaptor includes a unique identifier sequence or a degenerate sequence.

21. The method of claim 1, wherein the Y-shaped adaptor includes a sequencing primer binding site, an amplification primer binding site or a restriction enzyme recognition sequence.

22. The method of claim 1, wherein the Y-shaped adaptor includes a 5′ or 3′ overhang end.

23. The method of claim 1, wherein the different target-specific primer pairs are tailed primer pairs or non-tailed primer pairs.

24. The method of claim 1, wherein the cleavable group is cleavable with an enzyme, chemical compound, heat or light.

25. The method of claim 1, wherein the cleavable group is cleavable with a uracil DNA glycosylase (UDG) or a formamidopyrimidine DNA glycosylase (Fpg).

26. The method of claim 1, wherein the cleavable group is cleavable with a FuPa reagent which includes a DNA polymerase, a uracil-cleaving enzyme, and an antibody that inhibits activity of the DNA polymerase.

27. The method of claim 1, wherein the at least one cleavable group comprises uracil, uridine, inosine, or 7,8-dihydro-8-oxoguanine (8-oxoG) nucleobases.

28. The method of claim 1, wherein the plurality of target-specific primer pairs includes 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target-specific primer pairs.

29. The method of claim 1, wherein the extending in step (b) includes forming a plurality of amplicons containing sequences derived from 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target polynucleotides.

30. The method of claim 2, wherein detecting the adaptor-joined amplicons comprises: quantifying the amount of adaptor-joined amplicons containing sequence derived from the 2-100, or 100-500, or 500-1,000, or 1,000-5,000, or 5,000-10,000, or 10,000-15,000, or 15,000-20,000, or 20,000-25,000, or 25,000-50,000 or 50,000-100,000 different target polynucleotides.

31. The method of claim 1, wherein the sample includes DNA, RNA or cDNA.

32. The method of claim 1, wherein the sample include cell-free DNA or cell-free RNA.

33. The method of claim 1, wherein the sample is derived from a single cell or a population of cells.

34. The method of claim 1, wherein the sample is derived from prokaryotes, eukaryotes, fungus or virus.

35. The method of claim 1, wherein the sample is derived from a bodily fluid selected from a group consisting of blood, urine, serum, lymph, tumor, saliva, anal and vaginal secretions, amniotic samples, perspiration, and semen.
